(19) United States
(12) Patent Application Publication
(10) Pub. No.: US 2011/0045027 A1
(43) Pub. Date: Feb. 24, 2011

(54) Adjuvant

(75) Inventors: Ian Richard Catchpole, Hertfordshire (GB); Irene Papanicolaou, Hertfordshire (GB)

Correspondence Address: GLAXOSMITHKLINE;GLOBAL PATENTS, FIVE MOORE DR., PO BOX 13398, MAIL STOP: C.2111F, RESEARCH TRIANGLE PARK, NC, 27709-3398, US

(73) Assignee: Glaxo Group Limited, Middlesex (GB)

(21) Appl. No.: 11/917,961

(22) PCT Filed: Jun. 20, 2006
(86) PCT No.: PCT/EP2006/006264

§ 371 Date: Jul. 13, 2010

(30) Foreign Application Priority Data
Jun. 22, 2005 (GB) 0512751.9

(51) Int. Cl. A61K 39/00; A61P 37/04

(52) U.S. Cl. 424/278.1

(57) ABSTRACT

The present invention provides a novel adjuvant for polynucleotide vaccines, and in particular the present invention provides immunogenic compositions comprising a polynucleotide encoding an antigen capable of eliciting an immune response and an adjuvant comprising an immunostimulatory quantity of a gemini surfactant, or a derivative thereof.

The present invention provides a novel adjuvant for polynucleotide vaccines, and in particular the present invention provides immunogenic compositions comprising a polynucleotide encoding an antigen capable of eliciting an immune response and an adjuvant comprising an immunostimulatory quantity of a gemini surfactant, or a derivative thereof.

The invention further comprises polynucleotide vaccines that comprise, or are administered in association with, a composition that comprises a gemini surfactant compound. The polynucleotide vaccines of the present invention are vaccines that encode an antigen against which it is desired to generate an immune response, and in particular the polynucleotide vaccine may be a DNA vaccine. Also provided by the present invention is the use of gemini surfactants in the manufacture of a polynucleotide vaccine composition for the purpose of enhancing the immune response against the specific antigen that is encoded by the polynucleotide vaccine. Vaccine compositions, kits comprising separate polynucleotide composition and adjuvant compositions for separate or simultaneous administration, methods of manufacture of the vaccines and kits, and methods of treatment of individuals with the immunogenic compositions and vaccines of the present invention, are provided.

### BACKGROUND

Vaccines have for many years included substances that have a direct or indirect stimulatory effect on the immune system, termed “adjuvants”, such that the magnitude or quality of the immune response is altered or augmented. General information about the use of adjuvants is provided in Powell, M. F. & Newman, M. J. (eds.) (1995) Vaccine Design—The Subunit and Adjuvant Approach. Plenum Press, New York and London.

Adjuvants for protein or polysaccharide vaccines, or in general those vaccines which comprise the antigen itself, have been known and developed since the 1920s. In contrast, polynucleotide vaccines where the vaccine comprises a polynucleotide that encodes the antigen and facilitates production in the host cells of the vaccinee, are themselves a relatively recent development. Necessarily therefore, less is known about polynucleotide vaccine adjuvants. The adjuvant strategy for polynucleotide vaccines often involves the co-expression of immune modifiers, such as cytokines, together with the antigen. Recently described polynucleotide vaccine adjuvants include small molecules such as tucerasol (WO 00/12121), imidazoquinoline amines (WO 02/24225, WO 03/077944) and inducible nitric oxide synthase (iNOS) inhibitors (WO 03/030935).

Surfactants are substances that markedly affect the surface properties of a liquid, even at low concentrations. For example surfactants will significantly reduce surface tension when dissolved in water or aqueous solutions and will reduce interfacial tension between two liquids or between a liquid and a solid. This property of surfactant molecules has been widely exploited in industry, particularly in the detergent and oil industries. In the 1970s a new class of surfactant molecule was reported, characterised by two hydrophobic chains with polar heads which are linked by a hydrophobic bridge (Deinega, Y et al., Kolloidn. Zh. 36, 649, 1974). These molecules, which have been termed “gemini” (Menger, F M and Littau, C A, J. Am. Chem. Soc. 113, 1451, 1991), have very desirable properties over their monomeric equivalents. For example they are highly effective in reducing interfacial tension between oil and water based liquids and have a very low critical micelle concentration (Menger, F M and Keiper, J S, Angewandte. Chem. Int. Ed. Engl., 2000, 39, 1906).

Cationic surfactants have been used inter alia for the transfection of polynucleotides into cells in culture, and there are examples of such agents available commercially to scientists involved in genetic technologies (for example the reagent Tfx™-50 for the transfection of eukaryotic cells available from Promega Corp. WI, USA).

The efficient delivery of DNA to cells in vivo, either for gene therapy or for antisense therapy, has been a major goal for some years. Much attention has concentrated on the use of viruses as delivery vehicles, for example adenoviruses for epithelial cells in the respiratory tract with a view to corrective gene therapy for cystic fibrosis (CF). However, despite some evidence of successful gene transfer in CF patients, the adenovirus route remains problematic due to inflammatory side-effects and limited transient expression of the transferred gene. Several alternative methods for in vivo gene delivery have been investigated, including studies using cationic surfactants. Gao, X et al. Gene Ther. 2, 710-722, 1995 demonstrated the feasibility of this approach with a normal human gene for CF transmembrane conductance regulator (CFTR) into the respiratory epithelium of CF mice using amine carrying cationic lipids. This group followed up with a liposomal CF gene therapy trial which, although only partially successful, demonstrated the potential for this approach in humans (Caplen, N J. et al., Nature Medicine, 1, 39-46, 1995). More recently other groups have investigated the potential of other cationic lipids for gene delivery (Miller, A, Angew. Int. Ed. Engl., 37, 1768-1785, 1998), for example cholesterol derivatives (Oudrhiri, N et al. Proc. Natl. Acad. Sci. 94, 1651-1656, 1997). This limited study demonstrated the ability of these cholesterol based compounds to facilitate the transfer of genes into epithelial cells both in vitro and in vivo, thereby lending support to the validity of this general approach.

These studies, and others, have led the way for the development of low-toxicity surfactant molecules which facilitate the effective transfer of polynucleotides into cells both in vitro for transfection in cell-based experimentation and in vivo for gene therapy and antisense treatments. Gemini surfactants based on cysteine (WO99/29712) or on spermine (WO00/77032) or diamine (WO00/76954) have previously been made. Other examples of gemini surfactants are found in WO00/27795, WO02/30957, WO02/50100 and WO03/82809. The use of gemini surfactants as polynucleotide vectors has recently been reviewed (A. J. Kirby, P. Camilleri, J. B. F. N. Engberts, M. C. Feiters, R. J. M. Nolte, O, Söderman, M. Bergsma, P. C. Bell, M. L. Fielden, C. L. García Rodríguez, Philippe Guédat, A. Kremer, C. McGregor, C. Perrin, G. Ronsin and M. C. P. van Eijk, Angew. Chem. Int. Ed., 2003, 42, 1448, see also R. Zana and J. Xia, Gemini Surfactants, Marcel Dekker, NY, 2004).

### SUMMARY OF THE INVENTION

The present invention provides an immunogenic composition comprising a polynucleotide encoding an antigen capable of eliciting an immune response and an adjuvant comprising an immunostimulatory quantity of a gemini surfactant, or a derivative thereof.

In one embodiment of the invention, the immunogenic compositions of the present invention are in the form of polynucleotide vaccines comprising (a) a polynucleotide vaccine component that encodes an antigen against which it is desired to generate an immune response, and (b) an adjuvant composition comprising an immune stimulatory (immunostimulatory) quantity of a gemini surfactant, or a derivative thereof.

In the context of the present invention the polynucleotide vaccine encoding the vaccine antigen is any polynucleotide or vector that is capable of directing expression of the said antigen in the cells of the host vaccinee. The vector may be a live or attenuated viral or bacterial vector which delivers the foreign sequence that encodes the vaccine antigen.

In one aspect of the present invention the vaccines comprise a polynucleotide vector which is a DNA plasmid vector. In this aspect of the present invention the plasmid vector may be delivered to the vaccinee in liquid form, or in the form of dense micro-beads suitable for ballistic delivery into the skin, or formulated on the surface of dense micro-beads suitable for ballistic delivery into the skin, or coated onto microneedles.

In other embodiments of the present invention the vaccines of the present invention may be in solid form, such that the polynucleotide may be in a “dry” form and co-formulated with the gemini. For example, the polynucleotide antigen and the gemini may be in dry solid solution within a solid, or glassy, matrix. In such an embodiment the solid matrix may be a carbohydrate, or sugar, in solid form.

In one form of the present invention the polynucleotide and gemini compound are provided on the surface of microbeads suitable for ballistic delivery into the epidermis.

In a related aspect of the present invention the solid vaccine formulation may comprise a protein antigen and a gemini compound. Further provided in this related aspect of the present invention is a method of stabilising a protein in its dry state, such as in its lyophilised form, by co-formulating said protein with the gemini compound. This formulation and method has the additional advantage of enhancing the immune responses raised by the antigen.

The dose of the gemini compound in the vaccines of the present invention is sufficient to enhance the immune response against the antigen.

The vaccines of the present invention are particularly adapted, by the formulation with the adjuvants described herein, to the provision of highly potent immune responses, including cell mediated immune responses. In addition, the vaccines of the present invention are also highly stable compositions, in that the stability of the polynucleotides in the vaccine is enhanced by the presence of gemini surfactant. An additional advantage of the present invention is the provision of a vaccine/adjuvant composition that does not have the toxicity issues associated with the persistence of potentially toxic adjuvants in the body of the vaccine.

In a further aspect of the present invention the immunogenic composition may contain one or more additional adjuvant, for example an immunostimulatory cytokine such as IL-2, GM-CSF or IFN-γ, or for example Imiquimod, or for example a Toll like receptor (TLR) 4 ligand, in combination with a saponin, or for example CpG.

### DETAILED DESCRIPTION OF THE INVENTION

The present invention provides an immunogenic composition comprising a polynucleotide encoding an antigen capable of eliciting an immune response and an adjuvant comprising an immunostimulatory quantity of a gemini surfactant, or a derivative thereof

In one embodiment of the invention, the immunogenic compositions of the present invention are in the form of polynucleotide vaccines comprising (a) a polynucleotide vaccine component that encodes an antigen against which it is desired to generate an immune response, and (b) an adjuvant composition comprising an immunostimulatory quantity of a gemini surfactant or a derivative thereof.

The polynucleotide elements forming part of the immunogenic compositions and/or vaccines of the present invention are vectors which, when administered to a vaccine in an appropriate form, drive expression of an antigen in the cells of the vaccine, thereby generating an immune response against the antigen.

The vectors or polynucleotide elements of the immunogenic compositions and/or vaccines of the present invention, which encode the antigen against which it is desired to generate an immune response, are operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes, live or attenuated bacterial, viral or phage vectors.

Examples of suitable viral vectors include herpes simplex viral vectors, vaccinia or alpha-virus vectors and retroviruses, including lentiviruses, human and simian adenoviruses and adeno-associated viruses.

In an important aspect of the present invention the polynucleotide is in the form of a DNA plasmid comprising an expression cassette having a promoter region and a coding region.

Any suitable promoter may be used in the polynucleotides of the present invention. Promoters and other expression regulation signals that form part of the polynucleotide vectors may be selected to be compatible with the host cell for which expression is designed.

For example, mammalian promoters include the metallothionein promoter, which can be induced in response to heavy metals such as cadmium, and the β-actin promoter. Viral promoters such as the SV40 large T antigen promoter; human cytomegalovirus (CMV) immediate early (IE) promoter, for example wherein the 5′ untranslated region of the HCMV IE gene comprising exon 1 is included as described in WO 02/36792; rous sarcoma virus LTR promoter; adenovirus promoter; or a HPV promoter, particularly the HPV upstream regulatory region (URR) may also be used. All these promoters are well described and readily available in the art.

The coding region encodes an antigen which, once expressed in the host cells of the vaccinee, generates an immune response. In one embodiment of the present invention, the polynucleotide encodes one or more of Nef, Gag, RT, Pol, Env, P501, Mage-3, Her-2/Neu or immunogenic derivatives or fragments thereof, for example in one embodiment the polynucleotide encodes one or more HIV antigens, for example Gag, Nef, Pol or Env, or immunogenic derivatives or fragments thereof.

Optionally the coding region, or an additional coding region, may encode an additional adjuvant for example, an immunostimulatory cytokine such as IL-2, GM-CSF or IFN-γ.

In one aspect of the present invention, there is provided a vaccine composition comprising a gemini surfactant or a salt, solvate, or physiologically functional derivative thereof, and a polynucleotide which encodes an antigen against which it is desired to generate an immune response.

As used herein, the term “gemini”, “gemini compound” or “gemini surfactant” refers to a compound having the following characteristics:
a) a hydrophilic head group comprising one or two groups each comprising one or more amino acids or amines; linked to
b) two or three hydrophobic hydrocarbyl chains of up to 24 carbons.
   Examples of such gemini compounds are compounds having two hydrocarbyl chains linked to either:
   a) a peptide-based hydrophilic head group (for example as disclosed in WO99/29712);
   b) a carbohydrate-based hydrophilic head group (for example as disclosed in WO00/76854);
   c) a spermine-based hydrophilic head group (for example as disclosed in WO00/77032);
   d) a diamine-based hydrophilic head group (for example as disclosed in WO00/76954);
   e) a hydrophilic head group based on diaminodicarboxylic acid derivatives (for example as disclosed in WO02/50100;
   f) a diaminoacid polyamine hydrophilic head group (for example as disclosed in WO03/82809).

Further examples of such gemini compounds include those comprising two hydrocarbyl chains linked to a spermidine-based hydrophilic head group; those comprising two or three hydrocarbyl chains linked to a pentamine-based hydrophilic head group, and those comprising two hydrocarbyl chains linked by ester linkage to a hydrophilic headgroup, examples of which are described hereinbelow.

In one embodiment of the present invention the adjuvant is a gemini surfactant selected from those comprising two hydrocarbyl chains linked to a spermine-based hydrophilic head group; comprising two hydrocarbyl chains linked to a spermidine-based hydrophilic head group; those comprising two or three hydrocarbyl chains linked to a pentamine-based hydrophilic head group, and those comprising two hydrocarbyl chains linked by ester linkage to a hydrophilic headgroup, for example wherein the gemini surfactant comprises two hydrocarbyl chains linked to a hydrophilic headgroup, for example wherein the hydrocarbyl chains are linked to a hydrophilic headgroup by an ester linkage. In one embodiment of the present invention the gemini surfactant is selected from GS092A and GS543A.

In one aspect the present invention provides a vaccine comprising the immunogenic composition of the invention. Such a vaccine may be useful in a therapeutic or prophylactic setting.

The present invention further provides a method of eliciting an immune response in a mammalian subject comprising administering to said individual an immunogenic composition of the present invention, for example a method of eliciting an immune response in a human subject. In one aspect of the present invention the method is for eliciting a therapeutically effective immune response. In another aspect the method is for eliciting a protective immune response.

In one embodiment the invention provides a method of treating an individual infected with HIV, HPV, HCV or cancer, comprising administration to that individual of an immunogenic composition according to the invention.

In one embodiment the present invention provides the use of an immunogenic composition according to the present invention in the manufacture of a medicament for the amelioration or treatment of HIV, HPV, HCV or cancer.

In a further embodiment of the present invention, one or more adjuvants or polynucleotides encoding an adjuvant may be co-administered with the immunogenic composition of the invention. Examples of suitable adjuvants include GM-CSF and TLR agonists such as imiquimod. Imiquimod is commercially available as Aldara™ cream (3M). These adjuvants and the combination of these two adjuvant components are described in WO2005025614. Other suitable adjuvants may comprise a Toll like receptor (TLR) 4 ligand, in combination with a saponin. The Toll like receptor (TLR) 4 ligand may be for example, an agonist such as a lipid A derivative particularly monophosphoryl lipid A or more particularly 3 Deacylated monophosphoryl lipid A (3 D-MPL). 3 D-MPL is sold under the trademark MPL® by Corixa Corporation and primarily promotes CD4+ T cell responses with an IFN-g (Th1) phenotype. It can be produced according to the methods disclosed in GB 2220211A. Chemically, it is a mixture of 3-deacylated monophosphoryl lipid A with 3, 4, 5 or 6 acylated chains. In one embodiment of the invention, small particle 3 D-MPL may be used. Small particle 3 D-MPL has a particle size such that it may be sterile-filtered through a 0.22 μm filter. Such preparations are described in PCT Patent Application WO 9421292.

The adjuvant may also comprise one or more synthetic derivatives of lipid A which are known to be TLR 4 agonists including, but not limited to:

OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate) as described in PCT Patent Application WO 95/14026.

OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) as described in WO 9964301 and WO 00/0462.

OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used include, but are not limited to, alkyl Glucosaminide phosphates (AGPs) such as those disclosed in WO 9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both can be used as one or more adjuvants in the compositions of the invention. Alternatively such immunogenic compositions further comprise a CpG oligonucleotide alone or together with an aluminium salt.

Another example of an immunostimulant is an immunostimulatory oligonucleotide containing unmethylated CpG dinucleotides (“CpG”). CpG is an abbreviation for cytosine-guanosine dinucleotide motifs present in DNA. CpG is known in the art as being an adjuvant when administered by both systemic and mucosal routes (WO 96/02555, EP 468520, Davis et al., J. Immunol, 1998, 160(2):870-876; McCluskie and Davis, J. Immunol., 1998, 161(9):4463-6). Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p 546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequences containing oligonucleotides can activate various immune subsets, including natural killer cells (which produce interferon and have cytolytic activity) and macrophages (Wooldrige et al Vol 89 (no. 8), 1977). Other unmethylated CpG containing sequences not having this consensus sequence have also now been shown to be immunomodulatory.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8).

Such immunostimulants as described above may be formulated together with carriers, such as for example liposomes, oil in water emulsions, and or metallic salts, including aluminium salts (such as aluminium hydroxide). For example, 3D-MPL may be formulated with aluminium hydroxide (EP 0 689 454) or oil in water emulsions (WO 95/17210); QS21 may be advantageously formulated with cholesterol containing liposomes (WO 96/33739), oil in water emulsions (WO 95/17210) or alum (WO 98/15287); CpG may be formulated with alum (Davis et al. supra; Brazolot-Millan supra) or with other cationic carriers.

As used herein the term “physiologically functional derivative” refers to any pharmaceutically acceptable derivative of an adjuvant of the present invention (formed, for example, by addition of alkyl, alkenyl, alkynyl, aryl or polysaccharide groups to oxidised nitrogen atoms of the gemini compound), which upon administration to a mammal is itself capable of enhancing the immune response against the antigen encoded by the polynucleotide, or is capable of indirectly doing so through the action of a breakdown product formed from the derivative in situ after administration to the body. Such derivatives are clear to those skilled in the art, without undue experimentation, and with reference to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “solvate” refers to a complex of variable stoichiometry formed by a solute (in this invention a gemini compound or a salt or physiologically functional derivative thereof) and a solvent. Such solvents for the purpose of the invention may not interfere with the biological activity of the solute.

Examples of suitable solvents include, but are not limited to, water, methanol, ethanol and acetic acid. In one embodiment the solvate is boric acid.

Typically, the salts of the present invention are pharmaceutically acceptable salts. Salts encompassed within the term “pharmaceutically acceptable salts” refer to non-toxic salts of the compounds of this invention. Salts of the compounds of the present invention may comprise salts derived from a nitrogen on a substituent in the compound of formula (I). Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, tris(hydroxymethyl)aminomethane and valerate.

Suitable salts of the gemini compound, or derivatives thereof, include most metals including sodium, potassium, lithium, calcium, magnesium, zinc. Ammonium salts are also known and a guanidinium salt. These salts may be solvated with water.

In one embodiment of the present invention the gemini surfactant is a symmetrical gemini. Symmetrical geminis are those compounds where, if two amino acid or amine containing groups are present in the hydrophilic head group, both groups are the same as each other and all the hydrophobic chains are the same as each other. In other embodiments of the present invention the gemini surfactant is present in an unsymmetrical form. Unsymmetrical geminis are those compounds where, if two amino acid or amine containing groups are present in the hydrophilic head group, the two groups are different from each other, for example where one group is based on one amino acid and the other group is based on another amino acid; or, alternatively, where the hydrophobic hydrocarbyl chains are different, for example where one hydrocarbyl chain is derived from oleic acid and the other is derived from stearic acid. Unsymmetrical geminis also encompass those compounds where both amino acid/amine groups are different and also both hydrophobic chains are different. The gemini surfactant may be present as a salt, solvate or other physiologically active derivative thereof.

In another embodiment of the present invention, the vaccine adjuvant component comprises a combination of two or more gemini surfactants. Accordingly, when two components are present, the adjuvant compositions may comprise a combination of two symmetrical or two unsymmetrical or one symmetrical and one unsymmetrical Gemini compounds.

In one embodiment the gemini compound is a spermine-based compound of formula (I):

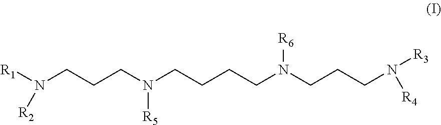

where R₁ and R₃ are hydrogen and R₂ and R₄, which may be the same or different, are hydrogen or peptide groups formed from one or more amino acids linked together, in a linear or branched manner, by amide (CONH) bonds and further linked to the spermine backbone by amide bonds, having the general formula (II):

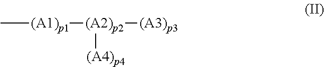

where p1 is 0 to 5 and p2 is 1 to 5, for example 1; and the values for p3 and p4, which may be the same or different, are from 0 to 5, for example 0;
A1, A3 and A4, which may be the same or different, are amino acids selected from serine, lysine, ornithine, threonine, histidine, cysteine, arginine, tyrosine, diaminobutyric acid (dab) and diaminopropionic acid (dap); and
A2 is an amino acid selected from lysine, ornithine and histidine;
and R₅ and R₆, which may be the same or different, are saturated or unsaturated hydrocarbyl groups having up to 24 carbon atoms and linked to the spermine backbone by an amide or an amine (NCH₂) linkage;
or
where R₁ and R₃ are hydrogen, R₂ and R₄, which may be the same or different are saturated or unsaturated hydrocarbyl groups having up to 24 carbon atoms and linked to the spermine backbone by amide or amine bonds, and R₅ and R₆, which may be the same or different, are peptide groups of formula (II) linked to the spermine backbone by amide bonds;
or
a salt, for example a pharmaceutically acceptable salt thereof.

In one embodiment of the invention R₅ and R₆ are saturated or unsaturated hydrocarbyl groups having up to 24 carbon atoms and linked to the spermine backbone by an amide or an amine (NCH₂) linkage; R₁ and R₃ are hydrogen and R₂ and R₄ are both lysine.

In a further embodiment R₅ and R₆ are saturated or unsaturated hydrocarbyl groups having up to 24 carbon atoms and linked to the spermine backbone by an amide or an amine (NCH₂) linkage; R₁, R₃ and R₂ are hydrogen and R₄ is lysine (p1 is 1 and p2, p3 and p4 are all 0; A1 is lysine).

In yet a further embodiment R₅ and R₆ are saturated or unsaturated hydrocarbyl groups having up to 24 carbon atoms and linked to the spermine backbone by an amide or an amine (NCH₂) linkage; R₁, R₃ and R₂ are hydrogen and R₄ is serine-lysine (p1 and p2 are both 1 and p3 and p4 are both 0; A1 is serine and A2 is lysine).

Methods for making spermine geminis for use in the present invention are disclosed in WO00/77032.

A general scheme for making unsymmetrical spermine-based geminis is shown in FIG. 26.

In another embodiment the gemini compound is a pentamine-based compound of formula (III):

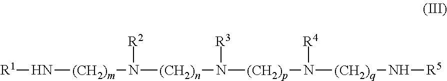

wherein
m is 1 to 6;
q is 1 to 6;
n is 1 to 10;
p is 1 to 10;
R¹, R², R³, R⁴ and R⁵, which may be the same or different, is each selected from hydrogen, R^{w}, or (Aa)ₓ;
where R^{w} is a saturated or unsaturated, branched or unbranched aliphatic carboxylic acid of up to 24 carbon atoms linked as its amide derivative, and wherein at least two R^{w} groups are present in the molecule;
(Aa)ₓ, which may be the same or different at each occurrence, is a series of x natural or unnatural amino acids linked in a linear or branched manner;
x is 0 to 6.
or a salt, for example a pharmaceutically acceptable salt thereof.

In one embodiment m is 2 or 3, for example 3.

In another embodiment q is 2 or 3, for example 3.

In a further embodiment n is 3 to 6, for example 4.

In a still further embodiment p is 3 to 6, for example 3.

(Aa) is, for example a basic amino acid. Examples of basic amino acids include [H₂N(CH₂)₃]₂N(CH₂)CO₂H, (H₂NCH₂)₂CHCO₂H, or L or D enantiomers of Ser, Lys, Orn, Dab (Diamino butyric acid) or Dap (diamino propionic acid). For example, the amino acid (Aa) may be an amino acid comprising an amino group (or optionally an OH group) in its side chain and comprising not more that 12 carbon atoms in total, for example not more that 10 carbon atoms in total.

In one embodiment, x is 1 to 4, for example x may be 1.

In one embodiment a), R¹ and R⁵ are both R^{w}, and R², R³ and R⁴ are all (Aa)ₓ:

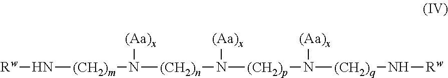

where R¹ and R⁵ are independently R^{w} as defined above and R², R³ and R⁴ are independently (Aa)ₓ as defined above. In such an embodiment, R¹ and R⁵ may, for example be the same R^{w} and R², R³ and R⁴ may, for example be the same (Aa)ₓ.

In another embodiment b), R² and R⁴ are R^{w}, R³ is hydrogen and R¹ and R⁵ are (Aa)ₓ:

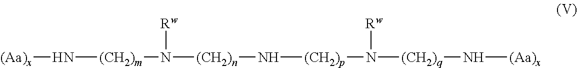

where R² and R⁴ are independently R^{w} as defined above and R¹ and R⁵ are independently (Aa)ₓ as defined above. In such an embodiment, R² and R⁴ may, for example be the same R^{w} and R¹ and R⁵ may, for example be the same (Aa)ₓ.

In another embodiment c), R² and R⁴ are R^{w}, and R¹, R³ and R⁵ are all hydrogen or all (Aa)ₓ:

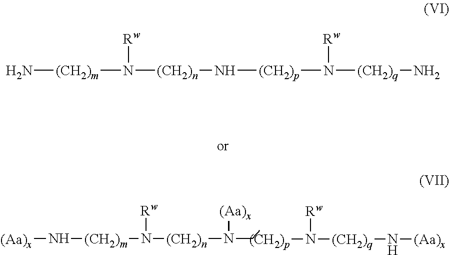

where R² and R⁴ are independently R^{w} as defined above and R¹, R³ and R⁵ are all H or all independently (Aa)ₓ as defined above. In such an embodiment, R² and R⁴ may, for example be the same R^{w}. R¹, R³ and R⁵ may, for example be the same (Aa).

In another embodiment d), R², R³ and R⁴ are R^{w}; and R¹ and R⁵ are both hydrogen or both (Aa)ₓ.

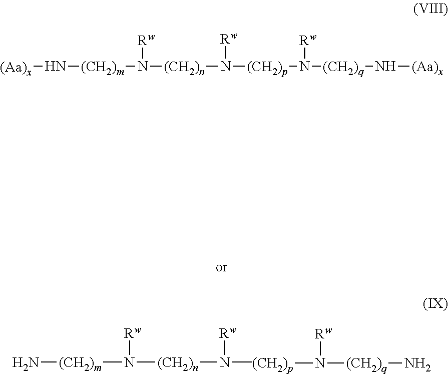

where R², R³ and R⁴ are R^{w} and R¹ and R⁵ are both hydrogen or both (Aa)ₓ as defined above. In such an embodiment, R², R³ and R⁴ may, for example be the same R^{w} and R¹ and R⁵ may, for example be the same (Aa)ₓ.

In a further embodiment the R^{w} saturated or unsaturated, branched or unbranched aliphatic carboxylic acid of up to 24 carbon atoms linked as its amide derivative has 10 or more carbon atoms, for example 12 or more, for example 14 or more, for example 16 or more carbon atoms. In a further embodiment the R^{w} saturated or unsaturated, branched or unbranched aliphatic carboxylic acid of up to 24 carbon atoms linked as its amide derivative is selected from:

### —C(O)(CH₂)₁₀CH₃

### —C(O)(CH₂)₁₂CH₃

### —C(O)(CH₂)₁₄CH₃

### —C(O)(CH₂)₁₆CH₃

### —C(O)(CH₂)₁₈CH₃

### —C(O)(CH₂)₂₀CH₃

—C(O)(CH₂)₇CH═CH(CH₂)₅CH₃ natural mixture
—C(O)(CH₂)₇CH═CH(CH₂)₇CH₃ natural mixture

### —C(O)(CH₂)₇CH═CH(CH₂)₅CH₃ Cis

### —C(O)(CH₂)₇CH═CH(CH₂)₇CH₃ Cis

### —C(O)(CH₂)₇CH═CH(CH₂)₅CH₃ Trans

### —C(O)(CH₂)₇CH═CH(CH₂)₇CH₃ Trans

### —C(O)(CH₂)₇CH═CHCH₂CH═CH(CH₂)₄CH₃

### —C(O)(CH₂)₇(CH═CHCH₂)₃CH₃

### —C(O)(CH₂)₃CH═CH(CH₂CH═CH)₃(CH₂)₄CH₃

### —C(O)(CH₂)₇CHCH(CH₂)₇CH₃

### —C(O)CH₂CH(CH₃)[CH₂CH₂CH₂CH(CH₃)]₃CH₃

### or —C(O)(CH₂)₂₂CH₃.

In one embodiment of the present invention the group is selected from —CO(CH₂)₇CH═CH(CH₂)₇CH₃ natural mixture, —CO(CH₂)₇CH═CH(CH₂)₇CH₃ Cis and —CO(CH₂)₇CH═CH(CH₂)₇CH₃ Trans.

Examples of pentamine gemini compounds, together with methods for preparing pentamine gemini compounds, are disclosed in WO06/053783.

Pentamine gemini compounds may be prepared from readily available starting materials using synthetic chemistry well known to the skilled person. The scheme shown in FIG. 12 shows a general scheme for the synthesis of an intermediate 5 for the synthesis of compounds of the invention.

As shown in the general scheme of FIG. 13, the intermediate 5 may be protected and reduced to give advanced pentamine intermediate 7 in which the R², R³ and R⁴ positions are protected and the R¹ and R⁵ positions are free NH₂ groups. By further reaction of the amino groups at R¹ and R⁵ positions to add R^{w} groups, and deprotection of the R², R³ and R⁴ positions followed by addition of (Aa)ₓ groups under appropriate conditions, molecules with the substitution pattern according to embodiment a) of the invention may be made.

As shown in the general scheme of FIG. 15, the intermediate 5 may be reduced to give a different advanced pentamine intermediate 12 in which only the R³ position is protected and the R¹, R², R⁴ and R⁵ positions are free amino groups. By subsequent protection of the primary amino groups at R¹ and R⁵ positions and addition of R^{w} groups at the R² and R⁴ positions followed by deprotection at R¹ and R⁵ and addition of (Aa)ₓ groups under appropriate conditions, and final deprotection at the R³ position molecules with the substitution pattern according to embodiment b) of the invention may be made. If the addition of groups (Aa)ₓ groups at the R¹ and R⁵ positions is omitted, molecules with the substitution pattern according to embodiment c) of the invention may be made in analogous fashion. If the deprotection at the R⁵ position occurs before addition of the (Aa)ₓ groups, molecules with the substitution pattern according to the second alternative of embodiment c) of the invention may be made in analogous fashion.

As shown in the general scheme of FIG. 16, the advanced intermediate 13, which may be made from intermediate 12, and which is protected at the R¹, R³ and R⁵ positions may be deprotected at the R³ position and subsequently functionalised by addition of an R^{w} group to each of the R², R³ and R⁴ positions. By subsequent deprotection of the amino groups at R¹ and R⁵ positions and addition of (Aa)ₓ groups under appropriate conditions, and final deprotection, molecules with the substitution pattern according to embodiment d) of the invention may be made. If the addition of groups (Aa)ₓ groups at the R¹ and R⁵ positions is omitted, molecules with the substitution pattern according to the alternative of embodiment d) of the invention with primary amino groups at the R¹ and R⁵ positions may be made in analogous fashion.

Various alternative protection and deprotection strategies are well known to the skilled person and suitable strategies may be devised for any particular desired final substitution pattern. For unsymmetric substitution patterns, physical separation of products or intermediates may be necessary. Suitable separation methods, for example chromatographic methods, are well known to the person skilled in the art.

Salts of molecules in accordance with the invention may be prepared by standard techniques, as shown for example in the schemes in FIGS. 17 and 18. In the scheme shown in FIG. 17, the salt formation step is also a deprotection step.

In another embodiment the gemini compound is a spermidine-based compound of formula (X):

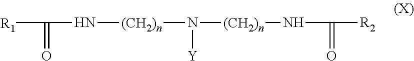

where Y is either: (Aa)ₓ or

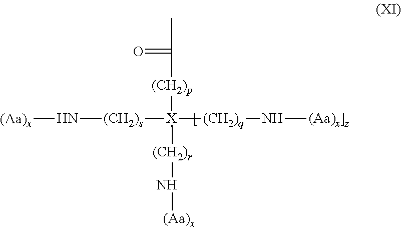

wherein
R₁ and R₂, which may be the same or different, is a saturated or unsaturated, linear or branched hydrocarbon chain of up to 24 carbon atoms;
m is 1 to 10;
n is 1 to 10;
(Aa)ₓ, which may be the same or different at each occurrence, is x natural or unnatural amino acids linked in a linear or branched manner,
x is 1 to 6;
p is 0 to 6;
q is 1 to 6;
r is 1 to 6;
s is 1 to 6;
X is N, CH or C, when X is N, z is 0 or 1, when X is CH, z is 0 when X is C, z is 1; or a salt, for example a pharmaceutically acceptable salt thereof.

In one embodiment m is 3 to 6, for example 4.

In another embodiment n is 3 to 6, for example 3.

In a further embodiment (Aa) is a basic amino acid. Examples of basic amino acids include [H₂N(CH₂)₃]₂N(CH₂)CO₂H, (H₂NCH₂)₂CHCO₂H, or L or D enantiomers of Ser, Lys, Orn, Dab (Diamino butyric acid) or Dap (diamino propionic acid). Examples of basic amino acids include amino acids comprising at least one NH₂ group (or optionally an OH group) in the side chain and comprising from 1 to 12, for example from 1 to 10 carbon atoms.

In a still further embodiment x is 1 to 4, for example 1.

In yet a further embodiment p is 1.

In another embodiment q is 1 or 3.

In still another embodiment r is 1 or 3.

In another embodiment s is 1 or 3.

In one embodiment a), Y is (Aa)ₓ. In such an embodiment, R₁ and R₂ may, for example, be the same.

In another embodiment b), Y is

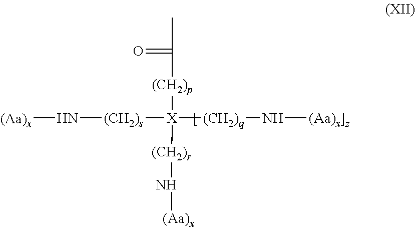

In such an embodiment X may for example be N. In such an embodiment, q, r and s may, for example, be the same and R₁ and R₂ may, for example, be the same. In one embodiment, X is N or C.

In a further embodiment the R₁ or R₂ saturated or unsaturated, linear or branched hydrocarbon chain of up to 24 carbon atoms has 10 or more carbon atoms, for example 12 or more, for example 14 or more, for example 16 or more carbon atoms. In a further embodiment the R₁ or R₂ saturated or unsaturated, linear or branched hydrocarbon chain of up to 24 carbon atoms is selected from:

### —(CH₂)₁₀CH₃

### —(CH₂)₁₂CH₃

### —(CH₂)₁₄CH₃

### —(CH₂)₁₆CH₃

### —(CH₂)₁₈CH₃

### —(CH₂)₂₀CH₃

—(CH₂)₇CH═CH(CH₂)₇CH₃ natural mixture
—(CH₂)₇CH═CH(CH₂)₅CH₃ natural mixture

### —(CH₂)₇CH═CH(CH₂)₅CH₃ Cis

### —(CH₂)₇CH═CH(CH₂)₇CH₃ Cis

### —(CH₂)₇CH═CH(CH₂)₅CH₃ Trans

### —(CH₂)₇CH═CH(CH₂)₇CH₃ Trans

### —(CH₂)₇CH═CHCH₂CH═CH(CH₂)₄CH₃

### —(CH₂)₇(CH═CHCH₂)₃CH₃

### —(CH₂)₃CH═CH(CH₂CH═CH)₃(CH₂)₄CH₃

### —(CH₂)₇CHCH(CH₂)₇CH₃

### —CH₂CH(CH₃)[CH₂CH₂CH₂CH(CH₃)]₃CH₃

### or —(CH₂)₂₂CH₃.

In one embodiment the hydrocarbon chain is selected from (CH₂)₇CH═CH(CH₂)₇CH₃ natural mixture, (CH₂)₇CH═CH(CH₂)₇CH₃ Cis and (CH₂)₇CH═CH(CH₂)₇CH₃ Trans.

Examples of spermidine gemini compounds, together with methods for preparing spermidine gemini compounds, are disclosed in WO06/053782.

Spermidine-based gemini compounds may be prepared from readily available starting materials using synthetic chemistry well known to the skilled person. FIG. 19 shows a general scheme for the synthesis of an intermediate 6 for the synthesis of compounds of the invention. FIG. 20 shows a general scheme for the synthesis of a protected example (Aa) group 9 for the synthesis of compounds of the invention. FIG. 21 shows a general scheme for the synthesis of a protected example (Aa) group 14 for the synthesis of compounds of the invention.

As shown in the general scheme of FIG. 22, reaction of the intermediate 6 by addition of (Aa)ₓ groups under appropriate conditions followed by deprotection produces molecules with the substitution pattern according to embodiment a) of the invention.

FIG. 23 shows a general scheme for the conversion of intermediate 6 to advanced intermediate 18 for the synthesis of compounds of the invention. As shown in the general scheme of FIG. 24, intermediate 18 may be used to produce molecules with the substitution pattern according to embodiment b) of the invention, by addition of (Aa)ₓ groups under appropriate conditions followed by deprotection.

Various alternative strategies are well known to the skilled person and suitable strategies may be devised for any particular desired final substitution pattern. For asymmetric substitution patterns, physical separation of products or intermediates may be necessary. Suitable separation methods, for example chromatographic methods, are well known to the person skilled in the art.

Salts of molecules in accordance with the invention may be prepared by standard techniques, as shown for example in the schemes in FIGS. 22 and 24. In the scheme shown in FIGS. 22 and 24, the salt formation step is also a deprotection step.

In another embodiment the gemini compound is an ester-linked gemini compound of formula (XIII):

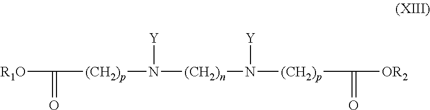

where Y is either H or (Aa)ₓ where (Aa) is a basic amino acid and x is 1 to 6;
R₁ and R₂, which may be the same or different, is a saturated or unsaturated, linear or branched hydrocarbon chain of up to 24 carbon atoms;
n is 1 to 10; and
p is 1 to 6;
or a pharmaceutically acceptable salt thereof.

In one embodiment the R₁ or R₂ saturated or unsaturated, linear or branched hydrocarbon chain of up to 24 carbon atoms has 10 or more carbon atoms, for example 12 or more, for example 14 or more, for example 16 or more carbon atoms.

In a further embodiment the R₁ or R₂ saturated or unsaturated, linear or branched hydrocarbon chain of up to 24 carbon atoms is selected from:

### —(CH₂)₁₀CH₃

### —(CH₂)₁₂CH₃

### —(CH₂)₁₄CH₃

### —(CH₂)₁₆CH₃

### —(CH₂)₁₈CH₃

### —(CH₂)₂₀CH₃

—(CH₂)₇CH═CH(CH₂)₇CH₃ natural mixture
—(CH₂)₇CH═CH(CH₂)₅CH₃ natural mixture

### —(CH₂)₇CH═CH(CH₂)₅CH₃ Cis

### —(CH₂)₇CH═CH(CH₂)₇CH₃ Cis

### —(CH₂)₇CH═CH(CH₂)₅CH₃ Trans

### —(CH₂)₇CH═CH(CH₂)₇CH₃ Trans

### —(CH₂)₇CH═CHCH₂CH═CH(CH₂)₄CH₃

### —(CH₂)₇(CH═CHCH₂)₃CH₃

### —(CH₂)₃CH═CH(CH₂CH═CH)₃(CH₂)₄CH₃

### —(CH₂)₇CHCH(CH₂)₇CH₃

### —CH₂CH(CH₃)[CH₂CH₂CH₂CH(CH₃)]₃CH₃

### or —(CH₂)₂₂CH₃.

In one embodiment of the present invention, the hydrocarbon chain is selected from (CH₂)₇CH═CH(CH₂)₇CH₃ natural mixture, (CH₂)₇CH═CH(CH₂)₇CH₃ Cis and (CH₂)₇CH═CH(CH₂)₇CH₃ Trans.

In one embodiment n is 3 to 6. for example n may be 4.

In a further embodiment p is 1 to 4. for example p may be 2.

In a still further embodiment Y is (Aa).

(Aa)ₓ, which may be the same or different at each occurrence, is x natural or unnatural amino acids linked in a linear or branched manner; (Aa) is a basic amino acid, for example, L or D enantiomers of serine (ser), lysine (lys), ornithine (orn), diaminobutyric acid (dab) or diaminopropionic acid (dap).

x is 1 to 6; for example 1 to 3. for example x may be 1.

The group (Aa) is linked to the N in formula (I) by means of a peptide (amide) bond between the N and the carboxy group on the amino acid residue.

In a one embodiment the ester gemini is selected from the group consisting of:

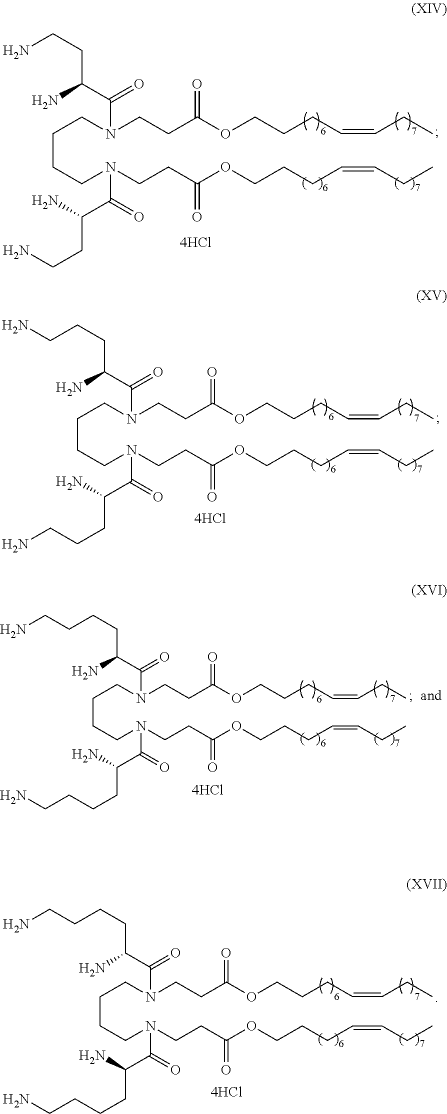

Examples of ester-linked gemini compounds, together with methods for preparing ester-linked gemini compounds, are disclosed in PCT/EP2006/000998.

Ester-linked gemini compounds may be prepared from readily available starting materials using synthetic chemistry well known to the skilled person. FIG. 25 shows a general scheme for the synthesis of compounds of the invention where p=2. For compounds where p=1 or 3 to 6 other dicarboxylic acids (intermediate 3 in FIG. 25) may be used as starting materials using techniques well known in the art, for example Jaine, N et al.; Journal of Inorganic Biochemistry 1994, 53(2), 79-94 for where p=1; Reppe et al.; JLACBF; Justus Liebigs Ann. Chem.; 596; 1955; 1,215 for where p=3 and Gautier; Renault; Recl. Trav. Chim. Pays-Bas; 69; 1950; 421, 426 for where p=4.

Various alternative strategies are well known to the skilled person and suitable strategies may be devised for any particular desired final substitution pattern. For asymmetric substitution patterns, physical separation of products or intermediates may be necessary. Suitable separation methods, for example chromatographic methods, are well known to the person skilled in the art.

Salts of ester-linked gemini molecules may be prepared by standard techniques.

In another embodiment the gemini compound is selected from the group consisting of:

GSC103-L-Lysine

GSC103-L-Lysine, oleic acid/stearic acid;

GSC103-D-Lysine, oleic acid/stearic acid;

GSC103-L-Diaminobutyric acid, oleic acid, oleic acid;

GSC103-L-Ornithine, oleic acid, stearic acid;

GSC170-Lysine;

GSC170-Ornithine;

GS064A;

GS062A;

GS092A; and

GS543A.

In a further embodiment the polynucleotide vaccine may further comprise one or more supplements which may increase the efficiency of transfection of the polynucleotide of the polynucleotide vaccine into the target cell and/or increase the adjuvant effect of the gemini compound. Such supplements may be selected from, for example:

(i) a neutral carrier, for example dioleyl phosphatidylethanolamine (DOPE) (Farhood, H., et al (1985) Biochim. Biophys. Acta 1235 289);

(ii) a complexing reagent, for example the commercially available PLUS™ reagent (Life Technologies Inc. Maryland, USA); or

(iii) peptides, such as polylysine or polyornithine peptides or peptides comprising primarily, but not exclusively, basic amino acids such as lysine, ornithine and/or arginine (see for example Henner, W. D et al (1973) J. Virol. 12(4) pp 741-747). The list above is not intended to be exhaustive and other supplements that increase the efficiency of transfection and/or adjuvant effect of the gemini are taken to fall within the scope of the invention.

It is possible for the vaccination methods and compositions according to the present application to be adapted for protection or treatment of mammals against a variety of disease states such as, for example, viral, bacterial or parasitic infections, cancer, allergies and autoimmune disorders.

The polynucleotide sequences referred to in this application which are to be expressed within a mammalian system in order to induce an antigenic response, may encode for an entire protein, or merely a shorter peptide sequence that is capable of initiating an antigenic response. Throughout this specification and the appended claims, the phrase “antigenic peptide” or “immunogen” is intended to encompass all peptide or protein sequences which are capable of inducing an immune response within the animal concerned. In one embodiment of the present invention, however, the polynucleotide sequence will encode for a full protein that is associated with the disease state, as the expression of full proteins within the animal system is more likely to mimic natural antigen presentation, and thereby evoke a full immune response.

Antigens which are capable of eliciting an immune response against a human pathogen include those in which the antigen or antigenic composition is derived from any of a range of viral, bacterial, parasitic and yeast sources. Viral antigen sources include: HIV-1 (such as gag, p17, p24, p41, p40, nef, pol, RT, p66, env, gp120 or gp160, gp40, p24, gag, vif, vpr, vpu, rev, tat), or combinations thereof, for example a combination of gag, RT and Nef as described in WO03/025003; human herpes viruses (such as gH, gL gM gB gC gK gE or gD or derivatives thereof, or Immediate Early proteins such as ICP27, ICP 47, IC P 4, ICP36 from HSV1 or HSV2); cytomegalovirus, especially human (such as gB or derivatives thereof); Epstein Barr virus (such as gp350 or derivatives thereof); Varicella Zoster Virus (such as gpI, II, III and IE63); hepatitis viruses such as hepatitis B virus (for example hepatitis B surface antigen or hepatitis core antigen or pol) or hepatitis C and hepatitis E virus antigens; and other viral pathogens such as the paramyxoviruses, including Respiratory Syncytial virus (such as F and G proteins or derivatives thereof), parainfluenza virus, measles virus, mumps virus, human papilloma viruses (for example HPV6, 11, 16, 18, and the antigens L1, L2, E1, E2, E3, E4, E5, E6, E7), flaviviruses (e.g. Yellow Fever Virus, Dengue Virus, Tick-borne encephalitis virus, Japanese Encephalitis Virus) and Influenza virus cells (such as HA, NP, NA, or M proteins, or combinations thereof). Bacterial sources include: Neisseria spp. such as N. gonorrhea and N. meningitidis (e.g. transferrin-binding proteins, lactoferrin binding proteins, PilC, adhesins); S. pyogenes (for example M proteins or fragments thereof, or C5A protease); S. agalactiae, S. mutans; H. ducreyi; Moraxella spp. such as M. catarrhalis (also known as Branhamella catarrhalis; antigens include high and low molecular weight adhesins and invasins); Bordetella spp., including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, 85B or 85C, MPT 44, MPT59, MPT45, HSP10, HSP65, HSP70, HSP 75, HSP90, PPD 19 kDa [Rv3763], PPD 38 kDa [Rv0934]), M. bovis, M. leprae, M. avium, M. paratuberculosis and M. smegmatis; Legionella spp., including L. pneumophila; Escherichia spp., including enterotoxic E. coli (for example colonization factors, heat-labile toxin or derivatives thereof, heat-stable toxin or derivatives thereof), enterohemorragic E. coli and enteropathogenic E. coli (for example shiga toxin-like toxin or derivatives thereof); Vibrio spp., including V. cholera (for example cholera toxin or derivatives thereof); Shigella spp., including S. sonnei, S. dysenteriae and S. flexnerii; Yersinia spp., including Y. enterocolitica (for example a Yop protein), Y. pestis and Y. pseudotuberculosis; Campylobacter spp., including C. jejuni (for example toxins, adhesins and invasins) and C. coli; Salmonella spp., including S. typhi, S. paratyphi, S. choleraesuis and S. enteritidis; Listeria spp., including L. monocytogenes; Helicobacter spp., including H. pylori (for example urease, catalase, vacuolating toxin); Pseudomonas spp., including P. aeruginosa; Staphylococcus spp., including S. aureus and S. epidermidis; Enterococcus spp., including E. faecalis and E. faecium; Clostridium spp., including C. tetani (for example tetanus toxin and derivatives thereof), C. botulinum (for example botulinum toxin and derivatives thereof), and C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), and B. hermsii; Ehrlichia spp., including E. equi and the agent of the Human Granulocytic Ehrlichiosis; Rickettsia spp., including R. rickettsii; Chlamydia spp., including C. trachomatis (for example MOMP, heparin-binding proteins), C. pneumoniae (for example MOMP, heparin-binding proteins), and C. psittaci; Leptospira spp., including L. interrogans; and Treponema spp., including T. pallidum (for example the rare outer membrane proteins), T. denticola, and T. hyodysenteriae. Parasitic sources include: Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major, Pneumocystis spp., including P. carinii; Trichomonas spp., including T. vaginalis; and Schisostoma spp., including S. mansoni. Yeast sources include: Candida spp., including C. albicans; and Cryptococcus spp., including C. neoformans.
(i) Other examples of specific antigens for M. tuberculosis are, for example, Rv2557, Rv2558, RPFs: Rv0837c, Rv1884c, Rv2389c, Rv2450, Rv1009, aceA (Rv0467), PstS1, (Rv0932), SodA (Rv3846), Rv2031c 16 kDal., Tb Ra12, Tb H9, Tb Ra35, Tb38-1, Erd 14, DPV, MTI, MSL, mTTC2 and hTCC1 (WO 99/51748). Proteins for M. tuberculosis also include fusion proteins and variants thereof in which at least two, or in which at least three, polypeptides of M. tuberculosis are fused into a larger protein. Examples of such fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, and TbH9-DPV-MTI (WO 99/51748).
(ii) Examples of antigens for Chlamydia include, for example, the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Examples of bacterial antigens derived from Streptococcus spp., including S. pneumoniae (e.g. PsaA, PspA, streptolysin, choline-binding proteins) and the protein antigen Pneumolysin (Biochem Biophys Acta, 1989, 67, 1007; Rubins, J. B. et al. (1998) Microbial Pathogenesis 25: 337-42), and mutant detoxified derivatives thereof (WO 90/06951; WO 99/03884), PhtD, PhtA, PhtB, PhtE and CbpA. Other examples of antigens derived from Haemophilus spp., including H. influenzae type B (for example PRP and conjugates thereof), non typeable H. influenzae (for example OMP26, high molecular weight adhesins, P5, P6, protein D and lipoprotein D, and fimbrin and fimbrin derived peptides (U.S. Pat. No. 5,843,464) or multiple copy variants or fusion proteins thereof).

The antigens that may be used in the present invention may further comprise antigens derived from parasites that cause malaria. For example, antigens from Plasmodium falciparum include RTS,S and TRAP. RTS is a hybrid protein comprising substantially all the C-terminal portion of the circumsporozoite (CS) protein of P. falciparum linked via four amino acids of the preS2 portion of hepatitis B surface antigen to the surface (S) antigen of hepatitis B virus. Its full structure is disclosed in the International Patent Application No. PCT/EP92/02591, published under Number WO 93/10152 claiming priority from UK patent application No. 9124390.7. When expressed in yeast RTS is produced as a lipoprotein particle, and when it is co-expressed with the S antigen from HBV it produces a mixed particle known as RTS,S. TRAP antigens are described in the International Patent Application No. PCT/GB89/00895, published under WO 90/01496. In one embodiment of the present invention is a malaria vaccine wherein the antigenic preparation comprises a combination of the RTS,S and TRAP antigens. Other plasmodia antigens that are likely candidates to be components of a multistage malaria vaccine are P. falciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in other Plasmodium spp.

The invention contemplates the use of an anti-tumour antigen and may be useful for the immunotherapeutic treatment of cancers. For example, tumour rejection antigens such as those for prostrate, breast, colorectal, lung, pancreatic, renal or melanoma cancers. Exemplary antigens include MAGE 1, MAGE 3 and MAGE 4, or other MAGE antigens such as disclosed in WO99/40188, PRAME, BAGE, Lage (also known as NY Eos 1) SAGE and HAGE (WO 99/53061) or GAGE (Robbins, P. F. & Kawakami, Y. (1996) Current Opinion in Immunology 8: 628-36; Van den Eynde, B. J. & Boon, T. (1997) International Journal of Clinical and Laboratory Research 27: 81-6. Correale, P. et al. (1997) Journal of the National Cancer Institute 89: 293-300). Indeed these antigens are expressed in a wide range of tumour types including melanoma, lung carcinoma, sarcoma and bladder carcinoma.

MAGE antigens for use in the present invention may be expressed as a fusion protein with an expression enhancer or an immunological fusion partner. In particular, the MAGE protein may be fused to Protein D from Haemophilus influenzae B. In particular, the fusion partner may comprise the first one third of Protein D. Such constructs are disclosed in WO 99/40188. Other examples of fusion proteins that may contain cancer specific epitopes include bcr/abl fusion proteins.

In one embodiment prostate antigens are utilised, such as Prostate Specific Antigen (PSA), PAP, PSCA (Reiter, R. E. et al. (1998) PNAS USA 95: 1735-40), PSMA or the antigen known as Prostase. Prostase is a prostate-specific serine protease (trypsin-like), and has been described by Nelson, P. S. et al. (1999; PNAS USA 96: 3114-9). The nucleotide sequence and deduced polypeptide sequence of the mature protein, and homologues, are disclosed in (PNAS USA (1999) 96: 3114-9) and in International Patent Applications WO 98/12302 (and also the corresponding granted U.S. Pat. No. 5,955,306), WO 98/20117 (and also the corresponding granted U.S. Pat. No. 5,840,871 and U.S. Pat. No. 5,786,148) (prostate-specific kallikrein) and WO 00/04149 (P703P).

The present invention provides antigens comprising prostate protein fusions based on prostate protein and fragments and homologues thereof (“derivatives”). Such derivatives are suitable for use in therapeutic vaccine formulations that are suitable for the treatment of prostate tumours. Typically the fragment will contain at least 20, for example 50, or for example 100, contiguous amino acids as disclosed in the above referenced patent and patent applications.

A further example of a prostate antigen is known as P501S, sequence ID No. 113 of WO98/37814. Immunogenic fragments and portions encoded by the gene thereof comprising at least 20, for example 50, or for example 100, contiguous amino acids as disclosed in the above referenced patent application, are contemplated. A particular fragment is PS108 (WO 98/50567).

Other prostate specific antigens are known from WO98/37418, and WO/004149. Another is STEAP (Hubert, R. S. et al. (1999) PNAS USA 96: 14523-8).

Other tumour associated antigens useful in the context of the present invention include: Plu-1 (Lu, P. J. et al. (1999) Journal of Biological Chemistry 274: 15633-45), HASH-1, HasH-2, Cripto (Salomon, D. S. et al. (1999) Bioessays 21: 61-70; U.S. Pat. No. 5,654,140), and Criptin (U.S. Pat. No. 5,981,215). Additionally, antigens particularly relevant for vaccines in the therapy of cancer also comprise tyrosinase and survivin.

The present invention is also useful in combination with breast cancer antigens such as Muc-1, Muc-2, EpCAM, HER 2/Neu, mammaglobin (U.S. Pat. No. 5,668,267) or those disclosed in WO 00/52165, WO99/33869, WO99/19479, WO 98/45328. HER/2 neu antigens are disclosed, inter alia, in U.S. Pat. No. 5,801,005. In one embodiment the HER/2 neu comprises the entire extracellular domain (comprising approximately amino acids 1-645), or fragments thereof, and at least an immunogenic portion of or the entire intracellular domain (approximately the 580 C-terminal amino acids). In particular, the intracellular portion should comprise the phosphorylation domain or fragments thereof. Such constructs are disclosed in WO00/44899. Examples of such constructs include a construct which is known as ECD PD; a second is known as ECD ΔPD (see WO/00/44899). The HER/2 neu as used herein can be derived from rat, mouse or human.

The antigens may also be associated with tumour-support mechanisms (e.g. angiogenesis, tumour invasion), for example tie 2.

Vaccines of the present invention may also be used for the prophylaxis or therapy of chronic disorders in addition to allergy, cancer or infectious diseases. Such chronic disorders are diseases such as asthma, atherosclerosis, and Alzheimer's and other auto-immune disorders. Vaccines for use as a contraceptive may also be considered.

Antigens relevant for the prophylaxis and the therapy of patients susceptible to or suffering from Alzheimer's neurodegenerative disease are, in particular, the N-terminal 39-43 amino acid fragment of the β-amyloid precursor protein and smaller fragments). This antigen is disclosed in the International Patent Application No. WO 99/27944 (Athena Neurosciences).

Potential self-antigens that could be included as vaccines for auto-immune disorders or as a contraceptive vaccine include: cytokines, hormones, growth factors or extracellular proteins, for example a 4-helical cytokine, for example IL13. Cytokines include, for example, IL1, IL2, IL3, IL4, IL5, IL6, IL7, IL8, IL9, IL10, IL11, IL12, IL13, IL14, IL15, IL16, IL17, IL18, IL20, IL21, TNF, TGF, GM-CSF, MCSF and OSM. 4-helical cytokines include IL2, IL3, IL4, IL5, IL13, GM-CSF and MCSF. Hormones include, for example, luteinising hormone (LH), follicle stimulating hormone (FSH), chorionic gonadotropin (CG), VGF, GHrelin, agouti, agouti related protein and neuropeptide Y.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as tuberculosis (TB), HIV infections such as AIDS, and hepatitis B (HepB) virus infections.

The polynucleotides encoding such antigens, may encode immunogenic derivatives or immunogenic fragments thereof rather than the whole antigen.

It will be understood that for all of the polynucleotide sequences included in the invention, these do not necessarily represent sequences encoding the full length or native proteins. Immunogenic derivatives such as truncated or otherwise altered e.g. mutated proteins are also contemplated, as are fragments which encode at least one epitope, for example a CTL epitope, typically a peptide of at least 8 amino acids. Polynucleotides which encode a fragment of at least 8, for example 8-10 amino acids or up to 20, 50, 60, 70, 100, 150 or 200 amino acids in length are considered to fall within the scope of the invention as long as the encoded oligo or polypeptide demonstrates antigenicity, that is to say that the major CTL epitopes are retained by the oligo or polypeptide.

The polypeptide molecules encoded by the polynucleotide sequences according to the invention may represent a fragment of for example 50% of the length of the native protein, which fragment may contain mutations but which retains at least one epitope and demonstrates antigenicity. Similarly, immunogenic derivatives according to the invention must demonstrate antigenicity. Immunogenic derivatives may provide some potential advantage over the native protein such as reduction or removal of a function of the native protein which is undesirable in a vaccine antigen such as enzyme activity (for example, RT), or CD4 downregulation (for example, Nef).

The polynucleotide sequences may be codon optimised for mammalian cells. Such codon-optimisation is described in detail in WO05/025614.

In one embodiment of the present invention the constructs comprise an N-terminal leader sequence. The signal sequence, transmembrane domain and cytoplasmic domain are individually all optionally present or deleted. In one embodiment of the present invention all these regions are present but modified.

The polynucleotide of the vaccines and the adjuvants of the present invention may be administered simultaneously or separately. For instance the polynucleotide and the adjuvant may be co-formulated in a single composition, or alternatively may be separately formulated in distinct compositions. In the latter instance the at least two compositions are administered in functional cooperation, and may be administered at substantially the same time, or alternatively be administered at different time points separated by, in different embodiments, within 30 minutes to 1 hour apart, or within 1 and 2 hours apart, or within 12-36 hours apart, such as 24 hours apart; or the two compositions may, substantially, be administered the next following day. When the at least two compositions are administered on different occasions, the polynucleotide may be administered before the adjuvant.

Accordingly, there is provided a kit comprising two compositions, the polynucleotide containing composition and the adjuvant containing composition, for separate or simultaneous administration. In this context the separate administration may be separated by administration site or time, or both. The kit may also include instructions to administer the two compositions simultaneously or separately.

In one embodiment of the present invention the plasmids of the vaccines are prevented from replicating within the mammalian vaccinee and integrating within the chromosomal DNA of the host, the plasmid will for example be produced without an origin of replication that is functional in eukaryotic cells.

The immunogen component comprising a vector which comprises the nucleotide sequence encoding an antigenic peptide can be administered in a variety of manners. It is possible for the vector to be administered in a naked form (that is, as a naked nucleotide sequence not in association with liposomal formulations, with viral vectors or transfection facilitating proteins) suspended in an appropriate medium, for example a buffered saline solution such as PBS, and then injected intramuscularly, subcutaneously, intraperitoneally or intravenously (some earlier data suggests that intramuscular or subcutaneous injection is preferable; Brohm, et al. (1998) Vaccine 16: 949-54, the disclosure of which is included herein in its entirety by way of reference). It is additionally possible for the vectors to be encapsulated by, for example, liposomes or within polylactide co-glycolide (PLG) particles for administration via the oral, nasal or pulmonary routes in addition to the routes detailed above.

It is also possible, according to a one embodiment of the invention, for intradermal administration of the immunogen component, for example via use of gene-gun (particularly particle bombardment) administration techniques. Such techniques may involve coating of the immunogen component on to dense micro-beads, such as gold beads, which are then administered under high pressure into the epidermis, such as, for example, as described in Haynes, J. R. et al. (1996; Journal of Biotechnology 44: 37-42). In this context the adjuvant component may be co-formulated on the dense microbeads, or on separate populations of microbeads, or alternatively the polynucleotide vaccine may be administered ballistically on microbeads and the adjuvant administered separately via systemic or local delivery, possibly at the site of polynucleotide delivery by intradermal or subcutaneous injection.

In an alternative embodiment of the present invention, there is provided a patch comprising a plurality of needles, being in the range of 30-1000 micrometers in length, the external surface of which is coated with a solid reservoir medium. The solid reservoir medium in this context would comprise the vaccines of the present invention in solid form. Microneedles of this form are described in WO 02/07813 and WO 03/061636, the contents of which are incorporated herein, and in particular the claims thereof are intended to be read, with the addition of gemini surfactant in the context of this disclosure.

The adjuvants and vaccines of the present invention may be administered via a variety of different administration routes, such as intramuscular, subcutaneous, intraperitoneal, intradermal, or topical routes. The adjuvant or polynucleotide components may be administered via the subcutaneous, intradermal or topical routes. In one embodiment, the administration of both components, the polynucleotide and adjuvant, is by the same route. In another embodiment, the polynucleotide is administered by ballistic delivery (gene gun) into the epidermis or dermis, and the adjuvant composition is delivered in the vicinity of the polynucleotide either topically or by intradermal or subcutaneous injection.

The dose of administration of the adjuvant will also vary, but may, for example, range in a liquid form of the vaccine between about 5 μg per ml to about 5 mg per ml, and may be between 25 μg per ml to about 1 mg per ml, and may be between 50 to 500 μg per ml. In a liquid form between 0.5 and 1 ml of the vaccine may be administered to a human vaccinee.

In a dry form of the vaccine a total mass of the adjuvant may also be in the range of 5 μg to about 5 mg per dose, and may be between 25 μg to about 1 mg per dose, and may be between 50 to 500 μg per dose.

Administration of the adjuvant may be repeated with each subsequent or booster administration of the nucleotide sequence.

Administration of the pharmaceutical composition may take the form of one or of more than one individual dose, for example as repeat doses of the same polynucleotide-containing gemini, or in a heterologous “prime-boost” vaccination regime wherein the compositions of the invention are administered in a schedule with other suitable forms of administration, for example use of viral vectors such as adenoviral vectors, or for example by administration of naked DNA. A heterologous prime-boost regime uses administration of different forms of vaccine in the prime and the boost, each of which may itself include two or more administrations. The priming composition and the boosting composition will have at least one antigen in common, although it is not necessarily an identical form of the antigen, it may be a different form of the same antigen.

A prime boost regime of use with the immunogenic compositions of the present invention may take the form of a heterologous polynucleotide-containing gemini surfactant and polynucleotide-containing adenoviral vector or naked DNA prime boost, for example, a polynucleotide-containing gemini surfactant priming dose, followed by an adenoviral vector boost, or for example, an adenoviral vector prime or naked DNA prime followed by one or more polynucleotide-containing gemini surfactant boosts. Such polynucleotide-containing gemini surfactant or DNA priming or boosting doses may be delivered by intra-muscular or intra-dermal administration of DNA, or by particle acceleration techniques. Alternatively such a prime boost regime could comprise for example a protein dose and gemini surfactant dose according to the present invention, with the priming dose comprising the protein, and the boosting dose comprising the polynucleotide-containing gemini surfactant or for example wherein the priming dose comprises a polynucleotide-containing gemini surfactant and the boosting dose comprises a protein.

The dose of the polynucleotide encoding the antigen will depend on the route of administration and will be readily determined by the man skilled in the art. Conventionally speaking for gene gun applications the dose will be between 0.5 and 100 μg per administration, and for intramuscular administration of “naked” DNA between 10 and 2000 μg per administration.

Uses of the word “comprising” in the disclosures throughout this specification are intended to be read in both senses of the word, in that in the context of each disclosure of elements the word “comprising” should be read in its inclusive sense, in that other elements may also be included, and also in its exclusive sense in that the disclosure is restricted to those elements, and therefore each word comprising may be substituted with the word “consisting”.

The present invention is exemplified by, and not limited to, the following examples.

### 1) Unsymmetrical Spermine-Based Geminis

### Description 1

### 2,2,2-Trifluoro-N-(3-{4-[3-(2,2,2-trifluoro-acetylamino)-propylamino]-butylamino}-propyl)-acetamide trifluoroacetic acid salt

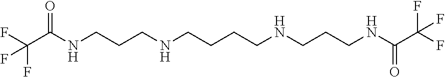

To a stirring solution of spermine dihydrate (10.0 g, 41.9 mmol) in acetonitrile (200 mL) was added ethyl trifluoroacetate (29.8 g, 209 mmol) and water (1.0 ml). The reaction mixture was heated at reflux for 18 h, then allowed to cool to room temperature and the solvent evaporated in vacuo. The residual solid was triturated with CH₂Cl₂ (2×100 mL) to give the bis(trifluoroacetamide) as a white solid (23.0 g, 88%).

LC-MS (ESI): t_{R}=1.213 min (m/z=395.2 [M+H]⁺).

### Description 2

### (4-{tert-Butoxycarbonyl-[3-(2,2,2-trifluoro-acetylamino)-propyl]-amino}-butyl)-[3-2,2,2-trifluoro-acetylamino)-propyl]-carbamic acid tert-butyl ester

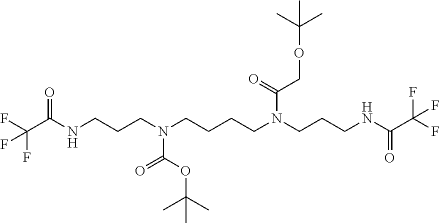

To a stirring solution of the bis-trifluoroacetate salt (description 1) (16.5 g, 26.6 mmol) and diisopropylethylamine (13.9 mL, 79.7 mmol) in THF (165 mL) was added di-tert-butyl dicarbonate (12.2 g, 55.8 mmol). The mixture was stirred at room temperature for 16 h, then concentrated in vacuo and diluted with EtOAc (300 mL). The organic solution was washed with 5% aq. NaHCO₃ (2×80 mL), 5% aq. KHSO₄ (2×80 mL), and brine (2×80 mL), dried (Na₂SO₄) and then evaporated in vacuo to leave the bis-carbamate as a white solid (11.4 g, 72%); mp 108-110° C. (from diethyl ether).

### Description 3

### (3-Aminopropyl)-{4-[(3-aminopropyl)-tert-butoxycarbonyl-amino]-butyl}-carbamic acid tert-butyl ester

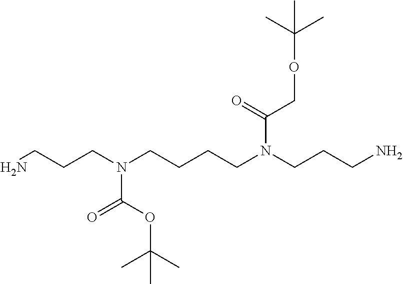

A stirring mixture of the trifluoroacetamide (description 2) (28.6 g, 48.1 mmol), potassium carbonate (33.2 g, 240 mmol), and water (100 mL) in methanol (600 mL) was heated at reflux for 2 h. The mixture was cooled to −10° C. and filtered and then concentrated in vacuo. The residue was diluted with EtOAc (500 mL) and methanol (100 mL) and the organic solution was washed with brine (1×100 mL). The aqueous washings were basified with 5% aq. sodium hydroxide extracted with chloroform (4×100 mL). All the organic phases were combined, washed with brine (1×100 mL), dried (Na₂SO₄) and evaporated in vacuo to leave the title amine as a pale yellow gum (16.4 g, 85%).

TLC (SiO₂): R_{f}=0.50 [90:10] EtOH:0.88 NH₃.

### Description 4

### Preparation of Oleic Acid Pentafluorophenyl Ester

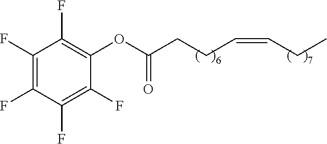

A mixture of pentafluorophenol (50.0 g, 271 mmol) and trifluoroacetic anhydride (85.0 g, 404 mmol) was stirred at 40° C. for 18 h. The resulting mixture was fractionally distilled to afford pentafluorophenol trifluoroacetate as a colourless liquid (75.2 g, 99%); by 122-125° C.

A solution of oleic acid (30.0 g, 106 mmol) in anhydrous DMF (100 mL) was added to a solution of pentafluorophenol trifluoroacetate (32.7 g, 116 mmol) in anhydrous DMF (100 mL), followed slowly by pyridine (9.16 g, 116 mmol). The resulting mixture was stirred at room temperature for 18 h, then diluted with ethyl acetate (200 mL) and washed successively with 0.1N hydrochloric acid (1×100 mL), saturated aq. sodium bicarbonate solution (1×100 mL), and brine (1×50 mL). The organic solution was dried (MgSO₄) and concentrated in vacuo to leave oleic acid pentafluoroacetate as a colourless viscous liquid (45.0 g, 95%).

### Description 5

### (3-Amino-propyl)-{4-[tert-butoxycarbonyl-(3-octadec-9-enoylamino-propyl)-amino]-butyl}-carbamic acid tert-butyl ester

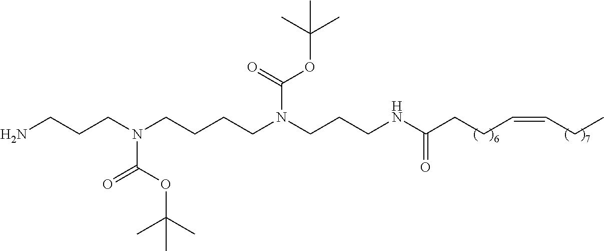

A stirring solution of the diamine of description 3 (3.50 g, 8.69 mmol) and triethylamine (5.26 mL, 38.0 mmol) in dichloromethane was maintained at −78° C. and treated dropwise over 3 h with a solution of oleic acid pentafluorophenyl ester (3.89 g, 8.69 mmol) in dichloromethane (100 mL). After completion of the addition, the cooling bath was left in place and the temperature was allowed to warm to room temperature with stirring over 18 h. The solvent was evaporated to leave a brown gum which was purified by column chromatography over silica gel eluting with a mixture of ethyl acetate:methanol:ammonia (98:2:2) to afford the desired monoacylated carbamate as a colourless gum (3.08 g, 53%).

TLC (SiO₂): R_{f}=0.25 [96:2:2] EtOAc:MeOH:0.88 NH₃.

### Description 6

### Preparation of Unsymmetric Carboxamide

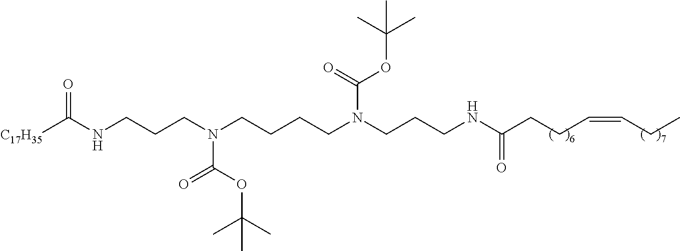

A mixture of stearic acid (255 mg, 0.90 mmol), TBTU (288 mg, 0.90 mmol), and HOBt (121 mg, 0.90 mmol) was stirred in dichloromethane (18 mL) at room temperature for 5 min. A solution of the amine of description 5 (0.50 g, 0.79 mmol) in dichloromethane (5.0 mL) was added followed by diisopropylethylamine (0.78 mL, 4.50 mmol) and stirring was continued for 18 h. The organic solution was washed with 5% aq. citric acid (5 mL), 2M aq. sodium carbonate solution (10 mL), and water (10 mL), then dried (MgSO₄) and concentrated in vacuo to leave the title carboxamide as a colourless gum (526 mg, 71%).

### Description 7

### Preparation of the Unsymmetric Amine Hydrochloride

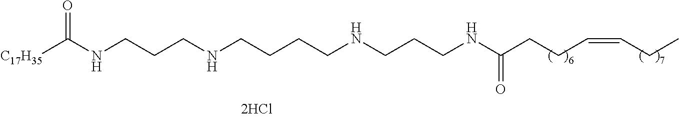

The carbamate of description 6 (520 mg, 0.56 mmol) was dissolved in diethyl ether (10 mL) and treated at room temperature with a solution of HCl in diethyl ether (2M, 5 mL) with stirring. After 2 h, the solvent was evaporated under a nitrogen stream to a precipitate which was washed with diethyl ether (2×10 mL), and then dried in vacuo to leave the title amine hydrochloride as a white solid (410 mg, quantitative).

### Description 8

### General Procedure for the Preparation of the Gemini Surfactant Hydrochlorides of Examples 1 to 3

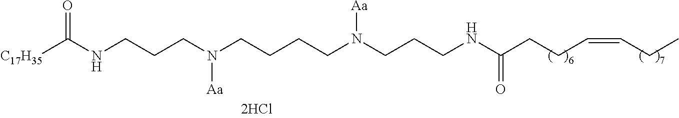

A stirring mixture of the amine hydrochloride of description 7 (80 mg, 0.11 mmol), TBTU (75 mg, 0.23 mmol), HOBt (32 mg, 0.23 mmol) and the Boc protected amino acid (0.23 mmol) in dichloromethane (10 mL) was treated with diisopropylethylamine (0.10 mL, 0.60 mmol) at room temperature. The mixture was diluted with additional dichloromethane (10 mL) and washed with 1M aq. citric acid (5 mL), 5% aq, sodium bicarbonate (5 mL) and brine (5 mL), then dried (Na₂SO₄) and the organic solution was concentrated in vacuo to leave the carbamate as a gum.

The gum was dissolved in diethyl ether (10 mL) and treated with a solution of HCl in diethyl ether (2M, 5 mL). The mixture was stirred at room temperature for 2 h and then the solvent was evaporated in a nitrogen stream. The residual solid was washed with further diethyl ether (2×5 mL), dried in vacuo, and then purified by reverse phase HPLC eluting with a mixture of acetonitrile in water (5-95%) containing 0.1% TFA to afford the trifluoroacetic acid salt as a white solid. The solid was dissolved in a solution of HCl in dioxane (4M, 2 mL) and then concentrated in vacuo to leave a solid which was triturated with diethyl ether (2×5 mL) to afford the Gemini surfactant hydrochloride salt as a white solid (20-35%).

### Example 1

### GSC103 L-Lys, Oleic Acid, Stearic Acid

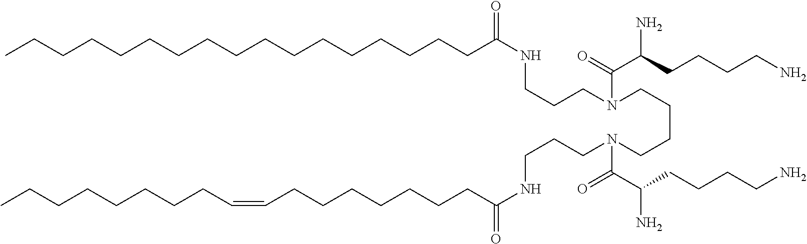

HRMS (ESI): t_{R}=11.74 min; m/z calcd (C₅₈H₁₁₇N₈O₄) 989.9198, found 989.9199 [M+H]⁺.

### Example 2

### GSC103 D-Lys, Oleic Acid, Stearic Acid

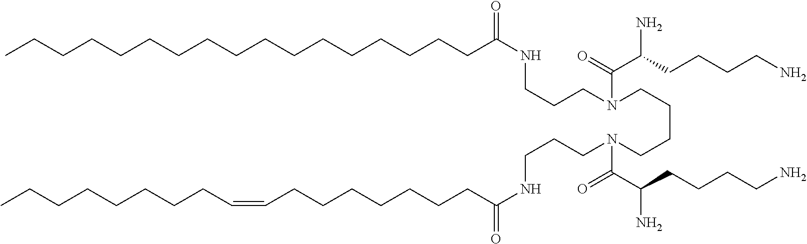

HRMS (ESI): t_{R}=11.79 min; m/z calcd (C₅₈H₁₁₇N₈O₄) 989.9198, found 989.9206 [M+H]⁺.

### Example 3

### GSC103 L-Orn, Oleic Acid, Stearic Acid

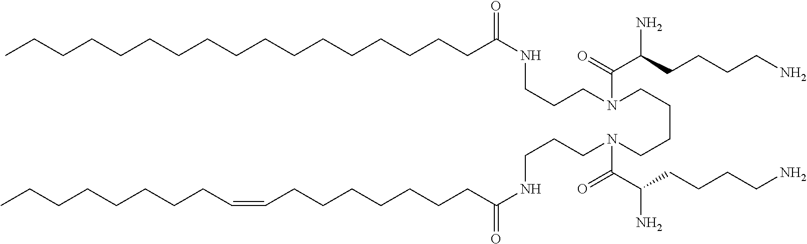

HRMS (ESI): t_{R}=11.80 min; m/z calcd (C₅₆H₁₁₃N₈O₄) 961.8885, found 961.8873 [M+H]⁺.

### 2) Pentamine-Based Gemini Compounds

### Description 9

### N¹,N⁸-Bis(trifluoroacetyl)-spermidine trifluoroacetate trifluoroacetic acid salt

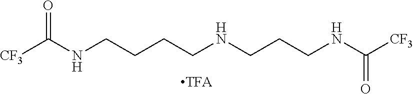

To a solution of spermidine (m=3, n=4; 8.0 g, 55.0 mmol) in CH₃CN (150 mL) and water (2.0 mL) was added ethyl trifluoroacetate (33.0 mL, 275 mmol) and the mixture was heated at reflux for 3 h. After cooling to room temperature, the solvent evaporated in vacuo. The residual solid was triturated with CH₂Cl₂ (2×150 mL) to afford the title trifluoroacetic acid salt as a white solid (21.0 g).

LC-MS (ESI): t_{R}=1.10 min (m/z=338.1 [M+H]⁺).

### Description 10

### N⁴-(tert-Butoxycarbonyl)-N¹,N⁸-bis(trifluoroacetyl)-spermidine

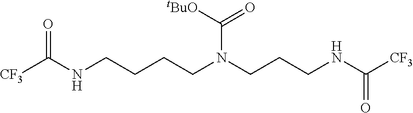

A solution of di-tent-butyl dicarbonate (11.3 g, 51.3 mmol) and triethylamine (75.0 mL, 54.0 mmol) in THF (25 mL) were added to N¹,N⁸-bis(trifluoroacetyl)spermidine trifluoroacetate of description 9 (21.0 g, 46.7 mmol) under a nitrogen atmosphere. After 18 h at rt., the solvent was evaporated in vacuo and EtOAc (500 mL) was added. The solution was washed successively with 5% aqueous NaHCO₃ (2×150 mL) and brine (150 mL), dried (Na₂SO₄), and evaporated in vacuo to leave the Boc carbamate as white solid (20.0 g).

LC-MS (ESI): t_{R}=4.09 min (m/z=438.3 [M+H]⁺).

### Description 11

### N⁴-(tert-Butoxycarbonyl)-spermidine

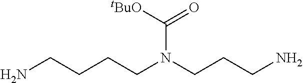

Aqueous sodium hydroxide solution (100 mL×0.5N) was added at 10° C. to a stirring solution of N⁴-(tert-butoxycarbonyl)-N¹,N⁸-bis(trifluoroacetyl)-spermidine of description 10 (20.0 g, 45.7 mmol) in MeOH (500 mL). The cooling bath was removed and the mixture was stirred for 18 h before the MeOH was evaporated in vacuo. The resulting aqueous suspension was extracted with [9:1] CHCl₃-MeOH (5×300 mL), and the combined organic extracts were dried (Na₂SO₄), and evaporated in vacuo to leave the Boc carbamate as a colourless oil (10.0 g).

LC-MS (ESI): t_{R}=2.15 min (m/z=246.2 [M+H]⁺).

### Description 12

### [4-(2-Cyano-ethylamino)-butyl]-[3-(2-cyano-ethylamino)-propyl]-carbamic acid tert-butyl ester

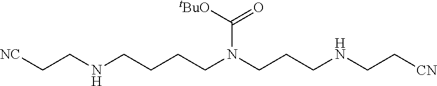

Acrylonitrile (2.15 mL, 32.6 mmol) was slowly added over 2 h to a stirring solution of the Boc carbamate of description 11 (4.0 g, 16.3 mmol) in MeOH (50 mL) maintained at 0° C. The resulting mixture was maintained at room temperature for a further 18 h and then concentrated in vacuo. The residue obtained was purified by column chromatography (silica gel) eluting with MeOH:EtOAc [10:90] to give the title bis-nitrile as a colourless viscous oil (5.00 g).

LC-MS (ESI): t_{R}=2.15 min (m/z=352.1 [M+H]⁺).

### Description 13

### {4-[tert-Butoxycarbonyl-(2-cyano-ethyl)-amino]-butyl}-{3-[tert-butoxycarbonyl-(2-cyano-ethyl)-amino]-propyl}-carbamic acid tert-butyl ester

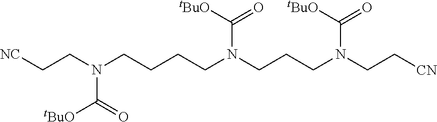

A solution of di-tert-butyl dicarbonate (3.40 g, 15.64 mmol) in THF (15 mL) was added to a solution of bis-nitrile of description 12 (2.5 g, 7.11 mmol) in a mixture of THF (10 mL) and triethylamine (15 mL) under a nitrogen atmosphere. After 18 h at room temperature, the solvent was evaporated in vacuo and EtOAc (100 mL) was added. The organic solution was washed successively with 5% aqueous NaHCO₃ solution (2×50 mL) and brine (50 mL), dried (Na₂SO₄), and evaporated in vacuo to afford the tris-Boc carbamate as pale yellow liquid (3.9 g).

¹H-NMR (CDCl₃): δ_{H} 1.45 (m, 31H), 1.75 (m, 2H), 2.60 (m, 4H), 3.16 (m, 4H), 3.26 (m, 4H), 3.45 (m, 4H).

### Description 14

### {4-[(3-Amino-propyl)-tert-butoxycarbonyl-amino]-butyl}-{3-[(3-amino-propyl)-tert-butoxycarbonyl-amino]-propyl}-carbamic acid tert-butyl ester

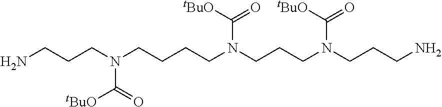

A mixture of tris-Boc nitrile of description 13 (3.90 g, 7.06 mmol), NaOH (0.45 g, 11.2 mmol) and Raney Nickel (2.1 g) in 95% ethyl alcohol (30 mL) was stirred at room temperature under a hydrogen atmosphere (1 atmos.) for 18 h. The catalyst was removed by filtration and the filtrate was concentrated in vacuo to 10 mL and treated with 40% aqueous NaOH solution (20 mL) and MeOH (10 mL). An oil separated which was extracted with CHCl₃ (2×100 mL). The combined organic extracts were dried (Na₂SO₄) and concentrated in vacuo to leave the title diamine as a pale yellow oil (3.90 g).

¹H-NMR (CDCl₃): δ_{H} 1.45 (brs, 31H), 1.63 (m, 4H), 1.73 (m, 2H), 2.67 (m, 4H), 3.20 (m, 12H).

### Description 15

### Octadec-9-enoic acid (3-amino-propyl)-(4-{3-[(3-amino-propyl)-octadec-9-enoyl-amino]-propylamino}-butyl)-amide tris-trifluoroacetic acid salt

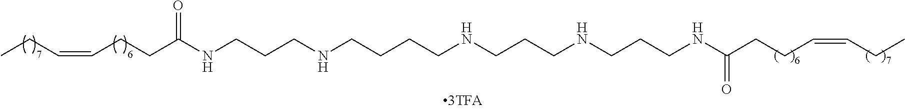

A solution of oleic acid N-hydroxysuccinimide ester (2.78 g, 7.32 mmol) in THF (50 mL) and a solution of potassium carbonate (1.08 g, 7.86 mmol) in water (10 mL) were added to a solution of description 14 (632 mg, 2.58 mmol) in THF (40 mL). The resulting mixture was stirred at room temperature for 18 h and then concentrated in vacuo. The residue was dissolved in ethyl acetate (300 mL), washed with water (150 mL×2) then dried (Na₂SO₄) and concentrated in vacuo to leave the tris-Boc carbamate as a colourless, viscous oil. The oil was dissolved in CH₂Cl₂ (25 mL) and treated with trifluoroacetic acid (15 mL). The resulting mixture was stirred at room temperature for 2 h, then concentrated in vacuo and the residue co-evaporated with diethyl ether (200 mL) to afford the title tris-trifluoroacetic salt as a white solid (3.72 g).

LC-MS (ESI): t_{R}=3.94 min (m/z=788.7 [M+H]⁺).

### Description 16

### General Procedure to Prepare N¹,N⁸-Dioleyl-N⁴-tris-(Aa)ₓ-pentamine Hydrochloride Salts

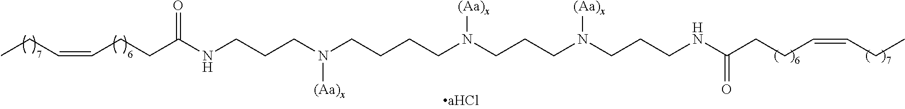

The N-terminal-protected amino acid ((PG)_{y}(Aa)ₓ: 3.5 mol eq.) TBTU (298 mg, 0.93 mmol), HOBt (125 mg, 0.93 mmol) and diisopropylethylamine (0.20 g 1.59 mmol) were added to a solution of tris-amine from description 15 (300 mg, 0.27 mmol) in CH₂Cl₂ (15 mL). After stirring at room temperature for 18 h, the reaction mixture was concentrated in vacuo and the residue was dissolved in EtOAc (10 mL). The organic solution was washed with water (2×10 mL), dried (Na₂SO₄), and concentrated in vacuo to leave an oil that was purified by column chromatography (silica gel) eluting with MeOH:CH₂Cl_{2 [}5:95] to afford the intermediate Boc carbamate as an oil. This carbamate was dissolved in diethyl ether (2 mL) and treated with a solution of HCl in dioxane (4M, 4 mL). After stirring at room temperature for 18 h, the resulting white precipitate was collected by filtration, washed with anhydrous diethyl ether and dried in vacuo to afford the pentamine hydrochloride salt as a white powder (11-77%).

### Example 4

### (Aa)ₓ=L-Lys

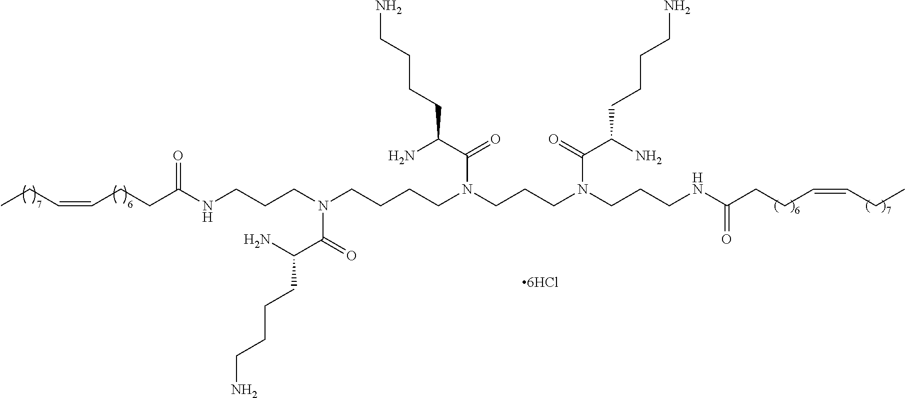

LC-MS (ESI): t_{R}=10.97 min (m/z=1173.1 [M+H]⁺); HRMS (ESI) m/z calcd (C₆₇H₁₃₄N₁₁O₅) 1173.0569, found 1173.0542 [M+H]⁺.

### Example 5

### (Aa)ₓ=D-Lys

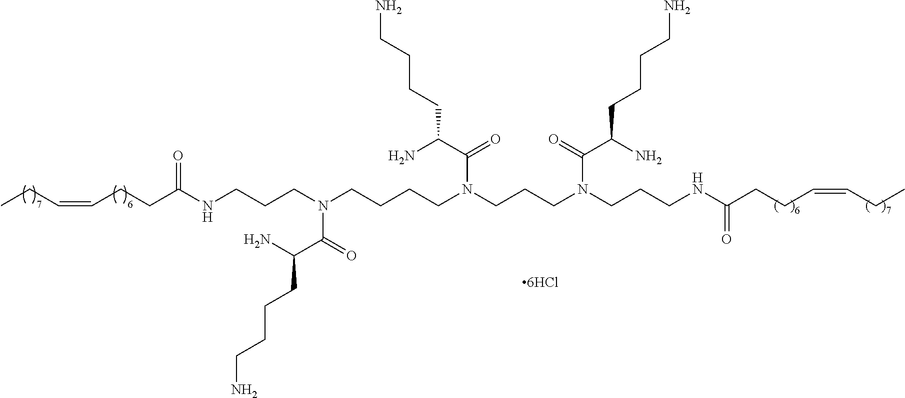

LC-MS (ESI): t_{R}=10.93 min (m/z=1173.1 [M+H]⁺); HRMS (ESI) m/z calcd (C₆₇H₁₃₄N₁₁O₅) 1173.0569, found 1173.0540 [M+H]⁺.

### Example 6

### (Aa)ₓ=L-Orn

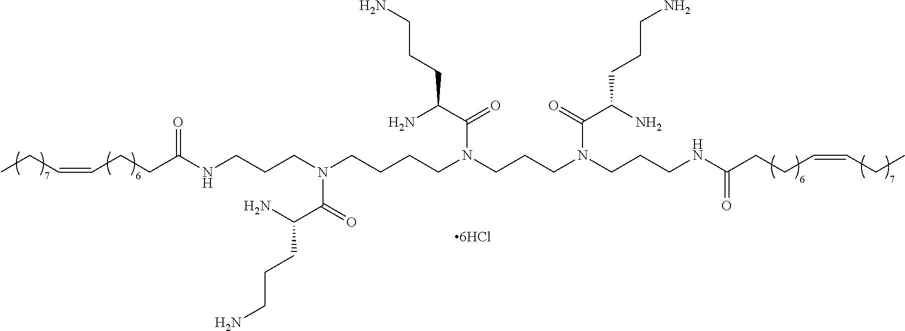

LC-MS (ESI): t_{R}=11.12 min (m/z=1131.0 [M+H]⁺); HRMS (ESI) m/z calcd (C₆₄H₁₂₈N₁₁O₅) 1131.0100, found 1131.0087 [M+H]⁺.

### Example 7

### (Aa)ₓ=L-Ser

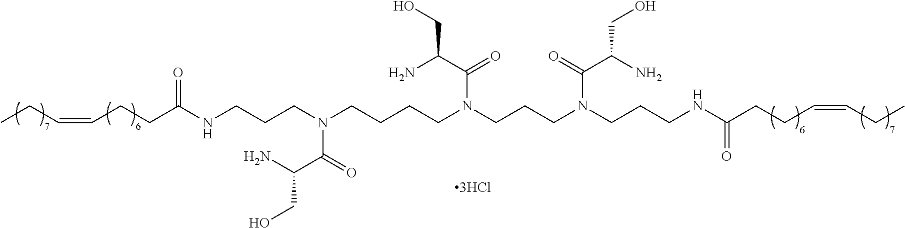

LC-MS (ESI): t_{R}=12.94 min (m/z=1049.9) [M+H]⁺); HRMS (ESI) m/z calcd (C₅₈H₁₁₃N₈O₈) 1049.8681, found 1049.8662 [M+H]⁺.

### Description 17

### [4-(3-Amino-propylamino)-butyl]-[3-(3-amino-propylamino)-propyl]-carbamic acid tert-butyl ester

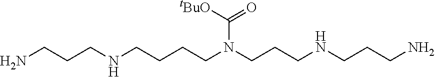

A mixture of the bis-nitrile of description 12 (3.10 g, 8.81 mmol), NaOH (0.3 g, 7.5 mmol) and Raney Nickel (1.5 g) in 95% ethyl alcohol (30 mL) was stirred at room temperature under a hydrogen atmosphere (1 atmos.) for 18 h. The catalyst was removed by filtration and the filtrate was concentrated in vacuo to 10 mL and treated with 40% aqueous NaOH solution (20 mL) and MeOH (10 mL). An oil separated which was extracted with CHCl₃ (2×100 mL). The combined organic extracts were dried (Na₂SO₄) and concentrated in vacuo to leave the amine title amine as a pale yellow oil (2.90 g).

¹H-NMR (MeOH): δ_{H} 1.42-1.61 (m, 13H), 1.62-1.80 (m, 6H), 2.52-2.75 (m, 12H), 3.18-3.33 (m, 4H).

### Description 18

### {4-[3-(2,2,2-Trifluoro-acetylamino)-propylamino]-butyl}-{3-[3-(2,2,2-trifluoro-acetylamino)-propylamino]-propyl}-carbamic acid tert-butyl ester

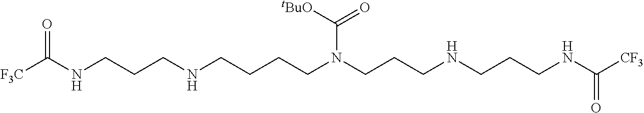

To a solution of the amine of description 17 (2.98 g, 8.23 mmol) in CH₃CN (100 mL) was added ethyl trifluoroacetate (5.88 mL, 49.39 mmol) and water (2.0 mL). The reaction mixture was heated at reflux for 3 h, then allowed to cool to room temperature and the solvent evaporated in vacuo. The residual solid was triturated first with CH₂Cl₂ (50 mL) and then with anhydrous diethyl ether (100 mL) to afford the bis-trifluoroacetic acid salt as a pale yellow solid (6.0 g).

¹H-NMR (DMSO): δ_{H} 11.35 (s, 9H), 1.45 (m, 4H), 1.78 (m, 6H), 2.88 (m, 8H), 3.07-3.19 (m, 4H), 3.25 (m, 4H), 8.48 (brs, 4H), 9.50 (m, 2H).

### Description 19

### {4-[(3-Amino-propyl)-octadec-9-enoyl-amino]-butyl}-{3-[(3-amino-propyl)-octadec-9-enoyl-amino]-propyl}-carbamic acid tert-butyl ester

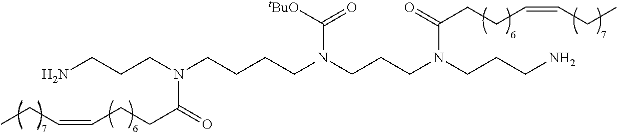

To a solution of oleic acid (1.60 g, 5.66 mmol) and the diamine of description 18 (2.00 g 2.57 mmol) in a mixture of CH₂Cl₂ (40 mL) and DMF (10 mL) were added TBTU (1.81 g, 5.66 mmol), HOBt (0.76 g, 5.66 mmol) and DIEA (1.99 g 15.42 mmol). After stirring at room temperature for 18 h, the reaction mixture was concentrated in vacuo. The residue was re-dissolved in CH₂Cl₂ (100 mL) and washed with 5% aqueous KHSO₄ (25 mL), 5% aqueous K₂CO₃ (2×25 mL) and brine (50 mL). The organic solution was dried (Na₂SO₄) and concentrated in vacuo to leave a gum that was purified by column chromatography (silica gel) eluting with a mixture of MeOH and CHCl_{3 [}3:97] to afford the intermediate trifluoroacetate as a colourless gum. The gum was dissolved in MeOH (10 mL) and water (2 mL) and K₂CO₃ (1.13 g, 8.12 mmol) were added. The resulting mixture was stirred at room temperature for 18 h, then concentrated in vacuo. The residue was dissolved in CH₂Cl₂ (100 mL) and washed successively with 5% aqueous K₂CO₃ (2×25 mL) and brine (50 mL), dried (Na₂SO₄), and evaporated in vacuo to afford the title diamine as a colourless gum (1.30 g).

LC-MS (ESI): t_{R}=4.51 min (m/z=888.8 [M+H]⁺).

### Description 20

### General Procedure to Prepare Protected N-hydroxysuccinimidyl Amino Acids (PG)_{y}(Aa)ₓOSuc

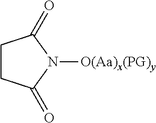

A solution of dicyclohexylcarbodiimide (1.05 eq.) in THF (15 mL) was added at room temperature with stirring to a mixture of N-hydroxysuccinimide (1.1 eq.) and the N-terminal protected amino acid (1 eq.) in anhydrous THF (10 mL). The mixture was stirred at room temperature for 18 h, then filtered to remove the precipitated solids. The residue was re-dissolved in CH₂Cl₂ (10 mL) and filtered twice more. Finally, the solvent was evaporated in vacuo to afford the N-hydroxysuccinimide ester as a white powder.

(Aa)ₓ(PG)_{y}=D-Lys(Boc)₂. LC-MS (ESI): t_{R}=3.88 min (m/z=345.1 [M−OSuc]⁺).
(Aa)ₓ(PG)_{y}=L-Orn(Boc)₂. LC-MS (ESI): t_{R}=3.79 min (m/z=331.1 [M−OSuc]⁺).
(Aa)ₓ(PG)_{y}=L-Ser(O^{t}Bu)(Boc). LC-MS (ESI): t_{R}=3.02 min (m/z=204.1 [M−OSuc]⁺).
(Aa)ₓ(PG)_{y}=L-Ser(O^{t}Bu)-L-Lys(Boc)₂. LC-MS (ESI): t_{R}=3.61 min (m/z=433.1 [M−OSuc]⁺).
(Aa)ₓ(PG)_{y}=[BocHN(CH₂)₃]₂NCH₂CO₂H. LC-MS (ESI): t_{R}=3.12 min (m/z=388.1 [M−OSuc]⁺).

### Description 21

### General Procedure to Prepare Bis-Oleyl Pentamine Hydrochloride Salts

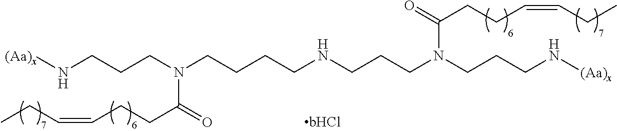

A solution of the protected N-hydroxysuccinimide amino acid ester (PG)_{y}(Aa)ₓOSuc (2.2 eq.) and the diamine (1.0 eq.) of description 19 in THF (30 mM) was treated at room temperature with a solution of K₂CO₃ in water (2.2 eq. 0.2M). The mixture was stirred for 18 h and then concentrated in vacuo. The residue was diluted with EtOAc (15 mM), washed with half the same volume of water, dried (Na₂SO₄) and the solvent evaporated in vacuo to leave a gum that was purified by column chromatography (silica gel) eluting with a mixture of MeOH and CHCl_{3 [}10:90] to afford the intermediate Boc carbamate as a gum. The gum was treated with a solution of HCl in diethyl ether (2M, 50 mM) at room temperature under nitrogen for 18 h when the precipitated solid was collected by filtration, washed with diethyl ether and dried in vacuo to afford the title bis-oleyl pentamine hydrochloride salt as a white powder (66-88% yield).

### Example 8

### (Aa)ₓ=L-Lys

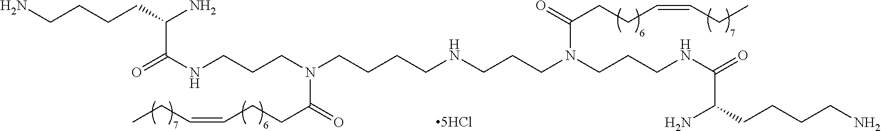

LC-MS (ESI): t_{R}=10.17 min (m/z=1044.96 [M+H]⁺); HRMS (ESI) m/z calcd (C₆₁H₁₂₂N₉O₄) 1044.9606, found 1044.9626 [M+H]⁺.

### Example 9

### (Aa)ₓ=D-Lys

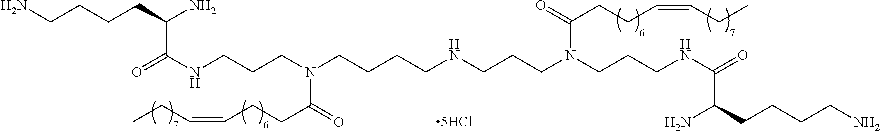

LC-MS (ESI): t_{R}=10.24 min (m/z=1044.96 [M+H]⁺); HRMS (ESI) m/z calcd (C₆₁H₁₂₂N₉O₄) 1044.9620, found 1044.9630 [M+H]⁺.

### Example 10

### (Aa)ₓ=L-Orn

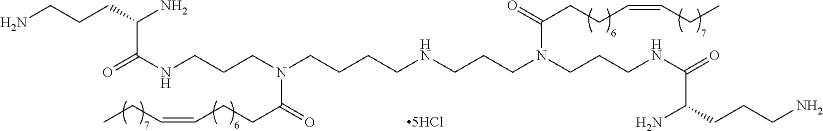

LC-MS (ESI): t_{R}=10.25 min (m/z=1016.93 [M+H]⁺); HRMS (ESI) m/z calcd (C₅₉H₁₁₈N₉O₄) 1016.9307, found 1016.9313 [M+H]⁺.

### Example 11

### (Aa)ₓ=L-Ser

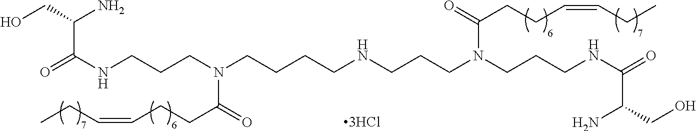

LC-MS (ESI): t_{R}=11.71 min (m/z=962.8364 [M+H]⁺); HRMS (ESI) m/z calcd (C₅₅H₁₀₈N₇O₆) 962.8361, found 962.8364 [M+H]⁺.

### Example 12

### (Aa)ₓ=L-Ser-L-Lys

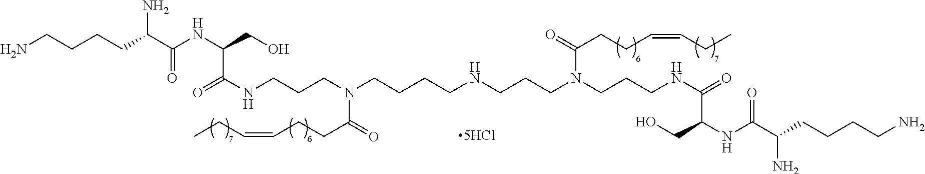

LC-MS (ESI): t_{R}=10.39 min (m/z=1219.03 [M+H]⁺), HRMS (ESI) m/z calcd (C₆₇H₁₃₂N₁₁O₈) 1219.0260, found 1219.0258 [M+H]⁺.

### Example 13

### (Aa)ₓ=[H₂N(CH₂)₃]₂NCH₂CO₂H

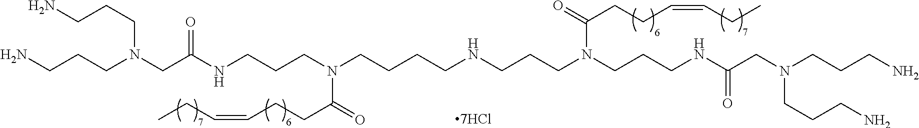

LC-MS (ESI): t_{R}=9.96 min (m/z=1131.05 [M+H]⁺); HRMS (ESI) m/z calcd (C₆₅H₁₃₂N₁₁O₄) 1131.0464, found 1131.0470 [M+H]⁺.

### Description 22

### 2,2,2-Trifluoro-N-[3-(4-{3-[3-(2,2,2-trifluoro-acetylamino)-propylamino]-propylamino}-butylamino)-propyl]-acetamide tris-trifluoroacetic acid salt

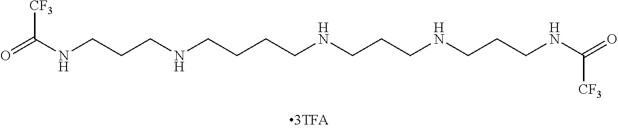

Trifluoroacetic acid (10 mL) was added at room temperature to a stirring solution of the Boc carbamate from description 18 (4.00 g, 5.14 mmol) in CH₂Cl₂ (10 mL). After 18 h, the mixture was concentrated in vacuo and the residue was treated with anhydrous diethyl ether (100 mL). The resulting precipitate was collected on a filter and washed with anhydrous diethyl ether (50 mL) to afford the title tris-trifluoroacetic acid salt as a white powder (4.00 g).

¹H-NMR (MeOH): δ_{H} 1.75 (m, 4H), 1.95 (m, 4H), 2.10 (m, 2H), 3.05 (m, 8H), 3.15 (m, 4H), 3.38 (m, 4H).

### Description 23

### Octadec-9-enoic acid {4-[(3-amino-propyl)-octadec-9-enoyl-amino]-butyl}-{3-[(3-amino-propyl)-octadec-9-enoyl-amino]-propyl}-amide

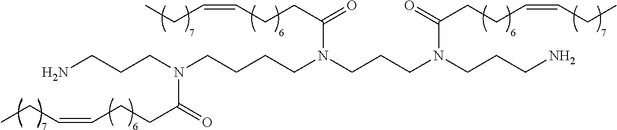

To a solution of oleic acid (3.00 g, 10.6 mmol), the compound of description 22 (2.50 g, 3.21 mmol) in CH₂Cl₂ (100 mL) were added TBTU (4.12 g, 12.8 mmol), HOBt (1.73 g, 12.8 mmol) and diisopropylethylamine (4.15 g 32.1 mmol). After stirring at room temperature for 18 h, the mixture was concentrated in vacuo and the residue was re-dissolved in CH₂Cl₂ (100 mL) and washed successively with 5% aqueous KHSO₄ (25 mL), 5% aqueous K₂CO₃ (2×25 mL), and brine (50 mL). The organic solution was dried (Na₂SO₄) and concentrated in vacuo to leave an oil which was purified by column chromatography (silica gel) eluting with a mixture of MeOH and CHCl_{3 [}3:97] to afford the trifluoroacetamide as a colourless gum.

The gum was dissolved in a mixture of MeOH (10 mL) and water (2 mL) and K₂CO₃ (1.13 g, 8.12 mmol) was added. This mixture was stirred at room temperature under nitrogen for 18 h and then concentrated in vacuo. The residue was diluted with CH₂Cl₂ (100 mL) and the organic solution was washed successively with 5% aqueous K₂CO₃ (2×25 mL) and brine (50 mL), dried (Na₂SO₄) and evaporated in vacuo to afford the title bis-amine as a colourless gum (1.50 g).

LC-MS (ESI): t_{R}=8.98 min (m/z=1053.4 [M+H]⁺).

### Description 24

### General Procedure to Prepare tris-oleyl,bis-(Aa)ₓ-pentamine Hydrochloride Salts

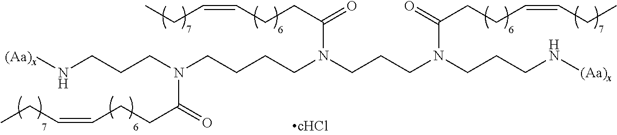

A solution of the protected N-hydroxysuccinimide amino acid ester (PG)_{y}(Aa)ₓOSuc (2.2 eq.) and the diamine (1.0 eq.) of description 23 in THF (20 mM) was treated at room temperature with a solution of K₂CO₃ in water (2.2 eq. 0.2M). The mixture was stirred for 18 h under nitrogen and then concentrated in vacuo. The residue was diluted with EtOAc (10 mM), washed with half the same volume of water, dried (Na₂SO₄) and the solvent evaporated in vacuo to leave a gum that was purified by column chromatography (silica gel) eluting with a mixture of MeOH and CHCl_{3 [}10:90] to afford the intermediate Boc carbamate as a gum. The gum was treated with a solution of HCl in diethyl ether (2M, 50 mM) at room temperature under nitrogen for 18 h and the precipitated solid was collected by filtration, washed with anhydrous diethyl ether and dried in vacuo to afford the title tris-oleyl pentamine hydrochloride salt as a white powder (41-56% yield).

### Example 14

### (Aa)ₓ=L-Lys

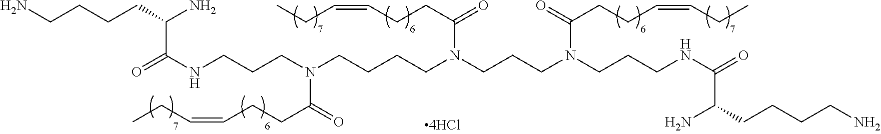

LC-MS (ESI): t_{R}=12.94 min (m/z=1309.20 [M+H]⁺); HRMS (ESI) m/z calcd (C₇₉H₁₅₄N₉O₅) 1309.2073, found 1309.2070 [M+H]⁺.

### Example 15

### (Aa)ₓ=D-Lys

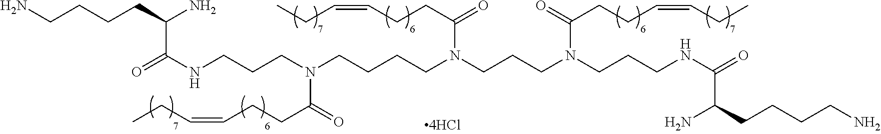

LC-MS (ESI): t_{R}=12.94 min (m/z=1309.20 [M+H]⁺); HRMS (ESI) m/z calcd (C₇₉H₁₅₄N₉O₅) 1309.2073, found 1309.2075 [M+H]⁺.

### Example 16

### (Aa)ₓ=L-Orn

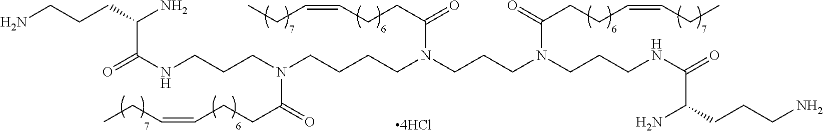

LC-MS (ESI): t_{R}=12.97 min (m/z=1281.17 [M+H]⁺); HRMS (ESI) m/z calcd (C₅₉H₁₁₈N₉O₅) 1281.1760, found 1281.1759 [M+H]⁺.

### Example 17

### (Aa)ₓ=L-Ser

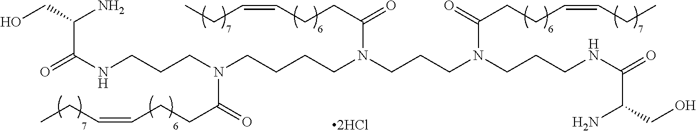

LC-MS (ESI): t_{R}=17.28 min (m/z=1227.08 [M+H]⁺); HRMS (ESI) m/z calcd (C₇₃H₁₄₀N₇O₇) 1227.0814, found 1227.0814 [M+H]⁺.

### Example 18

### (Aa)ₓ=L-Ser-L-Lys

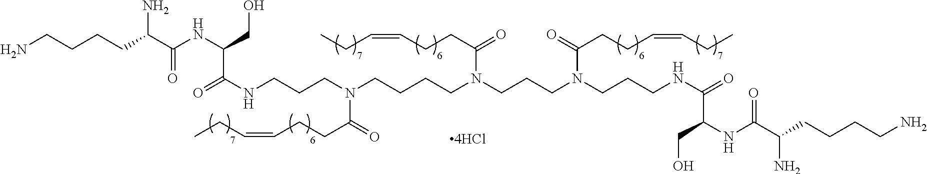

LC-MS (LC-TOF): t_{R}=3.17 min (1484.60 [M+H]⁺).

### Example 19

### (Aa)ₓ=[H₂N(CH₂)₃]₂NCH₂CO₂H

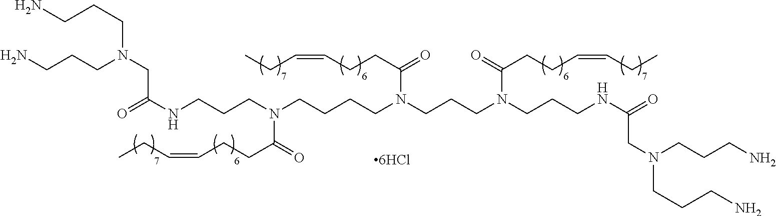

LC-MS (LC-TOF): t_{R}=3.83 min (m/z=1396.75 [M+H]⁺).

### Example 20

### Preparation of Bis-Oleyl Pentamine Hydrochloride Salt

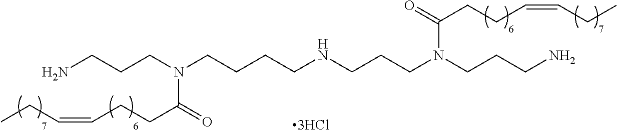

The mono-Boc diamine intermediate in description 19 (90.0 mg, 0.10 mmol) was treated with a solution of HCl in diethyl ether (2M, 5 mL) and stirred at room temperature under nitrogen for 3 h. The solvent was evaporated under a stream of nitrogen and the residual solid was washed with anhydrous diethyl ether (2 mL) and dried in vacuo to afford the title tris-hydrochloride salt as a white powder (85.0 mg).

LC-MS (ESI): t_{R}=12.28 min (m/z=788.77 [M+H]⁺); HRMS (ESI) m/z calcd (C₄₉H₉₈N₅O₂) 788.7721, found 788.7710 [M+H]⁺.

### Example 21

### Preparation of Tris-Oleyl Pentamine Hydrochloride Salt

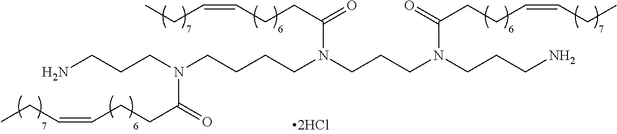

The tris-oleate of description 23 (85.0 mg, 81.0 μmol) was treated with a solution of HCl in diethyl ether (1.5M, 5 mL) and stirred at room temperature under nitrogen for 3 h. The solvent was evaporated under a stream of nitrogen and the residual white solid was washed with anhydrous diethyl ether (2 mL) and dried in vacuo to afford the title bis-hydrochloride salt as a white powder (70.0 mg).

LC-MS (ESI): t_{R}=17.63 min (m/z=1053.01 [M+H]⁺); HRMS (ESI) m/z calcd (C₆₇H₁₃₀N₅O₃) 1053.0174, found 1053.0181 [M+H]⁺.

### 3) Spermidine-Based Geminis

### Description 25

### N¹,N⁸-Bis(trifluoroacetyl)-spermidine trifluoroacetate

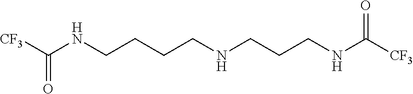

To a solution of spermidine (m=4, n=3; 9.70 g, 66.8 mmol) in CH₃CN (150 mL) was added ethyl trifluoroacetate (39.8 mL, 330 mmol) and water (2.8 mL, 18 mmol). The reaction mixture was heated at reflux for 18 h, then allowed to cool to room temperature and the solvent evaporated in vacuo. The residual solid was triturated with CH₂Cl₂ (2×150 mL) to give the title bis(trifluoroacetamide) as a white solid (28.9 g).

LC-MS (ESI): t_{R}=1.10 min (m/z=338.1 [M+H]⁺).

### Description 26

### N⁴-(tert-Butoxycarbonyl)-N¹,N⁸-bis(trifluoroacetyl)-spermidine

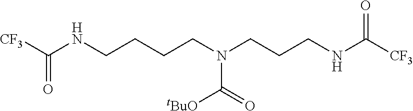

Diisopropylethylamine (16.7 mL, 95.7 mmol) and a solution of di-tert-butyl dicarbonate (14.63 g, 67.0 mmol) in THF (100 mL) were added to a solution of N¹,N⁸-bis(trifluoroacetyl)spermidine trifluoroacetate (description 25; 28.8 g, 63.8 mmol) in THF (280 mL) under a nitrogen atmosphere. After 18 h at rt., the solvent was evaporated in vacuo and EtOAc (700 mL) was added. The solution was washed successively with 5% NaHCO₃ (2×150 mL), brine (150 mL), 5% KHSO₄ (2×150 mL) and brine (2×150 mL), dried over Na₂SO₄, and evaporated to give the title Boc carbamate as white solid (28.0 g).

LC-MS (ESI): t_{R}=4.09 min (m/z=438.3 [M+H]⁺).

### Description 27

### N⁴-(tert-Butoxycarbonyl)-spermidine

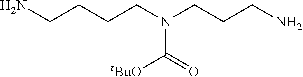

Aqueous sodium hydroxide solution (280 mL×0.5N) was added at 10° C. with stirring to a solution of N⁴-(tert-butoxycarbonyl)-N¹,N⁸-bis(trifluoroacetyl)-spermidine (description 26; 28.0 g, 64.0 mmol) in methanol (400 mL). The cooling bath was removed and the mixture was stirred for 18 h when the methanol was evaporated in vacuo. The resulting aqueous suspension was extracted with [9:1] CHCl₃-MeOH (5×300 mL) and the combined organic extracts were dried (Na₂SO₄) and evaporated in vacuo to leave the title amine as a colourless oil (15.5 g).

LC-MS (ESI): t_{R}=0.56 min (m/z=246.2 [M+H]⁺).

### Description 28

### N¹,N⁸-Dioleyl-spermidine trifluoroacetate

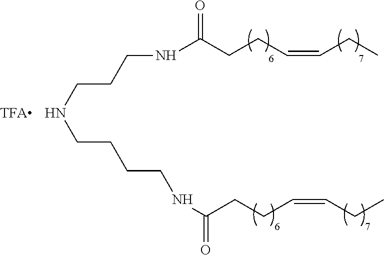

A solution of oleic acid N-hydroxysuccinimide ester (2.10 g, 5.40 mmol) dissolved in THF (90 mL) and a solution of potassium carbonate (890 mg, 6.40 mmol) in water (14 mL) were added to a solution of N⁴-(tert-butyloxycarbonyl)-spermidine (description 27; 632 mg, 2.58 mmol) in THF (90 mL). The resulting mixture was stirred at rt. for 16 h and then concentrated in vacuo. The residue was dissolved in CHCl₃ (300 mL), washed with 5% aqueous citric acid (50 mL) and brine (2×50 mL), then dried (Na₂SO₄) and concentrated in vacuo. The resulting oil was purified by flash chromatography eluting with hexane-EtOAc [75:25] followed by hexane-EtOAc [75:25] to afford the intermediate Boc carbamate as a brown oil. The oil was dissolved in CH₂Cl₂ (6.0 mL), cooled to 0° C. and treated with trifluoroacetic acid (6.0 mL). The resulting mixture was allowed to warm slowly to rt. and stirred for a further 1.5 h. The solvent was removed under reduced pressure, and the residue was co-evaporated with diethyl ether to afford the title trifluoroacetate salt as a white solid (1.94 g).

LC-MS (ESI): t_{R}=19.03 min (m/z=674.6564 [M+H]⁺, 100%); HRMS (ESI) m/z calcd (C₅₄H₁₀₇N₈O₄) 674.6564, found 674.6564 [M+H]⁺).

### Description 29

### 2,2,2-Trifluoro-N-{3-[3-(2,2,2-trifluoroacetylamino)-propylamino]propyl}-acetamide trifluoroacetate

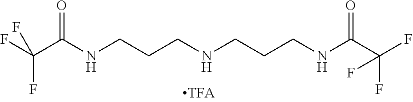

Ethyl trifluoroacetate (27.0 g, 190 mmol) was added to a solution of N¹-(3-aminopropyl)propane-1,3-diamine (m=n=3; 5.00 g, 38.2 mmol) in [99:1] acetonitrile:water (150 mL) and the mixture was heated at reflux with stirring for 3 h. After cooling to rt. CH₂Cl₂ (50 mL) was added and the resulting precipitate was collected and washed with CH₂Cl₂ (50 mL) to afford the title trifluoroacetate salt as a white powder (16.6 g).

LC-MS (ESI): t_{R}=2.05 min (m/z=323.0 [M+H]⁺).

### Description 30

### {Bis-[3-(2,2,2-trifluoroacetylamino)propyl]-amino}-acetic acid ethyl ester

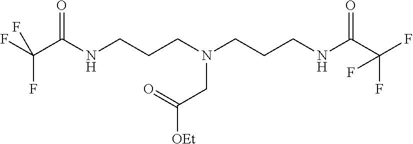

Ethyl bromoacetate (7.30 g, 43.7 mmol) was added to a solution of diisopropylethylamine (15.0 g, 86.3 mmol) and the trifluoroacetate salt (description 29; 15.0 g, 34.5 mmol) in anhydrous acetonitrile (150 mL). The resulting mixture was stirred at rt. for 18 h and then concentrated in vacuo. The residue obtained was diluted with water (100 mL) and extracted with CH₂Cl₂ (2×60 mL). The combined organic extracts were dried (Na₂SO₄) and evaporated in vacuo to give the title ethyl ester as a pale yellow oil (14.1 g).

LC-MS (ESI): t_{R}=3.0 min (m/z=410 [M+H]⁺).

### Description 31

### Bis-(3-tert-butoxycarbonylamino-propyl)-amino]-acetic acid

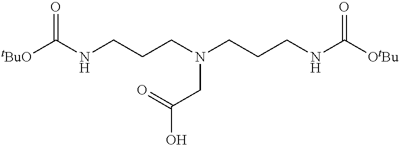

A mixture of the ethyl ester of description 30 (14.1 g, 34.5 mmol), sodium hydroxide (6.0 g, 40.0 mmol), water (35 mL) and ethanol (175 mL) was stirred at rt. for 20 h. Boc anhydride (15.1 g, 68.8 mmol) was added and stirring was continued for a further 3 h before the mixture was adjusted to neutral pH by adding 10% hydrochloric acid. Water (100 mL) was added and the solution was extracted with CH₂Cl₂ (4×50 mL). The combined organic extracts were dried (Na₂SO₄) and concentrated in vacuo to leave the title carboxylic acid as a white powder (3.5 g).

LC-MS (ESI): t_{R}=3.05 min (m/z=390.3 [M+H]⁺).

### Description 32

### Methyl Dicyanoacetate Potassium Salt

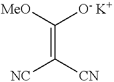

A solution of malonitrile (4.00 g, 60.6 mmol) and methyl chloroformate (4.95 mL, 64.2 mmol) in THF (9.0 mL) was added over 0.5 h to a stirring solution of potassium hydroxide (6.80 g, 121 mmol) in water (6.0 mL) maintaining the temperature below 40° C. The mixture was stirred at rt. for a further 2 h and the resulting precipitate was collected by filtration and washed first with cold water (10 mL) and then ethanol (10 mL) to afford the title potassium salt as a white solid (3.31 g).

¹H-NMR (D₂O): δ_{H} 3.58 (s, 3H).

### Description 33

### Methyl 3-amino-2-(aminomethyl)propionate bis-hydrochloride

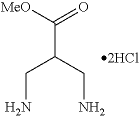

A stirring suspension of 10% palladium on carbon (2.0 g) in a solution of the sodium salt of methyl dicyanoacetate (1.00 g, 6.20 mmol) in methanol (100 mL) containing conc^{d} hydrochloric acid (32%, 2.0 mL) was hydrogenated at 5 bar and rt. for 24 h. The catalyst was removed by filtration and the solvent was evaporated in vacuo. The residue was treated with methanol (50 mL) and the precipitated sodium chloride was removed by filtration. The solution was concentrated to a low volume in vacuo and then EtOAc was added to precipitate the title bis-hydrochloride as an off-white solid (1.20 g).

¹H-NMR (d⁶ DMSO): δ_{H} 8.40 (brs, 6H), 3.65 (s, 3H), 3.25 (m, 1H), 3.15 (m, 4H).

### Description 34

### 3-(tert-Butoxycarbonylamino)-2-(tert-butoxycarbonylamino-methyl)propionic acid methyl ester

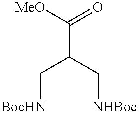

Boc anhydride (0.62 g, 2.83 mmol) was added to a stirring solution of diisopropylethylamine (0.95 mL, 5.40 mmol) and the bis-hydrochloride of description 33 (255 mg, 1.35 mmol) in DMF (8.0 mL). After stirring for 18 h at rt. the precipitated solids were removed by filtration and the filtrate was evaporated in vacuo. The residual oil was dissolved in EtOAc (30 mL) then washed with water (2×5 mL), dried (Na₂SO₄) and evaporated in vacuo to afford the title bis-Boc carbamate as a viscous colourless oil (430 mg).

¹H-NMR (CDCl₃): δ_{H} 5.20 (brs, 2H), 3.68 (s, 3H), 3.55 (m, 2H), 3.20 (m, 2H), 2.73 (m, 1H), 1.42 (s, 18H).

### Description 35

### 3-(tert-Butoxycarbonylamino)-2-(tert-butoxycarbonylamino-methyl)propionic acid

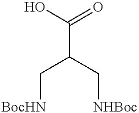

Aqueous 2N sodium hydroxide (2.48 mL, 4.96 mmol) was added to a stirring solution of the methyl ester of description 34 (0.41 g, 1.24 mmol) in THF (7.5 mL) and the mixture was stirred at rt. for 24 h. Water (15 mL) was added and the solution was acidified with 5% aqueous citric acid. The mixture was extracted with CHCl₃ (3×25 mL) and the combined organic extracts were washed with brine (30 mL), dried (Na₂SO₄), and evaporated in vacuo to leave the title acid as a viscous, colourless oil (280 mg).

¹H-NMR (d₆ DMSO): δ_{H} 12.2 (brs, 1H), 6.70 (brs, 2H), 3.05 (m, 4H), 2.50 (m, 1H), 1.35 (s, 18H).

### Description 36

### (Boc)₂Lys.(^{t}BuO)SerOH

A solution of bis-Boc L-lysine N-hydroxysuccinimide (2.54 g, 5.72 mmol) in THF (60.0 mL) was added to a stirring mixture of L-serine tert-butyl ether (1.02 g, 6.30 mmol) and potassium carbonate (1.03 g, 7.44 mmol) in water (12.0 mL). The mixture was stirred at room temperature for 18 h and then concentrated in vacuo. The residue was diluted with CHCl₃ (140 mL) and water (140 mL) and adjusted to pH2 with 1N hydrochloric acid. The organic layer was separated and the aqueous phase was extracted with CHCl₃ (2×140 mL). The combined organic phases were dried (Na₂SO₄) and then evaporated in vacuo to afford the dipeptide as a white foam (2.84 g).

LC-MS (ESI): t_{R}=3.80 min (m/z=490.3 [M+H]⁺).

### Description 37

### General Procedure to Prepare N¹,N⁸-dioleyl-N⁴-(Aa)-spermidine

The N-terminal-protected amino acid ((Aa)ₓ-(PG)_{y}: 1.1 eq.), HCTU (1.1 eq.), and diisopropylethylamine (3.2 eq.) were added to a solution of N¹,N⁸-dioleyl-spermidine trifluoroacetate (description 28) in DMF (60 mM). The mixture was stirred at rt. under N₂ for 18 h when an equal volume of EtOAc was added. The organic mixture was washed successively with 5% aqueous KHSO₄ solution (3×), 5% aqueous K₂CO₃ solution (3×) and brine (1×), then dried (Na₂SO₄) and concentrated in vacuo. The residue was dissolved in EtOAc (10 mM) and an equal volume of 5.0N HCl in EtOAc was added. The reaction was stirred at rt. for 2 h and then concentrated in vacuo and the residue triturated with diethyl ether to afford an amorphous white solid which was purified by preparative MS-directed RP-HPLC (C-18, 5 μm; eluent A: Water+0.1% formic acid, eluent B: MeCN:Water [95:5]+0.1% formic acid; flow rate: 40 mL/min; detector (ESI-MS). method: 5-30% B in A over 15 min). The fraction containing the product was evaporated and the residue was dissolved in methanol and treated with 2.0N HCl in diethyl ether was added. After 10 min, the solvent was removed in vacuo and the residue was lyophilized to afford the title surfactant hydrochloride as a white solid.

### Example 22

### Aa=L-Dap

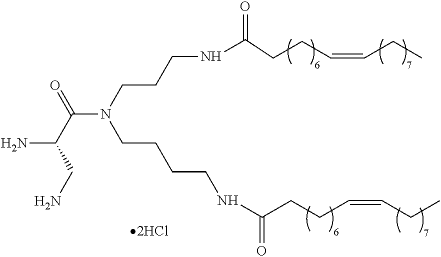

LC-MS (ESI): t_{R}=7.50 min (m/z=674.6 [M−Dap]⁺ (100%), 760.7 [M+H]⁺ (20%)); HRMS (ESI) m/z calcd (C₄₆H₉₀N₅O₃) 760.7044, found 760.7028 [M+H]⁺.

### Example 23

### Aa=L-Dab

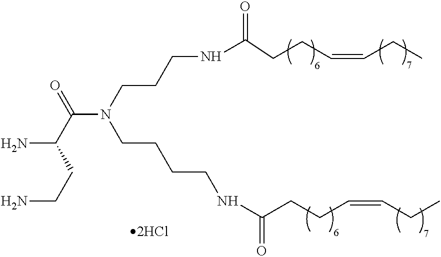

LC-MS (ESI): t_{R}=6.70 min (m/z=774.7 [M+H]⁺ (50%)); HRMS (ESI) m/z calcd (C₄₇H₉₂N₅O₃) 774.7200, found 774.7197 [M+H]⁺.

### Example 24

### Aa=L-Orn

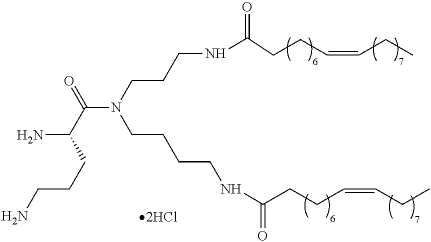

LC-MS (ESI): t_{R}=6.64 min (m/z=788.7 [M+H]⁺ (100%)); HRMS (ESI) m/z calcd (C₄₈H₉₄N₅O₃) 788.7357, found 788.7350 [M+H]⁺.

### Example 25

### Aa=L-Lys

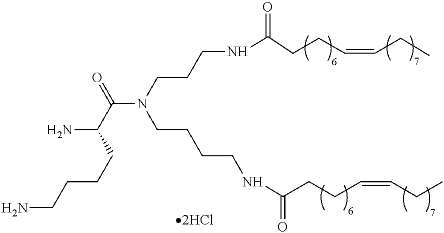

LC-MS (ESI): t_{R}=6.68 min (m/z=802.8 [M+H]⁺ (100%)); HRMS (ESI) m/z calcd (C₄₉H₉₆N₅O₃) 802.7513, found 802.7517 [M+H]⁺.

### Example 26

### Aa=D-Lys

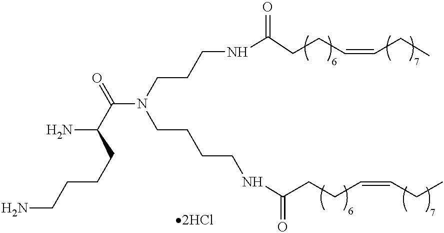

LC-MS (ESI): t_{R}=6.78 min (m/z=802.8 [M+H]⁺ (100%)); HRMS (ESI) m/z calcd (C₄₉H₉₆N₅O₃) 802.7513, found 802.7524 [M+H]⁺.

### Example 27

### Aa=L-Ser

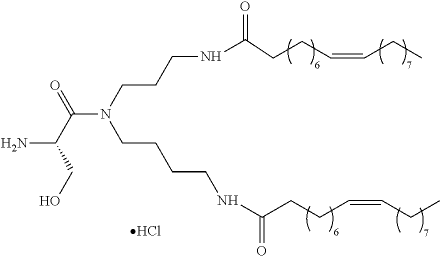

LC-MS (ESI): t_{R}=18.41 min (m/z=761.7 [M+H]⁺ (100%)); HRMS (ESI) m/z calcd (C₄₆H₈₉N₄O₄) 761.6884, found 761.6888 [M+H]⁺.

### Example 28

### Aa=L-Ser-L-Lys

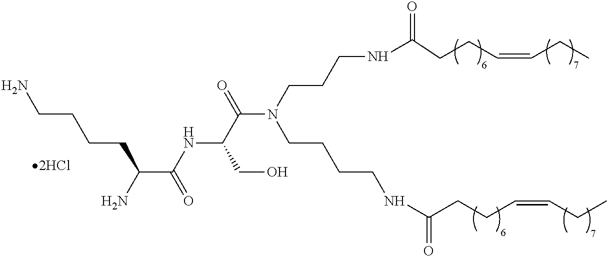

LC-MS (ESI): t_{R}=14.89 min (m/z=889.8 [M+H]⁺ (100%)); HRMS (ESI) m/z calcd (C₅₂H₁₀₁N₆O₅) 889.7833, found 889.7829 [M+H]⁺.

### Example 29

### Aa=L-Ser-D-Lys

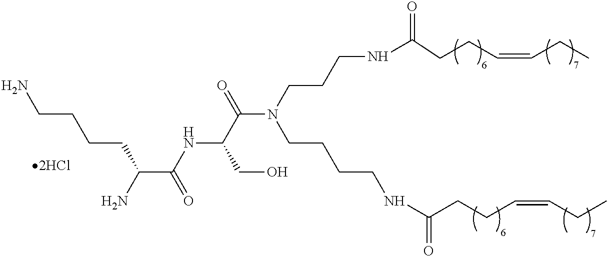

LC-MS (ESI): t_{R}=14.94 min (m/z=889.8 [M+H]⁺ (100%)); HRMS (ESI) m/z calcd (C₅₂H₁₀₁N₆O₅) 889.7833, found 889.7842 [M+H]⁺.

### Example 30

### Aa=(Description 35)

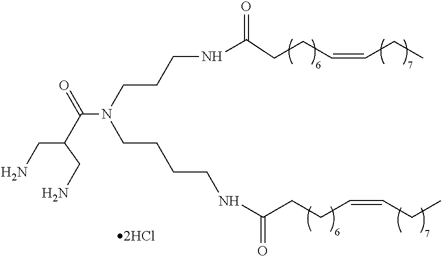

LC-MS (ESI): t_{R}=6.33 min (m/z=774.7 [M+H]⁺ (100%)).

### Example 31

### Aa=(Description 31)

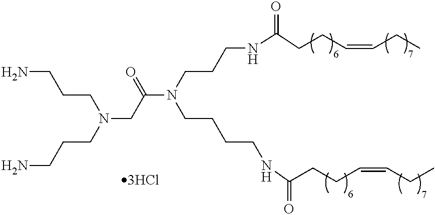

A solution of the bis-Boc carboxylic acid of description 31 (660 mg, 1.68 mmol) in CH₂Cl₂ (15 mL) containing TBTU (530 mg, 1.68 mmol) and diisopropylethylamine (1.5 mL 8.40 mmol) was added to N¹,N⁸-dioleyl-spermidine bis(trifluoroacetate) (description 28; 1.00 g, 1.40 mmol). The mixture was stirred at rt. For 18 h, then concentrated in vacuo and the residue taken up in CH₂Cl₂ (100 mL) and washed successively with 5% aqueous KHSO₄ solution (25 mL), 5% aqueous K₂CO₃ solution (2×25 mL) and brine (50 mL). The organic phase was dried (Na₂SO₄) and evaporated in vacuo to leave an oil which was purified by column chromatography (silica gel) eluting with a mixture of CHCl₃-MeOH [90:10] to afford the intermediate bis-Boc carbamate as a colourless gum. The gum was dissolved in CH₂Cl₂ (25 mL) and treated with 5N HCl in EtOAc (10 mL). The resulting mixture was stirred at rt. for 2 h when the precipitated solid was collected by filtration, washed with anhydrous diethyl ether and dried in vacuo to afford the title compound as a white powder.

LC-MS (ESI): t_{R}=12.65 min (m/z=845.8 [M+H]⁺ (100%)); HRMS (ESI) m/z calcd (C₅₁H₁₀₁N₆O₃) 845.7935, found 845.7938 [M+H]⁺.

### Description 38

### General Procedure to Prepare N¹,N⁸-dioleyl-N⁴-[(description 35)-Aa]-spermidine

The N-terminal-protected amino acid (2.6 eq.), HCTU (2.6 eq.), and DIEA (7.5 eq.) were added to a solution of example 30 in DMF (80 mM). The mixture was stirred at rt. under N₂ for 18 h and then an equal volume of EtOAc was added. The organic mixture was washed successively with 5% aqueous KHSO₄ solution (3×), 5% aqueous K₂CO₃ solution (3×) and brine (1×), then dried (Na₂SO₄) and concentrated in vacuo. The residue was dissolved in EtOAc (10 mM) and an equal volume of 5.0N HCl in EtOAc was added. The reaction was stirred at rt. for 2 h and then concentrated in vacuo and the residue triturated with diethyl ether to afford an amorphous white solid which was purified by preparative MS-directed RP-HPLC(C-18, 5 μm; eluent A: Water+0.1% formic acid, eluent B: MeCN:Water [95:5]+0.1% formic acid; flow rate: 40 mL/min; detector (ESI-MS). method: 30-50% B in A over 15 min). The fraction containing the product was evaporated and the residue was dissolved in methanol and treated with 2.0M HCl in diethyl ether. After 10 min, the solvent was removed in vacuo and the residue was lyophilized to afford the title hydrochloride salt as a white solid.

### Example 32

### Aa=L-Dab

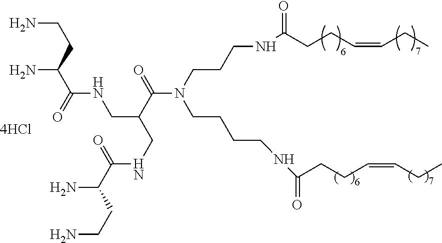

LC-MS (ESI): t_{R}=12.65 min (m/z=974.8 [M+H]⁺ (100%)); HRMS (ESI) m/z calcd (C₅₅H₁₀₈N₉O₅) 974.8473, found 974.8473 [M+H]⁺.

### Example 33

### Aa=L-Lys

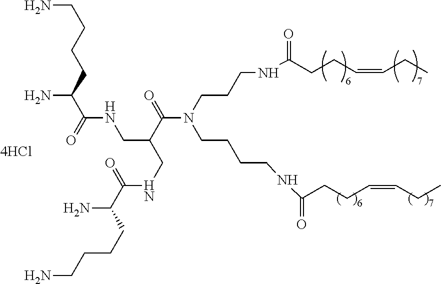

LC-MS (ESI): t_{R}=12.47 min (m/z=1030.9 [M+H]⁺ (100%)); HRMS (ESI) m/z calcd (C₅₉H₁₁₆N₉O₅) 1030.9099, found 1030.9108 [M+H]⁺.

### Example 34

### Aa=L-Ser-L-Lys

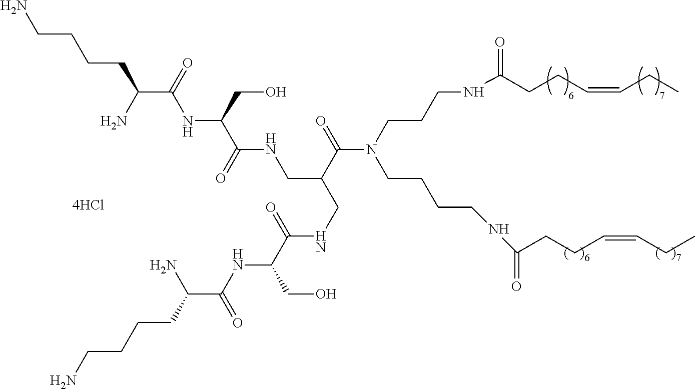

LC-MS (ESI): t_{R}=12.46 min (m/z=603.0 [M+2H]²⁺ (100%), 1205.0 [M+H]⁺ (85%)); HRMS (ESI) m/z calcd (C₆₅H₁₂₆N₁₁O₉) 1204.9740, found 1204.9755 [M+H]⁺.

### Description 39

### General Procedure to Prepare N¹,N⁸-dioleyl-N⁴-[(description 31)-Aa]-spermidine

The amine hydrochloride of example 31 (1.0 eq.) was added at rt. to a stirring solution of the N-terminal-protected amino acid ((Aa)ₓ(PG)_{y}; 2.2 eq.), 1-(3-dimethylaminopropyl)-3-ethylcarbodiimide hydrochloride (2.2 eq.) and diisopropylethylamine (6.0 eq.) in CH₂Cl₂ (approx. 20 mM). After 18 h, the reaction mixture was concentrated in vacuo, and the residue was dissolved in CH₂Cl₂ and washed successively with water, 5% aqueous K₂CO₃ solution, and brine. The organic phase was dried (Na₂SO₄) and concentrated in vacuo to leave an oil which was purified by column chromatography (silica gel) eluting with a mixture of MeOH:CHCl₃ [97:3] containing 0.2% triethylamine to afford the intermediate carbamate as a colourless gum. The gum was dissolved in 5N HCl in EtOAc (5 mM) and the resulting solution stirred at rt. for 2 h. The precipitated solid was collected by filtration, washed with anhydrous diethyl ether and dried in vacuo to afford the title hydrochloride salt as a white powder.

### Example 35

### Aa=L-Dap

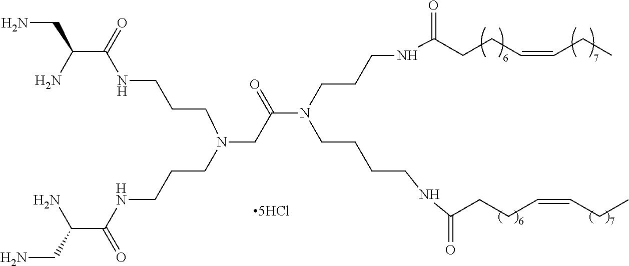

LC-MS (ESI): t_{R}=12.65 min (m/z=1017.9 [M+H]⁺ (100%)); HRMS (ESI) m/z calcd (C₆₇H₁₁₃N₁₀O₅) 1017.8895, found 1017.8915 [M+H]⁺.

### Example 36

### Aa=L-Dab

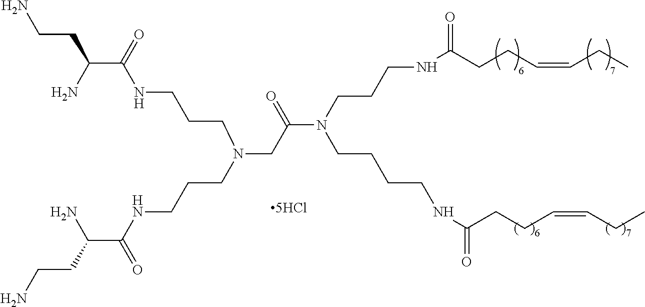

LC-MS (ESI): t_{R}=12.04 min (m/z=1045.9 [M+H]⁺ (100%)); HRMS (ESI) m/z calcd (C₆₉H₁₁₇N₁₀O₅) 1045.9208, found 1045.9229 [M+H]⁺.

### Example 37

### Aa=L-Lys

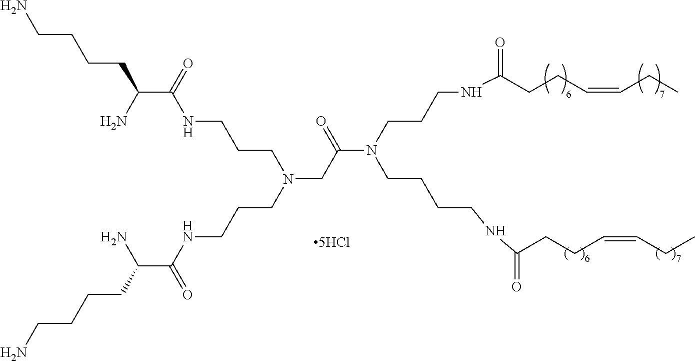

LC-MS (ESI): t_{R}=11.81 min (m/z=1101.9 [M+H]⁺ (80%)); HRMS (ESI) m/z calcd (C₆₃H₁₂₅N₁₀O₅) 1101.9834, found 1101.9818 [M+H]⁺.

### Example 38

### Aa=D-Lys

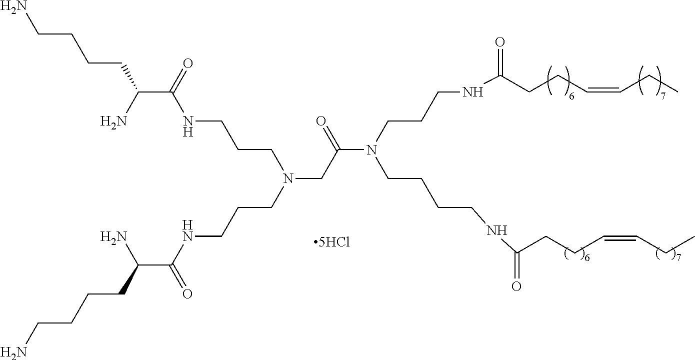

LC-MS (ESI): t_{R}=11.94 min (m/z=1101.9 [M+H]⁺ (80%)); HRMS (ESI) m/z calcd (C₆₃H₁₂₅N₁₀O₅) 1101.9834, found 1101.9835 [M+H]⁺.

### Example 39

### Aa=L-Ser

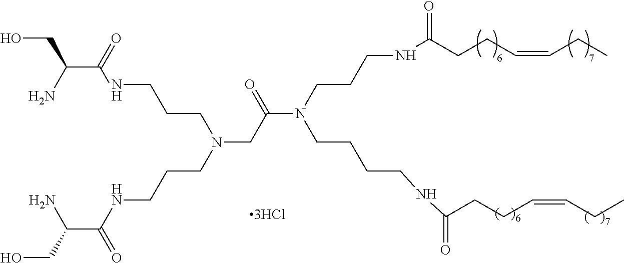

LC-MS (ESI): t_{R}=13.25 min (m/z=1019.8 [M+H]⁺ (100%)); HRMS (ESI) m/z calcd (C₅₇H111N₈O₇) 1019.8576, found 1019.8560 [M+H]⁺.

### Example 40

### Aa=L-Ser-L-Lys

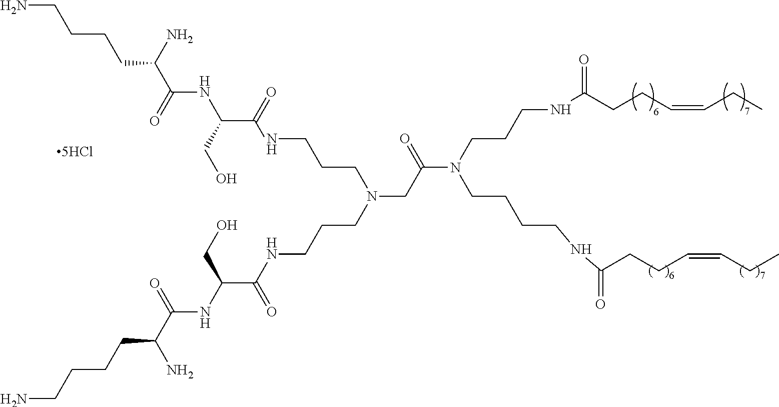

LC-MS (ESI): t_{R}=11.91 min (m/z=1276.0 [M+H]⁺ (100%), HRMS (ESI) m/z calcd (C₆₉H₁₃₅N₁₂O₉) 1276.0475, found 1276.0447 [M+H]⁺.

### Example 41

### Aa=(Description 31)

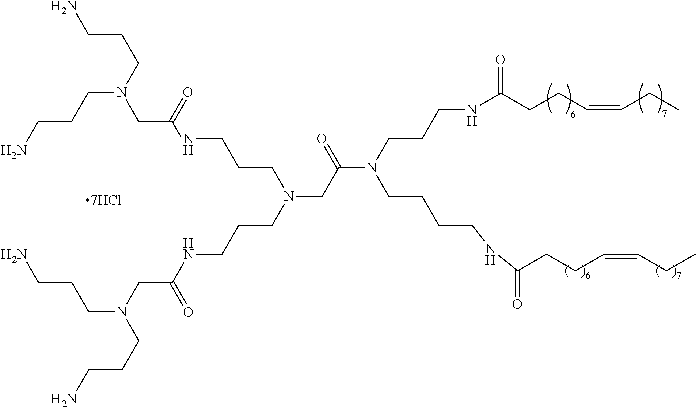

LC-MS (ESI): t_{R}=11.66 min (m/z=1188.0 [M+H]⁺ (100%)); HRMS (ESI) m/z calcd (C₆₇H₁₃₅N₁₂O₅) 1188.0678, found 1188.0686 [M+H]⁺.

### 4) Ester-Linked Geminis

### Description 40

### N,N′-Bis-(2-cyanoethyl)-1,4-diaminobutane

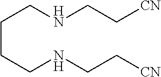

To a solution of 1,4-diaminobutane (n=4; 25.0 g, 0.28 mol) in methanol (50 mL) was added dropwise a solution of acrylonitrile (31.6 g, 0.60 mmol) in methanol (25 mL) at 0° C. After the addition the mixture was allowed to come to room temperature and then stirred for 18 h. Finally, the solvent was removed in vacuo, to give the title diamine as a pale yellow liquid, (57.0 g, quant.).

Rf_{Silica}: 0.60 (MeOH-0.88NH₃ 95:5).

¹H-NMR (CDCl₃): δ_{H} 2.88 (m, 4H), 2.60 (m, 4H), 2.46 (m, 4H), 1.45 (m, 4H).

### Description 41

### N,N′-Bis-(2-carbethoxyethyl)-1,4-diaminobutane dihydrochloride

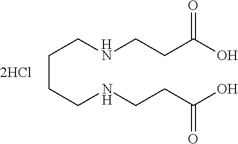

A solution of description 40 (n=4; 10.0 g, 51.5 mmol) in 6N HCl (80 mL) was heated at reflux for 18 h, then allowed to cool to room temperature and the solvent partially evaporated in vacuo. To the residual solution was added EtOH (40 mL) and the precipitated solid was filtered off and washed with EtOH (10 mL) to afford the title di-carboxylic acid as a white solid (17.4 g, quant.).

¹H-NMR (d⁶ DMSO): δ_{H} 12.70 (brs, 2H), 3.05 (t, J=7.5, 4H), 2.88 (m, 4H), 2.72 (t, 4H), 1.65 (m, 4H).

### Description 42

### N,N′-Bis-(2-carbethoxyethyl)-N,N′-bis-(tert-butoxycarbonyl)-1,4-diaminobutane

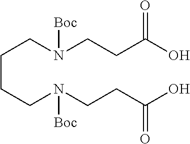

To a solution of description 41 (n=4; 19.9 g, 65.3 mmol) in 1N NaOH (350 mL) was added a solution of di-tert-butyl dicarbonate (57.0 g, 261 mmol) in dioxane (350 mL). The mixture was stirred at rt for 18 h, and then concentrated to half volume The residue was adjusted to pH 3-4, and then extracted with dichloromethane (250 mL×3). The combined organic extracts were washed with brine (150 mL), dried (Na₂SO₄) and concentrated in vacuo to leave the title carbamate as a white powder (24.4 g, 87%).

Rf_{Silica}: 0.31 (EtOAc-MeOH 2:1).

LC-MS (ESI): t_{R}=4.04 min (m/z=433.1 [M+H⁺]).

### Description 43

### 3-(tert-Butoxycarbonyl-{4-[tert-butoxycarbonyl-(2-octadec-8-enyloxycarbonyl-ethyl)-amino]-butyl}-amino)-propionic acid octadec-8-enyl ester

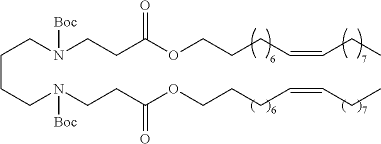

To a solution of the N-protected amino acid of description 42 (n=4; 15.2 g, 35.1 mmol), dimethylaminopyridine (1.70 g, 14.0 mmol) and oleyl alcohol (18.4 g, 68.4 mmol) in dichloromethane (200 mL) was added a solution of EDCl (13.1 g, 68.4 mmol), in dichloromethane (50 mL) at 0° C. The resulting mixture was allowed to warm to room temperature and then stirred under N₂ for 18 h. Dichloromethane (250 mL) was added and the mixture was washed with brine (4×200 mL), dried (Na₂SO₄) and concentrated in vacuo to leave an oil which was purified by column chromatography eluting with a solvent gradient of CH₂Cl₂ (50-90%) in hexane to afford the title ester as a colourless oil (13.0 g, 40%).

Rf_{Silica}: 0.61 (Hex-EtOAc 7:3).

¹H-NMR (CDCl₃): δ_{H} 5.32 (m, 4H), 4.05 (t, J=7.0, 4H), 3.42 (brs, 4H), 3.18 (brs, 4H), 2.53 (brs, 4H), 1.98 (m, 8H), 1.60 (m, 6H), 1.42 (m, 22H), 1.40-1.20 (m, 40H), 0.86 (t, J=7.0, 6H).

### Description 44

### 3-[4-(2-Octadec-9-enyloxycarbonyl-ethylamino)-butylamino]-propionic acid octadec-9-enyl ester bis hydrochloride salt

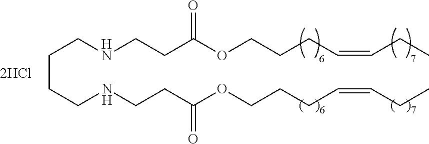

The ester of description 43 (n=4, R=oleyl; 13.0 g, 13.9 mmol) was dissolved in CH₂Cl₂ (70 mL) and treated with 4M HCl in EtOAc (140 mL). The resulting mixture was stirred at rt. for 2 h, then the solvent was removed in vacuo and the residue was triturated with anhydrous diethyl ether (150 mL) to afford a solid which was dried in vacuo to afford bis hydrochloride as a white powder (10.23 g, 91%).

Rf_{Silica}: 0.46 (MeOH-0.88NH₃ 97:3).

LC-MS (ESI): t_{R}=8.07 min (m/z=733.6 [M+H⁺]).

### Description 45

### General Procedure to Prepare Gemini Surfactants of Examples 42-45

The N-terminal-protected amino acid (0.41 mmol, 2.6 eq.), HCTU (168 mg, 0.41 mmol, 2.6 eq.), and diisopropylethylamine (0.19 mL, 1.10 mmol, 7.0 eq.) were added to a solution of the amine hydrochloride of description 44 (n=4, R=oleyl; 150 mg, 0.156 mmol) in DMF-CH₂Cl_{2 [}1:1] (4.0 mL). The mixture was stirred at rt. under N₂ for 18 h and then the mixture was concentrated to low volume and EtOAc (30 mL) was added. The organic solution was washed successively with 5% aqueous KHSO₄ solution (3×8 mL), 5% aqueous K₂CO₃ solution (3×8 mL) and brine (3×10 mL), then dried (Na₂SO₄) and concentrated in vacuo. The residue was purified by reverse phase column chromatography eluting with a solvent gradient of MeOH (50-100%) in water and then by silica gel column chromatography eluting with a solvent gradient of EtOAc (20-40%) in hexane. The residue was dissolved in EtOAc (2.0 mL) and 5.0N HCl in EtOAc (3.0 mL) was added. The resulting mixture was stirred at rt. for 2 h and then concentrated in vacuo and the solid residue triturated with diethyl ether (5.0 mL) to afford the gemini surfactants of examples 42 to 45 as white powders (75-93%).

### Example 42

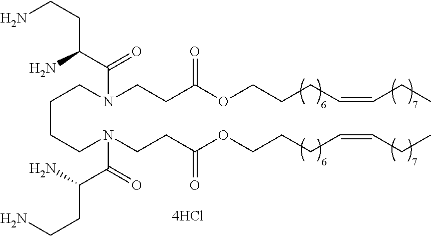

LC-MS (ESI): t_{R}=12.18 min (m/z=933.8 [M+H]⁺ (100%)); HRMS (ESI) m/z calcd (C₅₄H₁₀₅N₆O₆) 933.8096, found 933.8096 [M+H]⁺.

### Example 43

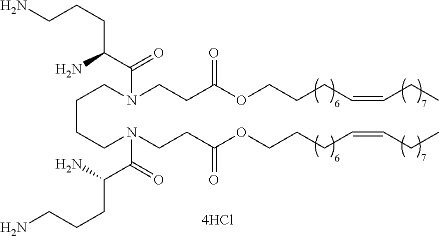

LC-MS (ESI): t_{R}=12.15 min (m/z=961.8 [M+H]⁺ (100%)); HRMS (ESI) m/z calcd (C₅₆H₁₀₉N₆O₆) 961.8409, found 933.8400 [M+H]⁺.

### Example 44

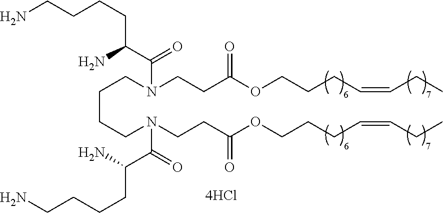

LC-MS (ESI): t_{R}=12.18 min (m/z=989.9 [M+H]⁺ (100%)); HRMS (ESI) m/z calcd (C₅₈H₁₁₃N₆O₆) 989.8722, found 989.8718 [M+H]⁺.

### Example 45

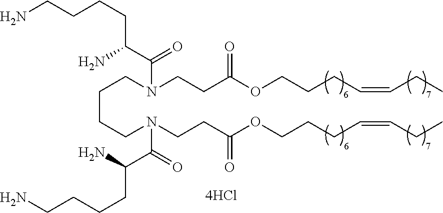

LC-MS (ESI): t_{R}=12.20 min (m/z=989.9 [M+H]⁺ (100%)); HRMS (ESI) m/z calcd (C₅₈H₁₁₃N₆O₆) 989.8722, found 989.8729 [M+H]⁺.

### 5) Gene Delivery

### Description 46

### Plasmid Preparation and Formulations

Plasmid pGL3CMV is a luciferase expression vector based upon pGL3 Basic, (Promega Corporation., Madison, Wis., USA), where the CMV immediate early promoter drives luciferase expression. The plasmid pGL3CMV was used in all gene expression studies in mouse skin biopsies and has been described in WO03/061636.

Plasmid p7313ie is a eukaryotic expression vector where the CMV immediate early promoter drives the production of an encoded antigen (WO03/025003).

Plasmid p7313iTrng, is a vector where the CMV immediate early promoter drives the expression of a fusion antigen, RNG, consisting of parts of the HIV reverse transcriptase, nef and gag genes. The plasmid p7313iTrng was used in all immune response studies in mouse (WO03/025003).

Supercoiled plasmid DNA (low endotoxin) was purified on a large scale, approximately 100 mg yield, to high purity using a combination of alkaline SDS lysis, ultrafiltration and anion exchange column chromatography. Plasmids were resuspended in TE, (10 mM TrisHCl, 1 mM EDTA), pH 8.0 at 1 ug/ul, and determined as >95% supercoiled upon analysis by agarose gel electrophoresis.

Plasmids were formulated in either highly pure Milli Q (Millipore) filtered water after a standard ethanol precipitation procedure (see Chapter 1, Molecular Cloning: A Laboratory Manual, Sambrook, J. et al., 2^{nd} Edition, 1989, CSH Laboratory Press, Cold Spring Harbor, N.Y., USA), or plasmid DNA in TE was directly diluted in Optimem® I (GIBCO Invitrogen). The DNA was re-suspended directly into the aqueous formulation solution at a concentration of 0.4 ug/ul.

DNA formulated for delivery without gemini surfactant, hereinafter “naked DNA”, was formulated in 2×PBS for delivery in vivo through the intradermal route, as this improved gene expression considerably over common aqueous buffered DNA formulation, as has been described (Chesnoy, S & Huang, L (Mole. Therapy (2002) 5: 57-62).

### Description 47

### Gemini Surfactant and Lipoplex Preparation and Formulations

All gemini surfactants were prepared from lyophilised stocks by vortex re-suspension in pure water at a stock concentration of 1 mg/ml stored at 4° C. Gemini surfactants were diluted either in pure water or were directly diluted in Optimem®1 (GIBCO Invitrogen) for immediate use. Where gemini surfactants were formulated together with other helper lipids such as DOPE, these were co-lyophilised at a 1:1, (w/w) ratio and similarly vortex re-suspended in pure water at a stock concentration of 1 mg/ml stored at 4° C.

Gemini surfactants were mixed with plasmid DNA by drop by drop addition of the diluted gemini surfactant formulation (in OPTIMEM) to the diluted plasmid DNA formulation (in OPTIMEM) at room temperature whilst the mixture was being mixed using a Vortex mixer (Mini Vortexer, VWR). The ratio of gemini surfactant to plasmid DNA was varied from 0.2:1 (w/w) to 4:1 (w/w) so that for a final volume of 50 ul per delivery, the formulation contained 10 ug of plasmid DNA with gemini surfactant at the correct ratio. Gemini: DNA mixtures or complexes were left at room temperature for 15 minutes post mixing to form and were then delivered to animals within 15 to 30 minutes. A commercially available cationic lipid, DMRIE-C (Invitrogen Life Technologies) was similarly formulated with DNA.

### Description 48

### PEI and Man-PEI Formulations and Complexes with DNA

The transfection agents, In vivo-jetPEI or In vivo-jetPEI-Man (Polyplustransfection, Qbiogene) were used as per manufacturer's instructions at an N/P ratio of 5. Briefly, complexes were formed with plasmid DNA by drop by drop addition of the diluted PEI (in 5% glucose w/v) to the diluted plasmid DNA formulation (in 5% glucose w/v) at room temperature whilst the mixture was being mixed using a Vortex mixer (Mini Vortexer, VWR). For a final volume of 50 ul per delivery, the formulation contained 10 ug of plasmid DNA with the PEI at the correct ratio. Complexes were left at room temperature for 15 minutes post mixing to form and were then delivered to animals within 15 to 30 minutes.

### Description 49

### In Vivo Delivery by Intradermal Injection and Luciferase Assays

Plasmid DNA was delivered into the skin of Balb/c×C3H F1 female mice. For intradermal (ID) delivery mice were anaesthetised with isofluorane and a 30G hypodermic needle was inserted into a pre-shaved area of skin within the abdomen. 10 ug of plasmid DNA was injected in a 50 ul volume by the standard ID procedure. Groups of 8-10 animals (or 4 to 5 animals with two injection sites) were analysed for skin gene expression. Mice were sacrificed and samples were removed 24 hours post plasmid delivery and snap frozen in liquid nitrogen.

Skin samples were thawed to room temperature and disrupted in 500 ul of Passive Lysis Buffer™ (Promega Corporation, Madison, Wis., USA) using an IKA Labortechnic Ultra turrax T8 polytron. Luciferase enzyme activity was determined using a luciferase assay kit (Promega). 50 μl of the lysate (in duplicate) were assayed together with 250 μl of luciferase assay reagent (Promega) in a 96 well black and white isoplate plate (Wallac, Perkin Elmer). Luciferase activity (RLU) was measured as counts per minute on the Victor 2 1420 multilabel HTS counter on the luminescence program (Wallac, Perkin Elmer). Total protein concentration was calculated by a Coomassie Plus protein assay reagent kit (Perbio) using the manufacturer's protocol. Briefly, 1-5 μl of cell lysate were assayed together with 145-149 μl of water (Sigma) and 150 μl of Coomassie Plus protein assay reagent in 96 well flat-bottomed plates (Costar). The absorbance was measured at 595 nm on a Molecular Devices Spectra Max 340. Luciferase activity was expressed as relative light units (RLU)/mg of total protein.

### Description 50

### Intradermal Electroporation and Gene Gun (PMED) Delivery

Electroporation, where performed, was applied immediately after intradermal DNA injection using the BTX (Genetronics) T830 squarewave electroporator with 1 cm×1 cm caliper electrodes, as per manufacturer's instructions. Briefly the electrodes were used with a 1 mm gap device using the following conditions: 100V applied for 20 msecs three times with a current reversal and a further three pulses, the interpulse delay was 10 secs.

Preparation of cartridges for gene gun, particle-mediated epidermal delivery (PMED) with the XR1 gene transfer device (PowderMed) was as previously described (Eisenbraun et al., DNA and Cell Biology (1993) 12(9):791-797; Macklin et al., in ‘Methods in Molecular Medicine, vol 29, DNA Vaccines: Methods and Protocols, ed. Lowrie, D. and Whalen, R., (2000) Humana Press Inc., USA). Briefly, plasmid DNA was coated onto 2 μm gold particles (DeGussa Corp., South Plainfield, N.J., USA) and loaded into Tefzel tubing, which was subsequently cut into 1.27 cm lengths to serve as cartridges and stored desiccated at 4° C. until use. In a typical vaccination, each cartridge contained 0.5 mg gold coated with a total of 0.5 μg DNA/cartridge. Plasmid was administered by particle mediated gene transfer (0.5 μg DNA/cartridge) into the skin of mice. Plasmid was delivered to the shaved target site of abdominal skin of Balb/C mice (purchased from Charles River United Kingdom Ltd, Margate, UK) from one cartridge using the XR1 gene transfer device at 500 lb/in² (WO 95/19799).

### Example 46

### Gemini Surfactants Facilitate Gene Delivery and/or Gene Expression in Mouse Skin

Gemini surfactants: GSC103-L-Lys+/−DOPE (+/− means with or without DOPE), GSC170-Lys and GSC170-Orn that showed enhanced DNA transfection activity in cell lines in vitro (Castro, M et al (2004) Org. Biomol. Chem. 2:2814-2820) were tested for their ability to enhance DNA transfection in vivo after intradermal injection into mouse skin. Gemini surfactants and DNA were diluted in OPTIMEM to a final volume of 50 ul per injection containing 10 ug of pGL3CMV plasmid DNA with gemini:DNA ratios as follows:

### GSC103-L-Lys 0.5:1

### GSC103-L-Lys+DOPE 0.4:1, 0.5:1 and 0.6:1

### GSC170-Lys 0.5:1

### GSC170-Orn 0.5:1

prior to intradermal delivery in to mouse skin. The effect on gene expression was then measured as luciferase activity (relative units/mg protein) and compared to that obtained from background, untreated mouse skin (“negative”), naked DNA (“DNA”) and the alternative transfection agents DMRIE-C, JetPEI and JetPEI-Man and alternative DNA delivery methods BTX CE (electroporation) and PMED (“gene gun”) all delivering 10 ug of plasmid apart from PMED, where 0.5 ug plasmid DNA was delivered. The results are shown in FIGS. 1 and 2.

FIG. 1 shows that GSC103-L-Lys+DOPE at gemini:DNA ratios of 0.5:1 enhances gene delivery and/or expression in mouse skin over naked DNA alone.

FIG. 2 shows that GSC103-L-Lys+DOPE formulated in OPTIMEM with DNA, at ratios of 0.4:1, 0.5:1 and 0.6:1 enhance gene delivery and/or expression over naked DNA alone. The DOPE co-lipid formulation appears not to be essential for the enhanced effect.

### GSC103-L-Lys (Spermine Based Gemini—See WO00/77032)

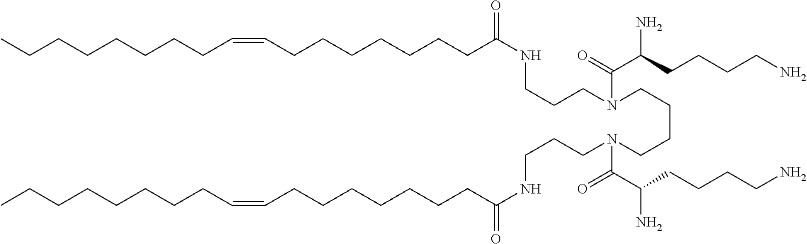

### GSC170-Lys (WO03/82809)

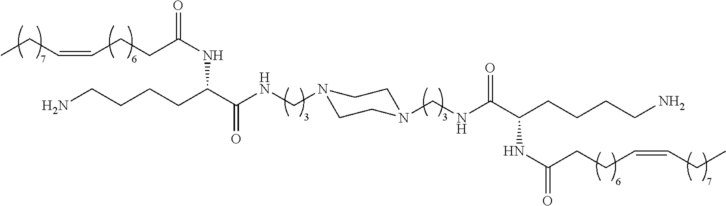

### GSC170-Orn (WO03/82809)

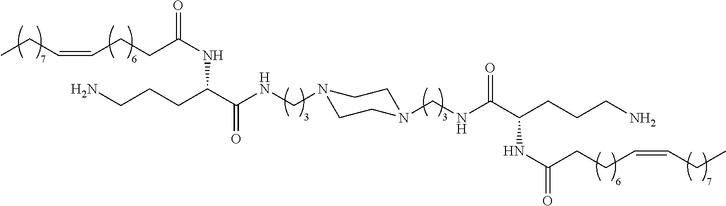

### Example 47

### Other Classes of Gemini Surfactants Facilitate Gene Delivery and/or Expression in Mouse Skin

A further experiment was performed to evaluate the enhancement of gene delivery and/or expression over naked DNA for intradermal delivery of a range of different classes of gemini surfactants: GSC103-L-Lys+/−DOPE (spermine-based), GS064A (spermidine-based), GS062A (spermidine-based), GSC103-L-Lys-oleic acid/stearic acid unsymmetrical (GSC103-L-Lys,Ol,St), GSC103-D-Lys-oleic acid/stearic acid unsymmetrical (GSC103-D-Lys,Ol,St), GSC103-L-dab-oleic acid/oleic acid (GSC103-L-dab,Ol,Ol), GSC103-L-Orn-oleic acid/stearic acid unsymmetrical (GSC103-L-Orn,Ol,St), formulated in OPTIMEM, at the ratios of 0.5:1 (w/w gemini: DNA). Data from this experiment is shown in FIG. 3.

The data shows that all the gemini surfactants with the exception, in this experiment, of GSC103-L-Lys+DOPE and GSC103-D-Lys-oleic acid/stearic acid unsymmetrical, facilitated higher levels of gene delivery and/or expression than DNA similarly formulated with the commercial lipid DMRIE-C or when boosted by electroporation.

### GS064A (Example 25)

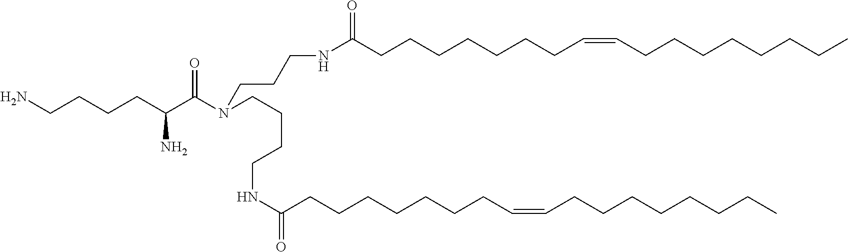

### GS062A (Example 24)

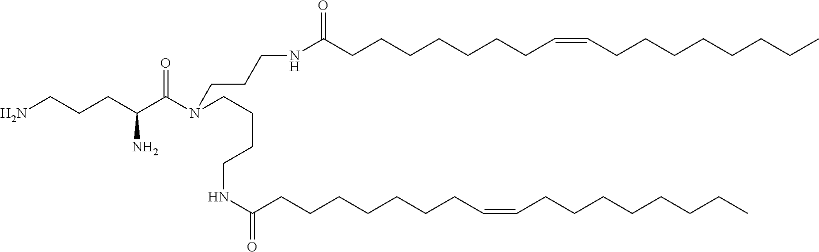

### GSC103-L-Lys Oleic Acid, Stearic Acid Unsymmetrical (Example 1)

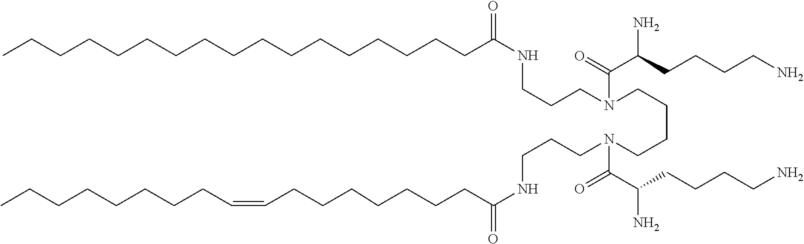

### GSC103-D-Lys Oleic Acid, Stearic Acid Unsymmetrical (Example 2)

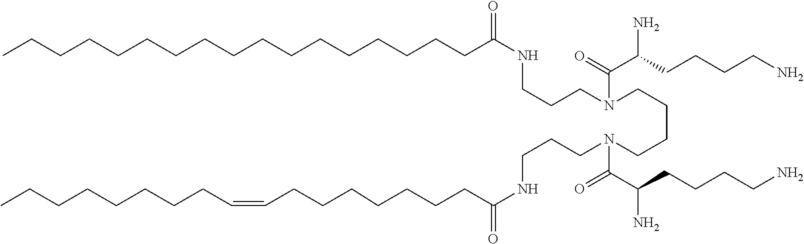

### GSC103-L-dab Oleic Acid, Oleic Acid (WO00/77032)

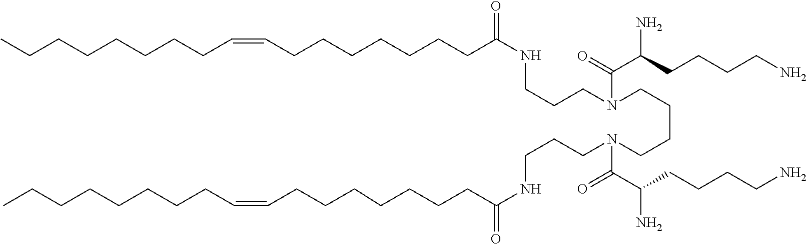

### GSC103-L-orn Oleic Acid, Stearic Acid (Example 3)

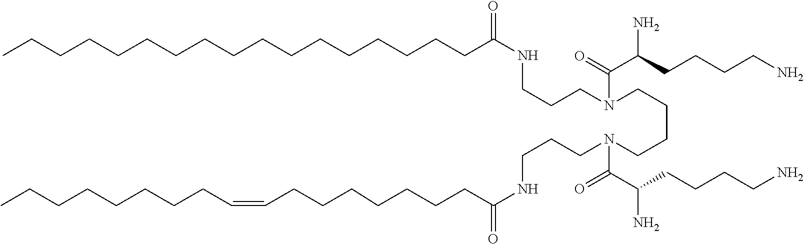

### Example 48

### Gemini Surfactants Delivered Intradermally into Mouse Skin can Enhance Cellular and Humoral Immune Responses Over Naked DNA to Plasmid Encoded Antigen

### A) ID Immunisation

Acclimatised 6-8 week old Female Balb/c mice were maintained under general anaesthesia using an oxygen-controlled inhaled Isoflourane mask for application by intradermal injection to the pre-shaved lower back above the base of the tail. Where necessary mice were given Rimadyl (Carprofen) as an analgesic in a sub-cutaneous dose of 5 mg/Kg, diluted 1:10 and delivered at 20 μl/mouse. Injections of up to 50 μl of freshly formulated DNA with or without gemini surfactant were given via BD 0.5 ml insulin syringes with 29 g needles.

### B) Analysis of Cytotoxic T Cell Responses by ELISPOT

Mice were killed by cervical dislocation and spleens were collected into ice-cold PBS. Splenocytes were teased out into phosphate buffered saline (PBS) followed by lysis of red blood cells (1 minute in buffer consisting of 155 mM NH₄Cl, 10 mM KHCO₃, 0.1 mM EDTA). After two washes in PBS to remove particulate matter the concentration of cells in the single cell suspension was assessed by counting in a Guava Flow Cytometer. Cells were aliquoted into ELISPOT plates previously coated with capture IFN-γ or IL2 antibody and stimulated with CD8 or CD4-restricted cognate peptides in culture medium (RPMI 1640 plus 10% heat inactivated foetal calf serum and appropriate antibiotics). After overnight culture, IFN-γ or IL2 producing cells were visualised by application of anti-murine IFN-γ-biotin or IL2-biotin labelled antibody (Pharmingen) followed by streptavidin-conjugated alkaline phosphatase and quantitated using image analysis.

### C) Analysis of Humoral Responses by ELISA

The humoral response was assessed by measuring antigen-specific whole IgG antibody levels (eg. to HIV RNG fusion protein) in the serum samples collected after primary and boost immunisation. Microtitre plates (Nunc Immunoplate F96 maxisorp, Life Technologies) were coated with 10 μg/ml antigen by overnight incubation at 4° C. and washed 4 times with washing buffer (PBS containing 5% Tween 20). This was followed by a 1 hour incubation at 20° C. with serum samples serially diluted in blocking buffer. After 4 further washes (as above) to remove unbound antibody, plates were incubated for 1 hour with peroxidase conjugated anti-mouse IgG antibody (AMS Southern Biotechnology) diluted in blocking buffer. The amount of bound antibody was determined after 4 further washes (as above) followed by addition of TMB substrate solution (T-8540-Sigma). After 30 minutes at 20° C. protected from light, the reaction is stopped with 1M sulphuric acid and absorbance read at 450 nm. Titres are defined as the highest dilution to reach an OD of 0.2.

### Gemini Surfactants Tested

### GS092A-Ester Linked Dab (Example 42)

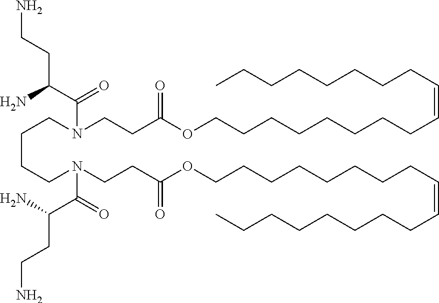

### GS543A-Pentamine-Based L-Orn (Example 16)

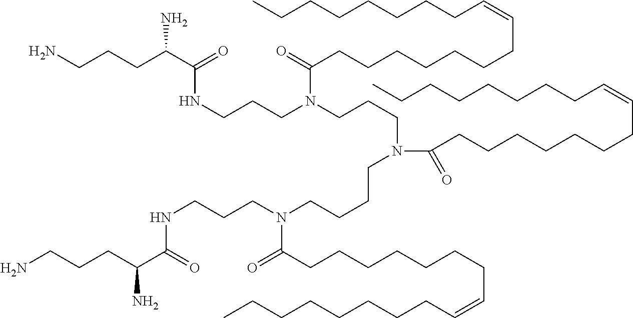

Representatives of 3 classes of gemini surfactants were assessed for their ability to induce both cellular and humoral immune responses upon delivery to mouse skin through the intradermal route. The examples and classes of gemini surfactants that were evaluated in this experiment were: GSC103-L-Lys (spermine-based), GS092A (ester-linked), GS064A (spermidine-based) and GS543A (pentamine). The immunization regime used for this experiment involved priming the mice with DNA by PMED (FIG. 4) and then leaving them for 140 days for the primary immune responses to fall to background levels. The comparative immune responses after a heterologous boost, were then evaluated for cellular responses (FIGS. 5-10) at the following time points post-boost, day 8, 14 and 21, by interferon-γ ELISPOT (FIGS. 5, 7 and 9) and by interleukin-2 ELISPOT (FIGS. 6, 8 and 10). All gemini compounds tested in this experiment demonstrated increased cellular responses compared to those induced by injecting naked DNA alone.

Humoral responses to RNG fusion peptide (WO03/025003), as serum whole IgG, were monitored at day 14 post-boost (FIG. 11). An increase in antibody levels in immunised mice treated with gemini surfactant in the formulation over those immunised without gemini surfactant in the formulation indicates an enhancing effect of gemini surfactant on humoral responses.

The levels of immune responses, both cellular and humoral (FIGS. 5 to 11) induced by intradermal delivery of gemini surfactants, (including GSC103-L-Lys), are comparable to those induced by PMED at boost. Relative immune response levels are much closer between gemini surfactants, (eg. GSC103-L-Lys), than the relative gene expression levels, (eg. compare GSC103-L-Lys v PMED, see FIGS. 2 and 3).

Therefore gemini surfactants benefit the immune responses to DNA vaccination considerably more than would be predicted from in vivo DNA transfection enhancement alone and therefore demonstrate an additional adjuvant effect.

### Description 51

### Plasmid Preparation and Formulation for Lipoplex Characterisation, In Vitro

Plasmid pdpSC18 is a vector where two copies of the CMV immediate early promoter separately drive the expression of the Hepatitis B core and Surface antigens (S-Ag), (WO0236792). Plasmid pdpSC18 was used as a ‘generic plasmid’ for the optimisation of Gemini Surfactant and plasmid DNA complexation studies.

### Description 52

### Determination of Particle Size by Quasi-Elastic Light Scattering (QELS)

The size of the particles formed by gemini surfactants and their complexes with plasmid DNA was measured by quasi-elastic light scattering (QELS) (Finsy, R., Advances in Colloid and Interface Science (1994) 52 (19 September):79-143; Gittings, M. R., and Saville, D. A., Colloids and Surfaces A: Physicochemical and Engineering Aspects (1998) 141: 111-117). Samples (50 μl) were analysed using a Brookhaven Instruments Corporation particle size analyser (BIC 90 Plus) following the manufacturer's instructions: 10 measurements, each consisting of 10 runs were taken per sample. The raw light scattering data was automatically collected and manipulated by the instrument using the Stokes-Einstein equation. It was then expressed in the form of correlogram, lognormal particle distribution and multimodal particle distribution. The particle distributions were plotted as an expression of light scattering intensity as well as particle number. The average particle size was automatically derived from the raw data by the instrument and expressed as the mean hydrodynamic diameter in nanometers (nm).

### Example 49

### Stability of Gemini Surfactants Upon Storage at 4° C. as an Aqueous Liquid

In order to provide a measure of the stability of the gemini surfactants upon storage, the colloidal properties of gemini surfactants stored in aqueous solution were investigated. Freshly prepared gemini surfactant solutions (1 mg/ml in water) were compared to surfactant solutions that had been stored at 4° C. for 4 months. The comparison was performed by detecting the formation of any colloidal particles by QELS. The comparison revealed that the surfactants were unstable upon storage, as they seemed to gradually form particles. Fresh gemini surfactant solutions were found to contain no particulate structures, whereas the stored solutions contained particles that were detectable by QELS (FIG. 27). This, however, was not the case with GS543A, which exhibited particle formation both in the fresh and stored suspensions. However, the size of the particles did not differ significantly between the fresh and stored solutions suggesting colloidal stability in this case.

The colloidal properties of gemini surfactant solutions were monitored over time in order to analyse their apparent instability in greater detail. Fresh solutions of the gemini surfactants were stored at 4° C. and the particle size was measured at bi-weekly intervals. The results obtained following a 10-week study are shown in FIG. 28. As the figure shows, all gemini surfactant solutions were stable for the first 2 weeks of the study. Following 2 weeks of storage, the surfactants GS064A and GSC103-L-Lys began to form particulate structures. The gemini surfactant GS092A remained stable until week 6, before displaying particle formation. The solution of the surfactant GS543A exhibited small particles from the beginning of the study. The particles seemed to remain stable throughout the storage period, suggesting that GS543A is stable as a colloid under the storage conditions investigated.

### Description 53

### Determination of Particle Zeta Potential by Electrophoretic Light Scattering (ELS)

The surface charge of the particles formed by gemini surfactants and their DNA complexes was determined by electrophoretic light scattering (ELS) (McNeil-Watson et al., Colloids and Surfaces A: Physicochemical and Engineering Aspects (1997) 140: 53-57; Gittings, M. R., and Saville, D. A., Colloids and Surfaces A: Physicochemical and Engineering Aspects (1998) 141: 111-117). The zeta potential was measured in a variety of media using a Brookhaven Instruments Corporation particle size analyser (BIC 90 Plus, zeta potential function, reference beam mode). Each sample was analysed by a series of 10 automated measurements, with the surface charge expressed as zeta potential in millivolts (mV). When required, the instrument was used to determine the pH of lipoplex suspensions and relevant media, by means of a glass electrode.

### Example 50

### Gemini Surfactants Form Complexes with Plasmid DNA

The ability of the gemini surfactants to form complexes with plasmid DNA was investigated by mixing suspensions of the surfactants in Optimem® I medium (GIBCO Invitrogen) with solutions of plasmid DNA (pdpSC18) in the same medium. The complexes were formed following the procedure in Description 47. In order to confirm the formation of complexes, the mixtures were analysed by QELS. As FIG. 29 shows, all gemini surfactants tested were able to form complexes with the plasmid DNA, resulting in nanoparticles that were larger than the colloidal formations of the gemini surfactants alone, when measured in OPTIMEM medium.

### Example 51

### Gemini Surfactant Complexes with Plasmid DNA Possess a Negative Surface Charge

The formation of complexes of the gemini surfactants with plasmid DNA (pdpSC18) was confirmed by measuring their surface charge by ELS in dilute OPTIMEM medium. All samples were diluted three-fold in water to prevent any interference from the OPTIMEM medium. The gemini surfactant-DNA complexes were found to be negatively charged, unlike free gemini particulate formations, which were cationic (FIG. 30). This reduction in the surface charge between complexes and free surfactant is due to association with the negatively charged plasmid DNA.

### Example 52

### Gemini Surfactants Form Stable Complexes within Minutes of Plasmid DNA Addition

The stability of the gemini surfactant complexes with plasmid DNA was investigated from the point of complex formation until the time point of in vivo delivery, which is typically up to 50 minutes after the complexation reaction. The purpose of the study was to confirm that the particle size of the complexes does not change significantly from the point of formation until in vivo administration.

Gemini surfactants were complexed with plasmid DNA (pdpSC18) in OPTIMEM and the particle size was monitored by QELS at 10-minute intervals. The results obtained are shown in FIG. 31. As the results in indicate, the particle size of the gemini-DNA complexes does not change significantly over the time period investigated, suggesting that all gemini surfactants rapidly form and then maintain stable complexes until the point of in vivo administration.

### Example 53

### Effect of Gemini Surfactant to DNA Ratio on Complex Size

The range of gemini surfactant ratios that could be used to form complexes with plasmid DNA (pdpSC18) was investigated. Complexes were formed in OPTIMEM at gemini surfactant to DNA ratios ranging from 0.5:1 to 8:1 (w/w), and their size was determined by QELS. The gemini surfactant used in the study was the pentamine GS543A, which generated the results presented in FIG. 32. The results obtained suggest that the surfactant is capable of forming stable lipoplexes of small size (less than 200 nm) at up to a ratio of 2:1 (w/w). At greater gemini surfactant to DNA ratios (more than 4:1 w/w) the resulting complexes were unstable, resulting in the formation of large aggregates that exceeded 1 μm in diameter, which are not preferred for delivery in vivo. Therefore, the surfactant GS543A can potentially be used for complexation with plasmid DNA at ratios up to 2:1 (w/w) for in vivo delivery.

### Example 54

### Investigation of the pH Sensitivity of Gemini Surfactant-DNA Complexes

The efficiency of the gemini surfactants as DNA delivery systems is thought to be dependent on their pH sensitivity (Fielden et al., European Journal of Biochemistry (2001) 268: 1269-1279; Kirby, A. J. et al., Angew. Chem. Int. Ed. (2003) 42: 1448-1457). Sensitivity to pH that triggers conformational changes of the lipoplex, most commonly in the form of micelle formation, is thought to be required for efficient delivery of the plasmid DNA load (Fielden et al., European Journal of Biochemistry, (2001) 268: 1269-1279). It allows escape of the plasmid DNA from the acidic endosomal compartment to the cell cytoplasm, where it is safe from degradation and can reach the cell nucleus (Zabner et al., Journal of Biological Chemistry (1995), 270: 18997-19007; Xu and Szoka, Biochemistry (1996) 35: 5616-5623; Singh et al., Chemistry & Biology (2004) 11 (May): 713-723). In order to determine the pH sensitivity of the lipoplexes formed by gemini surfactants, their ability to form micelles (characterised by small particle size) at acidic pH was investigated. Gemini surfactant-DNA complexes were formed and then incubated in buffers that covered the range of pH of the endosome, from the early endosome stage (pH 6.5) until the late endosome-lysosome stage (pH 4.5) (Simões et al., Advanced Drug Delivery Reviews (2004) 56: 947-965). The response of the lipoplexes to the changing pH was measured in the form of particle size, in order to detect the formation of micelles.

The results of the study are shown in FIG. 33. All gemini surfactants were found to form lipoplexes that displayed pH sensitivity, however the pattern of pH sensitivity differed between the surfactants. In general, the particle size fluctuated with falling pH, particularly in the case of GSC103-L-Lys (FIG. 33). When exposed to the lowest pH, all lipoplexes underwent a drop in particle size. The size reduction was most pronounced in the case of GS092A and GS543A, which displayed particles of a mean hydrodynamic diameter of 9 nm. This size is small enough to suggest the formation of micellar particles (Fielden et al., European Journal of Biochemistry (2001) 268: 1269-1279; Asokan and Cho, Biochimica et Biophysica Acta (2003) 1611: 151-160). The data suggests that the gemini surfactants GS092A and GS543A may form micelles in this study at pH 4.3.

### Example 55

### Effect of Complexation Medium on Lipoplex Size

Since the gemini surfactants were found to be pH sensitive, it was necessary to investigate the effect of different media on the colloidal properties of the surfactants. The work described here compares the colloidal properties of the gemini surfactants in water and OPTIMEM. Gemini surfactant solutions were analysed by QELS in water (1 mg/ml) and the aqueous solutions were then diluted in OPTIMEM in the same manner as in preparation for a complexation reaction. The resulting solutions were then analysed by QELS and the results compared (FIG. 34).

The results suggest that the gemini surfactants do not form any colloidal particles in water, with the exception of GS543A. In contrast, all surfactants form particles in OPTIMEM medium. In the case of GS543A, the particles were of a larger size than those in water.

The OPTIMEM medium used in the complexation reaction is bicarbonate buffered, and as a result its pH gradually increases with storage as the medium is exposed to air and its equilibrium is disturbed, (data not shown). The pH of a freshly opened bottle of OPTIMEM medium was found to be 7.17, whereas that of a bottle opened 3 months previously, (‘old’), had increased to 7.68. Since the gemini surfactants were found to be sensitive to pH changes, it was necessary to investigate the effect of the pH variability of OPTIMEM on lipoplex formation. Both old (3 months), and new, (freshly opened bottle), OPTIMEM was used as the medium to form lipoplexes between gemini surfactants and plasmid DNA (pdpSC18). The particle size of the resulting lipoplexes was then measured by QELS. The results of the comparison are shown in FIG. 35. The increase in the pH of the OPTIMEM medium was found to greatly affect the size of the lipoplexes that were formed, with the exception of GS543A, where the lipoplex size was not significantly affected.

In general, the OPTIMEM medium was found to be unstable, with its instability resulting in lipoplexes of variable particle size. An additional disadvantage of using OPTIMEM is that it contains animal-derived substances and is thus unsuitable for product development. Further work thus focused on replacing it with an alternative medium that was suitable for development and more stable. A series of potential buffers were selected for testing:
1. Earle's Balanced Salts Solution (EBSS) from Invitrogen Corporation
2. Tris Buffered Saline (TBS) from Sigma Aldrich
3. N-(2-Hydroxyethyl)piperazine-N′-(2-ethanesulfonic acid) (HEPES) buffer from Sigma Aldrich
4. Phosphate Buffered Saline (PBS) from Invitrogen Corporation

EBSS was selected for being the closest alternative to OPTIMEM. It is similar to OPTIMEM in its salt and bicarbonate content, and thus similarly unstable in terms of pH. However, EBSS does rapidly stabilise its pH upon exposure to air, for example a 2 ml volume stabilised from pH 7.4 to pH 8.0-8.1 upon 10 minutes exposure to air, (data not shown). EBSS is superior to OPTIMEM in that it is free from animal-derived products and as such suitable for development. The remaining buffers were chosen for having the additional advantage of pH and storage stability.

Lipoplexes were formed following the same procedure as with OPTIMEM and using the gemini surfactant GS543A and the plasmid pdpSC18 for complexation. The particle size of the resulting complexes was then analysed by QELS. The results obtained are shown in FIG. 36. As the results show, lipoplexes were successfully formed in all buffers. The particle size of the lipoplexes did not differ substantially between media. The largest sizes were obtained with OPTIMEM, TBS and EBSS (113, 109 and 108 nm respectively) whereas HEPES and PBS appeared to give smaller complexes (87 and 96 nm respectively).

### Example 56

### Preparation and Analysis of Gemini Surfactant

### Plasmid DNA Lipoplexes by Agarose Gel Electrophoresis

Gemini Surfactant and plasmid DNA lipoplexes were prepared as in Description 47. The ratio of gemini surfactant to plasmid DNA was kept at 0.5:1 (w/w), the buffer was varied from OPTIMEM, (OPT), to be either EBSS, (Invitrogen Corporation) or 1×PBS, pH 7.2, (without CaCl2 and MgCl2, Invitrogen Corporation). 10 ul (1 ug DNA), or 20 ul, (2 ug DNA), aliquots of lipoplex were analysed on an agarose gel and compared to 1 ug of plasmid DNA in buffer without gemini surfactant. Agarose gel electrophoresis was performed on 1.2% Agarose E-Gels®, (Invitrogen Corporation), following the manufacturer's instructions and using an E-Gel PowerBase, (Invitrogen Corporation), for 20 to 30 minutes. DNA and lipoplexes were visualised under u.v. light using an Epi Chemi II Darkroom and ccd camera based gel imaging system, (UVP Laboratory Products). Images were analysed using the Lab Works™ Bioimaging System (UVP).

An example of such an analysis is shown in FIG. 37, where gel retardation of 1 ug of plasmid DNA can be seen in the presence of GS543 using OPTIMEM, (compare lane 1 and lane 2), EBSS, (compare lane 4 and lane 5), and PBS, (compare lane 7 and lane 8), to buffer the lipoplex formation. Gel retardation has been used as a standard assay to confirm both lipoplex and polyplex formation, respectively, between plasmid DNA and cationic lipids or cationic polymers, (Huang, C Y et al., Chemistry & Biology 5, 345, 1998; Kim, Y H et al., J. Controlled Release 103, 209, 2005).

### Example 57

### Analysis of Stored and Fresh Batches of Gemini Surfactants for Ability to Enhance Cellular Immune Responses Over Naked DNA to Plasmid Encoded Antigen

Gemini Surfactant stocks were prepared as in Description 47. Stocks described as ‘old’ for this example had been stored as a liquid 1 mg/ml stock for 77 days at 4° C. Stocks described as ‘new’ for this example were prepared fresh 1 day prior to administration and were stored overnight at 4° C.

Gemini Surfactant and plasmid DNA lipoplexes were prepared as in Description 47 and administered intradermally (ID) to Balb/c mice as described in Example 48 (A). As in Example 48 (A) a similar immunisation regime was used involving priming the mice with DNA by PMED, see FIG. 4, however the time interval between prime and boost in this example was 50 days. Mice were boosted with either naked DNA or PMED, or PBS as a negative control, and responses were compared to those generated after boosting with lipoplexes made using ‘old or ‘new’ stocks of three classes of Gemini Surfactants. As in Example 48, comparative cellular immune responses were evaluated at day 7/8, day 14 and day 21, post boost. An example of the data from this experiment is shown in FIG. 38, (Interferon γ ELISPOT at day 14 post boost), and in FIG. 39, (Interleukin-2 ELISPOT at day 14 post boost).

The data shows that GS543A: DNA lipoplexes formed using either freshly prepared, (‘new’) or stored, (‘old’) GS543A stocks, generated very similar cellular immune responses upon immunisation of mice via the intradermal route.

### Example 58

### Analysis of Stored and Fresh Batches of GS543A for Ability to Enhance Cellular Immune Responses when GS543:DNA Lipoplexes are Buffered Using OPTIMEM or EBSS

Gemini Surfactant stocks were prepared as in Description 47. Stocks described as (FEB) for this example had been stored as a liquid 1 mg/ml stock for 289 days, (>9 months) at 4° C. Stocks described as (SEP) for this example had been stored for 2 months at 4° C.

Gemini Surfactant and plasmid DNA lipoplexes were prepared as in Description 47 for samples labelled OPT in this example, (ie. buffered with OPTIMEM). Gemini Surfactant and plasmid DNA lipoplexes were also prepared using EBSS as described in Example (55) for samples labelled EBSS in this example. Complexes were administered intradermally (ID) to Balb/c mice as described in Example 48 (A). As in Example 48 (A) a similar immunisation regime was used involving priming the mice with DNA by PMED, see FIG. 4, however the time interval between prime and boost in this example was 168 days. Mice were boosted with either lipoplexes or PMED, or OPTIMEM as a negative control, and responses were compared for lipoplexes formed from different GS543A stocks and using different buffers: OPTIMEM or EBSS. As in Example 48, comparative cellular immune responses were evaluated at day 7/8, day 14 and day 21, post boost. An example of the data from this experiment is shown in FIG. 40, (Interferon γ ELISPOT at day 14 post boost), and in FIG. 41, (Interleukin-2 ELISPOT at day 14 post boost).

The data shows that GS543A: DNA lipoplexes formed using different stocks, even prepared from stocks stored for greater than 9 months at 4° C., and prepared using either of the buffers: OPTIMEM or EBSS, generated very similar cellular immune responses upon immunisation of mice via the intradermal route.

### Example 59

### GS543A Delivered Intradermally into Mouse Skin can Enhance Immune Responses Over Naked DNA to Plasmid Encoded Antigen in a Homologous Administration Regime and Acts to Prime and Enhance Responses at Boost to Adenovirus Encoded Antigen

### A) Intramuscular Injection

Plasmid DNA administration by intramuscular, (i.m./IM) dosing was performed using a 0.5 ml insulin syringe with 0.33 mm (29 g)×12.7 mm needle (BD Micro-Fine). Two doses of 50 μl were administered at two separate sites on the hind limbs of a Balb/c mouse on each occasion. The muscle was chosen from the biceps femoris and the quadriceps and alternated between each respectively at each subsequent immunisation. The required volume of formulated vaccine was drawn into each syringe and any air bubbles removed by flicking. Injections of 50 μl were over 5 seconds and were made by inserting the needle 3-4 mm into the muscle at a shallow angle. If on occasion minor bleeding from surface capillaries occurred, this was stopped by pressure prior to returning the animal to its cage.

### B) Tetramer Staining Using the Q Preparation Protocol

Tri-sodium-citrate (Merck 102424L) was dissolved in water to give a 3.8%, (w/v), solution. 1000 was placed in labelled 1.5 ml Eppendorf tubes. A 50-100 ul blood sample was collected from the lateral tail vein of pre-warmed experimental mice and transferred to relevant tubes. Tubes were agitated to mix and prevent clotting. A set of Flow Cytometry tubes, (Starstedt), were labeled and 10 μl H2-Kd HIV Gag (AMQMLKETI) tetramer conjugated to Phycoerythrin (Beckman Coulter custom synthesis T04005, 1 μg/10 μl) was added to each tube, (except for a control tube). 100 μl of whole blood mixed with citrate buffer was added to each tube. Tubes were incubated at room temperature for 20 mins in the dark. 10 μl of a 1/20 dilution of 0.2 mg/ml anti-mouse CD8-CyChrome (CD8a cychrome anti-mouse (Ly-2), (53-6.7) (BD Pharmingen 553034) was added per tube, (including the control). Tubes were incubated for a further 15 minutes in the dark. 50 ul of heated inactivated foetal calf serum were added per tube. The tubes were then placed on a Beckman Coulter TQ Prep and labelled cells were lysed/fixed using the Immunoprep reagent system (Beckman Coulter 7546999). 3 ml of FACS buffer, (2.5% heat inactivated foetal calf serum in Phosphate Buffered Saline) was added to each tube which was then centrifuged for 5 minutes at 1500 rpm and the supernatant was discarded. The tubes were vortexed to mix and the pellet was resuspended in 500 μl FACS buffer. The percentage of tetramer positive CD8 cells was determined by analysis using EXPO 32 ADC software in a standardised protocol using a Beckman Coulter Epics XL-MCL Flow Cytometer.

### C) Non-Human Primate Adenovirus Stocks and Administration.

An E1/E3-deleted non-human primate (NHP)-derived adenoviral vector, of serotype Pan 6 (also known as C6) comprising a polynucleotide encoding the HIV antigens Gag, RT and Nef under the control of the CMV immediate early promoter (described in WO02/36792) was used for heterologous boost immunisation in this example. This vector construct has been described in PCT/EP2006/004854 and the antigens described in WO03/025003. Details of how these vector constructs were made are set out in Examples 60 to 62.

Gemini Surfactant stocks and plasmid DNA lipoplexes were prepared as in Description 47 for samples labelled OPT in this example, (ie. buffered with OPTIMEM). Gemini Surfactant and plasmid DNA lipoplexes were also prepared using EBSS or PBS as described in Example (55). Complexes were administered intradermally (ID) to Balb/c mice as described in Example 48 (A) or intramuscularly (IM) as described above. The immunisation regime used for this example involved priming the mice either with naked DNA or GS543A: DNA lipoplexes and then following this with a homologous boost of a repeat primary immunisation followed by a heterologous boost with 1×10E8 particle forming units (p.f.u), of Pan6 NHP GRN Adenovirus, (p6grn), see FIG. 42.

Mice were primed and boosted either with lipoplexes or naked DNA or PBS as a negative control, and the prime and boost interval was 28 days, see FIG. 42.

Responses were compared for lipoplexes formed from different buffers: OPTIMEM, (OPT) or EBSS or PBS, initially at day 10 post boost by ELISPOT. Comparative cellular immune responses were evaluated at day 10 post boost. The data from this experiment is shown in FIG. 43, (Interferon γ ELISPOT), and in FIG. 44, (Interleukin-2 ELISPOT). The data shows the increased effectiveness of the i.d. over the i.m. route in generating cellular responses in this system. The data demonstrates that GS543A: DNA lipoplexes, formed using EBSS particularly, generate greater cellular immune responses than immunisation of mice with naked DNA alone, especially via the intradermal route, in a homologous immunisation regime.

The resting levels of cellular immune response after the primary immunisation were measured at day 26 post prime by identifying the percentage of Gag positive CD8 T cells present in the blood. The data for this is shown in FIG. 45. The percentage of Gag positive CD8 T cells present in the blood were also measured again at day 26 post 1^{st} boost and the data is shown in FIG. 42 (day 54). The difference in levels of Gag positive CD8 T cells found in the blood, post 1^{st} boost compared to post prime, for animals that had received GS543A: DNA lipoplexes, which had been complexed with EBSS and delivered via the i.d. route, were again increased over those that had received naked DNA alone.

Cellular responses were also analysed again after a heterologous boost with NHP Adenovirus p6GRN which expresses a very similar antigen to that expressed by p73iTrng. An example of such data, obtained at day 7 post heterologous boost, see FIG. 42, is shown in FIG. 47, (Interferon γ ELISPOT), and in FIG. 48, (Interleukin-2 ELISPOT). The data confirms that priming mice with two immunisations of GS543A: DNA lipoplexes complexed with EBSS provides an advantage in terms of enhanced cellular responses after boosting with NHP Adenovirus expressing a similar antigen, especially through the intradermal route. An advantage over priming with naked DNA alone has been demonstrated using GS543A: DNA lipoplexes complexed with EBSS and an advantage of primary responses generated from immunisation with NHP Adenovirus alone.

Humoral responses were also analysed in this example. The humoral responses were measured as whole serum IgG and data were collected both after a homologous prime and boost and again after a heterologous boost with NHP Adenovirus p6GRN, see FIG. 42. An example of such data, obtained at day 10 post homologous boost is shown in FIG. 49, and that obtained at day 14 post heterologous boost is shown in FIG. 50. The data shows that both after a homologous and a heterologous boost, mice that initially received lipoplexes of GS543A: DNA buffered with EBSS, through the intradermal route, generated the strongest humoral as well as the strongest cellular responses.

### Example 60

### Construction of the E1/E3 Deleted Pan 6 and 7 Adenovirus

### 1. Generation of Recombinant E1-Deleted SV-25 Vector

A plasmid was constructed containing the complete SV-25 genome except for an engineered E1 deletion. At the site of the E1 deletion recognition sites for the restriction enzymes I-CeuI and PI-SceI which would allow insertion of transgene from a shuttle plasmid where the transgene expression cassette is flanked by these two enzyme recognition sites were inserted.

A synthetic linker containing the restriction sites SwaI-SnaBI-SpeI-AflII-EcoRV-SwaI was cloned into pBR322 that was cut with EcoRI and NdeI. This was done by annealing together two synthetic oligomers SV25T (5′-AAT TTA AAT ACG TAG CGC ACT AGT CGC GCT AAG CGC GGA TAT CAT TTA AA-3′) and SV25B (5′-TAT TTA MT GAT ATC CGC GCT TAA GCG CGA CTA GTG CGC TAC GTA TTT A-3′) and inserting it into pBR322 digested with EcoRI and NdeI. The left end (bpi to 1057) of Ad SV25 was cloned into the above linker between the SnaBI and SpeI sites. The right end (bp 28059 to 31042) of Ad SV25 was cloned into the linker between the AflII and EcoRV sites. The adenovirus E1 was then excised between the EcoRI site (bp 547) to XhoI (bp 2031) from the cloned left end as follows. A PCR generated I-CeuI-PI-SceI cassette from pShuttle (Clontech) was inserted between the EcoRI and SpeI sites. The 10154 bp XhoI fragment of Ad SV-25 (bp2031 to 12185) was then inserted into the SpeI site. The resulting plasmid was digested with HindIII and the construct (pSV25) was completed by inserting the 18344 bp Ad SV-25 HindIII fragment (bp11984 to 30328) to generate a complete molecular clone of E1 deleted adenovirus SV25 suitable for the generation of recombinant adenoviruses. Optionally, a desired transgene is inserted into the I-Ceul and PI-SceI sites of the newly created pSV25 vector plasmid.

To generate an AdSV25 carrying a marker gene, a GFP (green fluorescent protein) expression cassette previously cloned in the plasmid pShuttle (Clontech) was excised with the restriction enzymes I-Ceul and PI-SceI and ligated into pSV25 (or another of the Ad chimp plasmids described herein) digested with the same enzymes. The resulting plasmid (pSV25GFP) was digested with SwaI to separate the bacterial plasmid backbone and transfected into the E1 complementing cell line HEK 293. About 10 days later, a cytopathic effect was observed indicating the presence of replicative virus. The successful generation of an Ad SV25 based adenoviral vector expressing GFP was confirmed by applying the supernatant from the transfected culture on to fresh cell cultures. The presence of secondarily infected cells was determined by observation of green fluorescence in a population of the cells.

### 2. Construction of E3 Deleted Pan-6 and Pan-7 Vectors.

In order to enhance the cloning capacity of the adenoviral vectors, the E3 region can be deleted because this region encodes genes that are not required for the propagation of the virus in culture. Towards this end, E3-deleted versions of Pan-5, Pan-6, Pan-7, and C68 have been made (a 3.5 kb Nru-AvrII fragment containing E31-9 is deleted).

### E3 Deletion in Pan6 Based Vector

E1-deleted pPan6-pkGFP molecular clone was digested with Sbf I and Not I to isolate 19.3 kb fragment and ligated back at Sbf I site. The resulting construct pPan6-Sbf I-E3 was treated with Eco 47 III and Swa I, generating pPan6-E3. Finally, 21 kb Sbf I fragment from Sbf I digestion of pPan6-pkGFP was subcloned into pPan6-E3 to create pPan6-E3-pkGFP with a 4 kb deletion in E3.

### E3 Deleted Pan7 Vector

The same strategy was used to achieve E3 deletions in Pan 7. First, a 5.8 kb Avr II fragment spanning the E3 region was subcloned pSL-1180, followed by deletion of E3 by Nru I digestion. The resulting plasmids were treated with Spe I and Avr II to obtain 4.4 kb fragments and clone into pPan7-pkGFP at Avr II sites to replace the original E3 containing Avr II fragments, respectively. The final pPan7-E3-pkGFP construct had a 3.5 kb E3-deletion.

A full description of construction of E1, E3 and E4 deletions in these and other Pan Adenovirus serotypes is given in WO03/0046124. Further information is also available in Human Gene Therapy 15:519-530 (WO03/046124).

### Example 61

### Construction of Gag, RT, Nef Sequence

This is described in full in WO03/025003

Plasmid p73i-Tgrn
1. Plasmid: p73i-GRN2 Clone #19 (p17/p24(opt)/RT(opt)trNef)—Repaired

### Gene of Interest:

The p17/p24 portion of the codon optimised Gag, codon optimised RT and truncated Nef gene from the HIV-1 Glade B strain HXB2 downstream of an Iowa length HCMV promoter+exon1, and upstream of a rabbit β-globin poly-adenylation signal.

Plasmids containing the trNef gene derived from plasmid p17/24trNef1 contain a PCR error that gives an R to H amino acid change 19 amino acids from the end of Nef. This was corrected by PCR mutagenesis, the corrected Nef PCR stitched to codon optimised RT from p7077-RT3, and the stitched fragment cut with ApaI and BamHI, and cloned into ApaI/BamHI cut p73i-GRN.

### Primers:

PCR coRT from p7077-RT3 using primers:

(Polymerase=PWO (Roche) throughout.

### Sense: U1

### GAATTCGCGGCCGCGATGGGCCCCATCAGTCCCATCGAGACCGTGCCGGTGAA GCTGAAACCCGGGAT

### AScoRT-Nef

### GGTGTGACTGGAAAACCCACCATCAGCACCTTTCTAATCCCCGC

Cycle: 95° C. (30 s) then 20 cycles 95° C. (30 s), 55° C. (30 s), 72° C. (180 s), then 72° C. (120 s) and hold at 4° C.
The 1.7 kb PCR product was gel purified.
PCR 5′ Nef from p17/24trNef1 using primers:

### Sense: S-Nef

### ATGGTGGGTTTTCCAGTCACACC

### Antisense: ASNef-G:

### GATGAAATGCTAGGCGGCTGTCAAACCTC

Cycle: 95° C. (30 s) then 15 cycles 95° C. (30 s), 55° C. (30 s), 72° C. (60 s), then 72° C. (120 s) and hold at 4° C.
PCR 3′ Nef from p17/24trNef1 using primers:

### Sense: SNEF-G

### GAGGTTTGACAGCCGCCTAGCATTTCATC

### Antisense:

AStrNef (antisense)

### CGCGGATCCTCAGCAGTTCTTGAAGTACTCC

Cycle: 95° C. (30 s) then 15 cycles 95° C. (30 s), 55° C. (30 s), 72° C. (60 s), then 72° C. (120 s) and hold at 4° C.
The PCR products were gel purified. Initially the two Nef products were stitched using the 5′ (S-Nef) and 3′ (AstrNef) primers.
Cycle: 95° C. (30 s) then 15 cycles 95° C. (30 s), 55° C. (30 s), 72° C. (60 s), then 72° C. (180 s) and hold at 4° C.
The PCR product was PCR cleaned, and stitched to the RT product using the U1 and AstrNef primers:
Cycle: 95° C. (30 s) then 20 cycles 95° C. (30 s), 55° C. (30 s), 72° C. (180 s), then 72° C. (180 s) and hold at 4° C.

The 2.1 kb product was gel purified, and cut with ApaI and BamHI. The plasmid p73I-GRN was also cut with Apa1 and BamHI gel purified and ligated with the ApaI-Bam RT3trNef to regenerate the p17/p24(opt)/RT(opt)trNef gene.

Article II. 2. Plasmid: p73I-RT w229k (Inactivated RT)

### Gene of Interest:

Generation of an inactivated RT gene downstream of an Iowa length HCMV promoter+exon 1, and upstream of a rabbit β-globin poly-adenylation signal.

Due to concerns over the use of an active HIV RT species in a therapeutic vaccine inactivation of the gene was desirable. This was achieved by PCR mutagenesis of the RT (derived from P73I-GRN2) amino acid position 229 from Trp to Lys (R7271 p1-28).

### Primers:

PCR 5′ RT+mutation using primers:
(polymerase=PWO (Roche) throughout)

### Sense: RT3-u:1

### GAATTCGCGGCCGCGATGGGCCCCATCAGTCCCATCGAGACCGTGCCGGTGAA GCTGAAACCCGGGAT

### Antisense: AScoRT-Trp229Lys

### GGAGCTCGTAGCCCATCTTCAGGAATGGCGGCTCCTTCT

### Cycle:

1×[94° C. (30 s)]
15×[94° C. (30 s)/55° C. (30 s)/72° C. (60 s)]
1×[72° C. (180 s)]
PCR gel purify
PCR 3′ RT+mutation using primers:

### Antisense: RT3-1:1

### GAATTCGGATCCTTACAGCACCTTTCTAATCCCCGCACTCACCAGCTTGTCGAC CTGCTCGTTGCCGC

### Sense: ScoRT-Trp229Lys

### CCTGAAGATGGGCTACGAGCTCCATG

### Cycle:

1×[94° C. (30 s)]
15×[94° C. (30 s)/55° C. (30 s)/72° C. (60 s)]
1×[72° C. (180 s)]
PCR gel purify

The PCR products were gel purified and the 5′ and 3′ ends of RT were stitched using the 5′ (RT3-U1) and 3′ (RT3-L1) primers.

### Cycle:

1×[94° C. (30 s)]
15×[94° C. (30 s)/55° C. (30 s)/72° C. (120 s)]
1×[72° C. (180 s)]

The PCR product was gel purified, and cloned into p7313ie, utilising NotI and BamHI restriction sites, to generate p73I-RT w229k.

Article III. 3. Plasmid: p73i-Tgrn

### Gene of Interest:

The p17/p24 portion of the codon optimised gag, codon optimised RT and truncated Nef gene from the HIV-1 Glade B strain HXB2 downstream of an Iowa length HCMV promoter+exon1, and upstream of a rabbit β-globin poly-adenylation signal.

Triple fusion constructs which contain an active form of RT, may not be acceptable to regulatory authorities for human use thus inactivation of RT was achieved by Insertion of a NheI and ApaI cut fragment from p73i-RT w229k, into NheI/ApaI cut p73i-GRN2#19. This results in a W→K change at position 229 in RT.

The full sequence of the Tgrn plasmid insert is shown in FIG. 51. This contains p17 p24 (opt) Gag, p66 RT (opt and inactivated) and truncated Nef. A map of plasmid p73i-Tgrn is shown in FIG. 52.

### Example 62

### Insertion of Gag, RT, Nef Sequence into Adenovirus

Subcloning of GRN Expression Cassette into pShuttle Plasmid.

The entire expression cassette consisting of promoter, cDNA and polyadenylation signal was isolated from pT-GRN constructs by Sph I and EcoR I double digestion. The Sph I end of the Sph I/EcoR I fragment was filled in with Klenow and cloned into pShuttle plasmid at EcoR I and Mlu I sites where the Mlu I end was blunted.

During the cloning process an additional flanking sequence became associated with the HIV expression cassette. This sequence is known as the Cer sequence and has no known function.

Transfer of GRN Expression Cassette into E1/E3-Deleted Molecular Clones of Pan6 and Pan7 Vectors.

The expression cassette was retrieved from pShuttle by I-Ceu I and PI-Sce I digestions and cloned into the same sites of the molecular clones of Pan6 and Pan7 vectors. Recombinant clones were identified through green/white selection and confirmed by extensive restriction enzyme analysis.

### Rescue and Propagation of Recombinant Viruses.

Molecular clones of C6 and C7 vectors were treated with appropriate restriction endonucleases (PmeI and PacI respectively) to release intact linear vector genomes and transfected into 293 cells using the calcium phosphate method. When full cytopathetic effect was observed in the transfected cells, crude viral lysate was harvested and gradually expanded to large scale infections in 293 cells (1×10e9 cells). Viruses from large scale infections were purified by standard CsCl sedimentation method.

In addition the pShuttle plasmid can be further trimmed by cutting with EcoRI and XmnI to remove a 3′ linker sequence and reduce the plasmid size to produce pShuttleGRNc.

### Abbreviations

DOPE=1,2-dioleoyl-syn-glycero-3-phospho-ethanolamine
DMRIE-C=1,2-dimyristyloxypropyl-3-dimethyl-hydroxy ethyl ammonium bromide and cholesterol, (1;1, M/M mix)
jet-PEI=linear polyethyleneimine
jet-PEI-Man=mannose-conjugated linear polyethyleneimine
EBSS=Earle's balanced salt solution
PBS=Phosphate buffered saline
TBS=Tris buffered saline
HEPES=N-(2-Hydroxyethyl)piperazine-N′-(2-ethanesulfonic acid)

### QELS=Quasi-Elastic Light Scattering

### ELS=Electrophoretic Light Scattering

NHP=Non-human primate
p.f.u.=Plaque forming unit

### Ad=Adenovirus

PMED=Particle mediated epidermal delivery (Gene Gun)

### FIGURES

FIG. 1: Luciferase activity in mouse skin 24 hours post DNA delivery

FIG. 2: Luciferase activity in mouse skin 24 hours post DNA delivery

FIG. 3: Luciferase activity in mouse skin 24 hours post DNA delivery

FIG. 4: Immunisation schedule

FIG. 5: Interferon γ ELISPOT Day 8 post-boost

FIG. 6: Interleukin-2 ELISPOT Day 8 post-boost

FIG. 7: Interferon γ ELISPOT Day 14 post-boost

FIG. 8: Interleukin-2 ELISPOT Day 14 post-boost

FIG. 9: Interferon γ ELISPOT Day 21 post-boost

FIG. 10: Interleukin-2 ELISPOT Day 21 post-boost

FIG. 11: Humoral response: whole IgG ELISA on sera samples day 14 post-boost

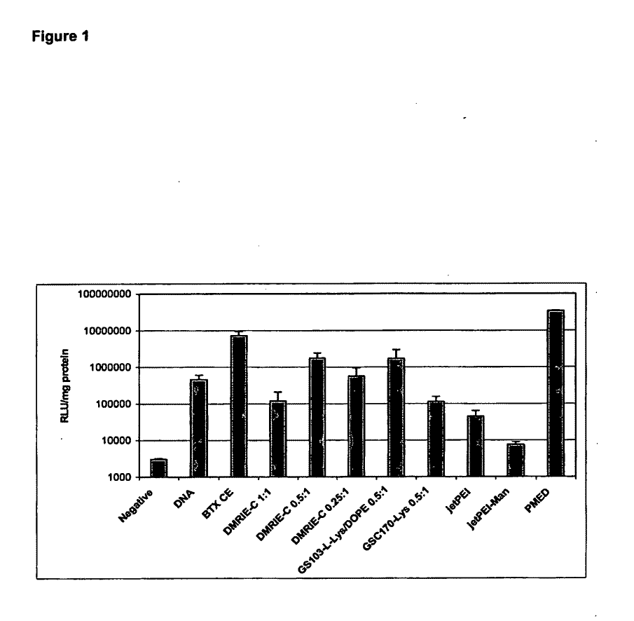

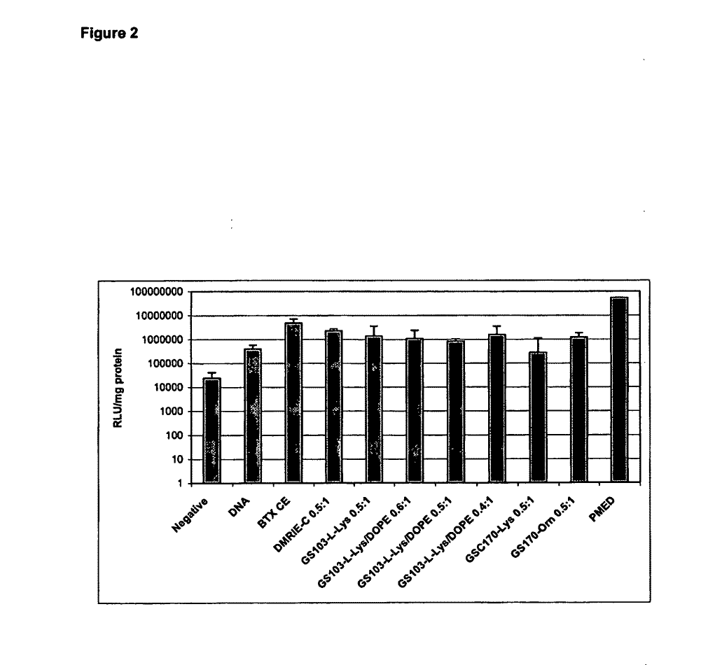

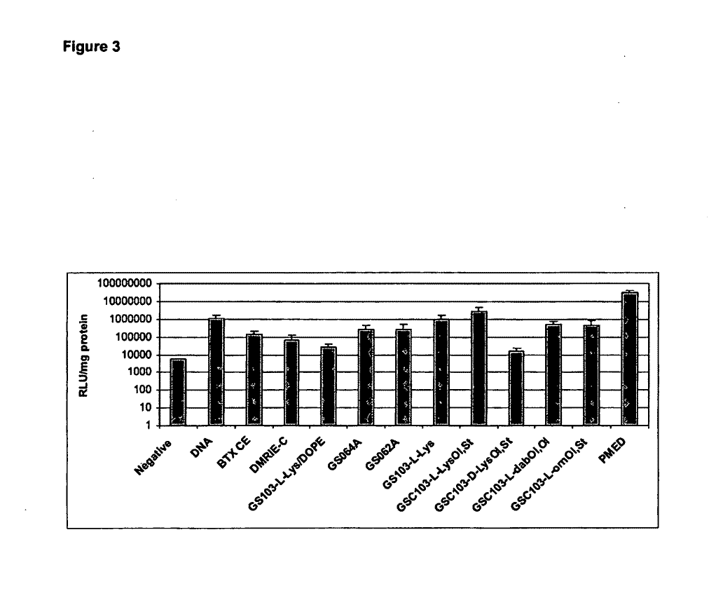

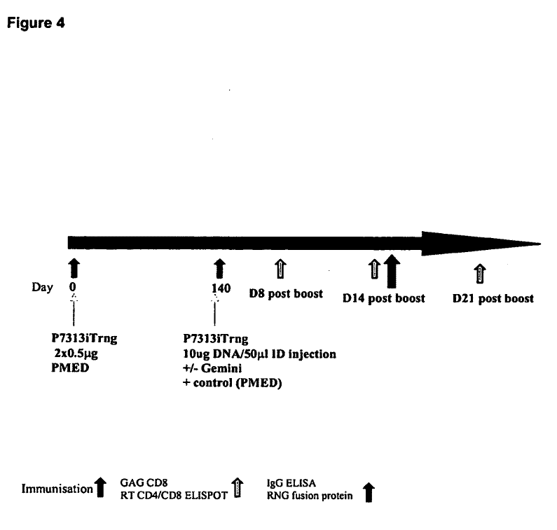

### Key to FIGS. 1-3

Luciferase activity is shown as RLU/mg protein (relative light units per mg protein).

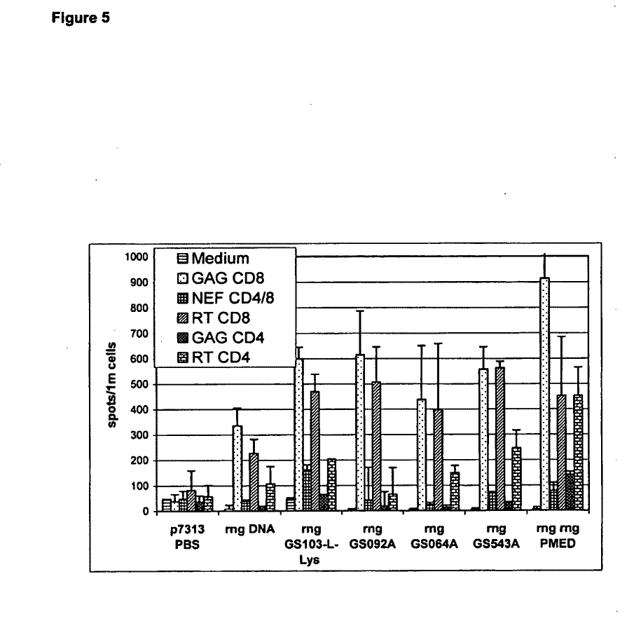

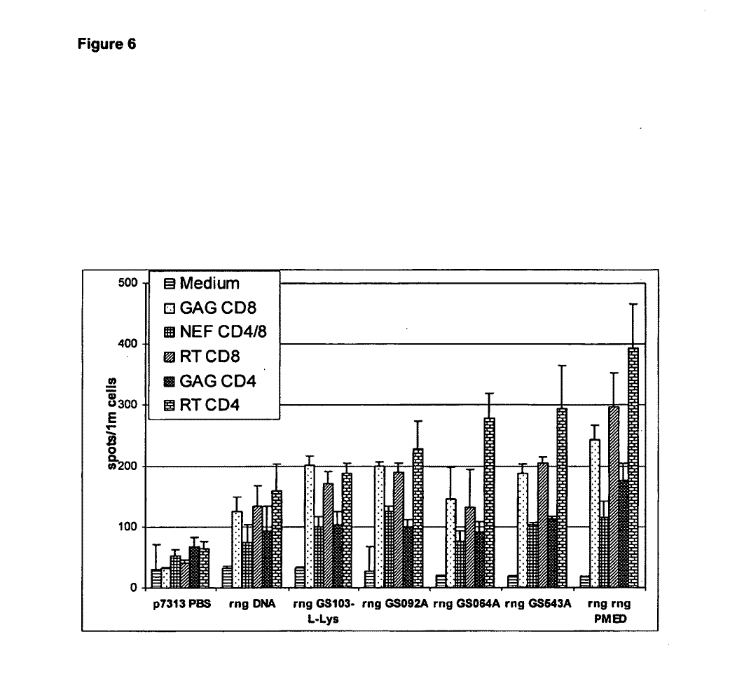

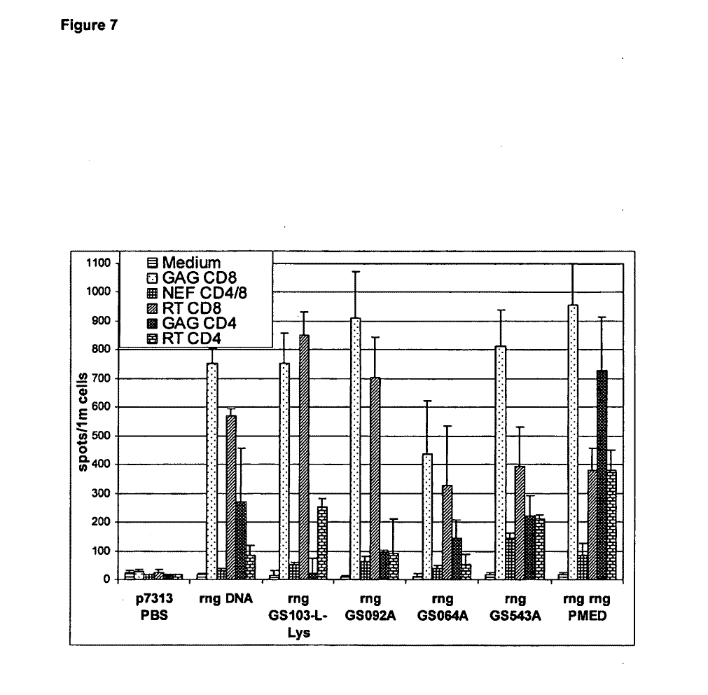

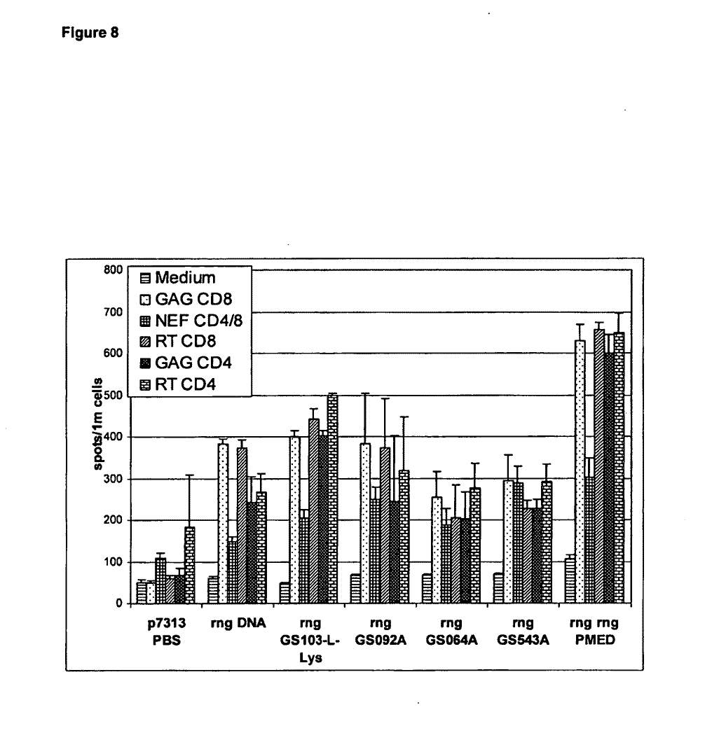

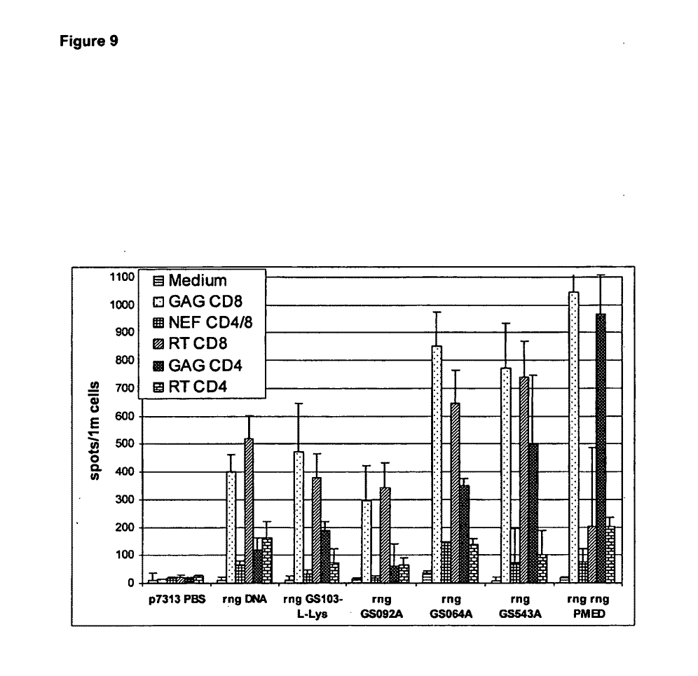

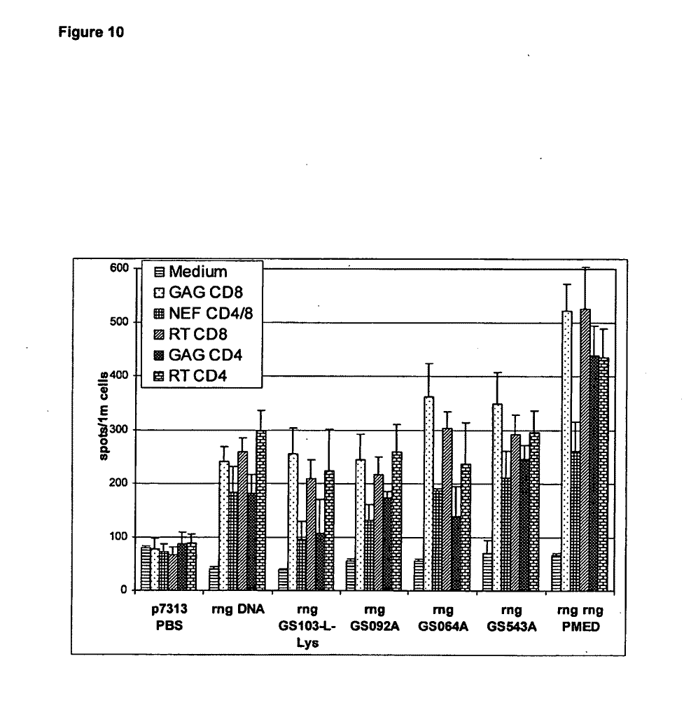

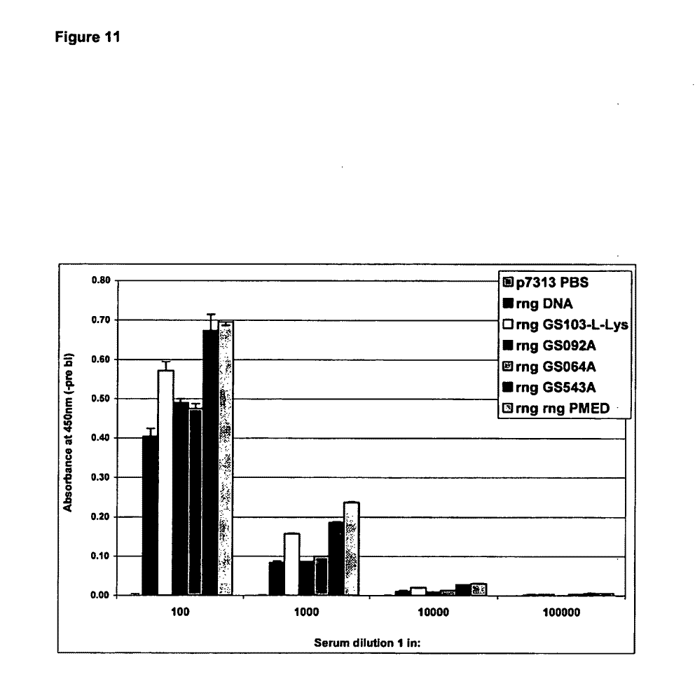

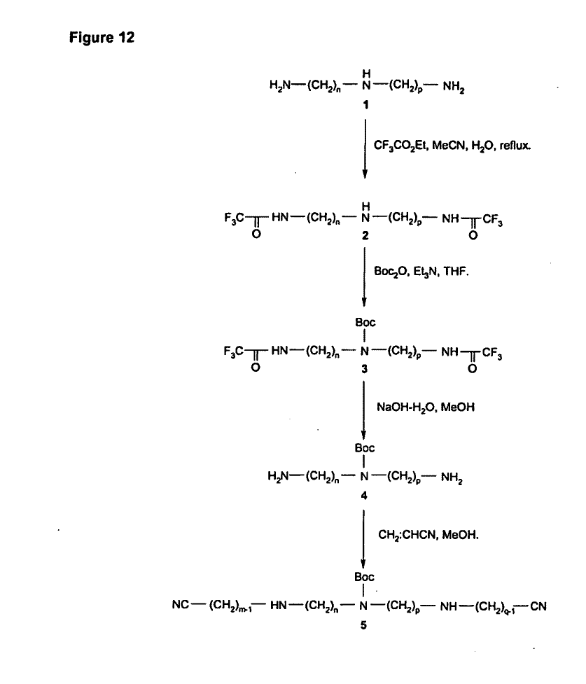

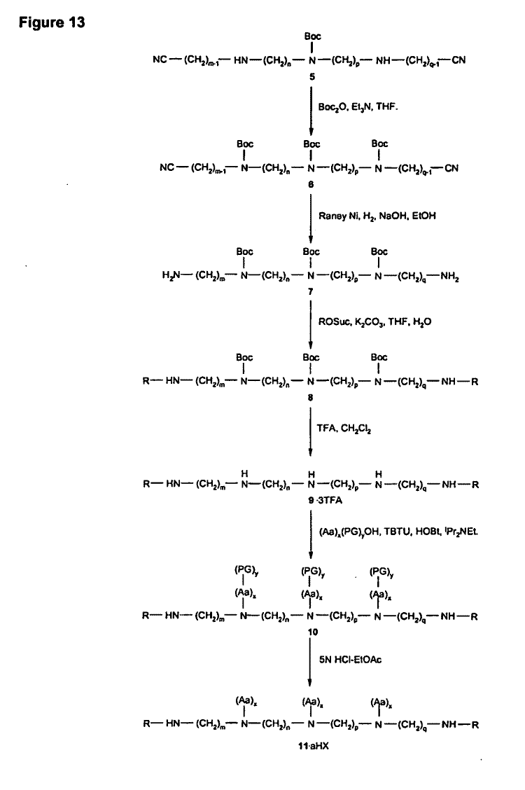

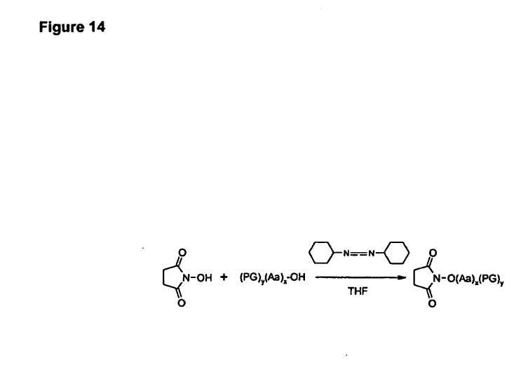

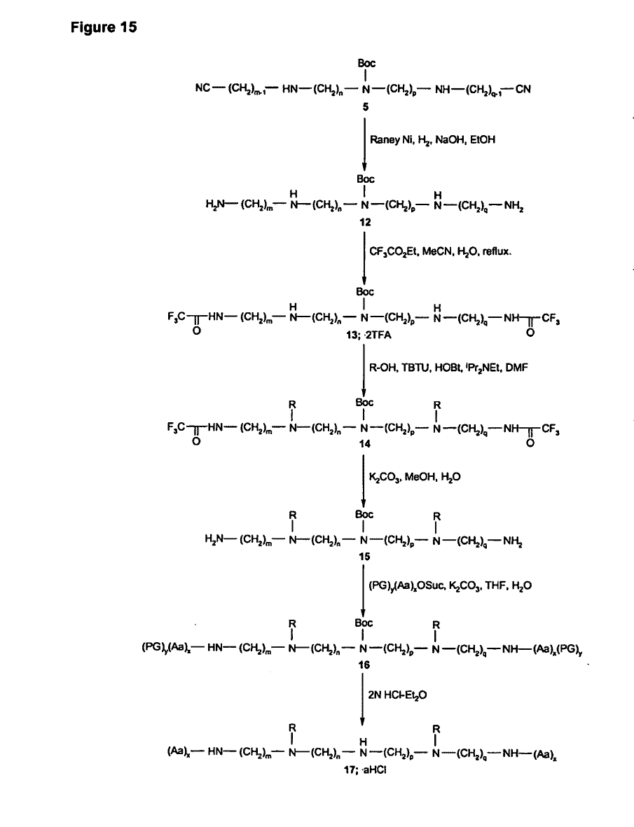

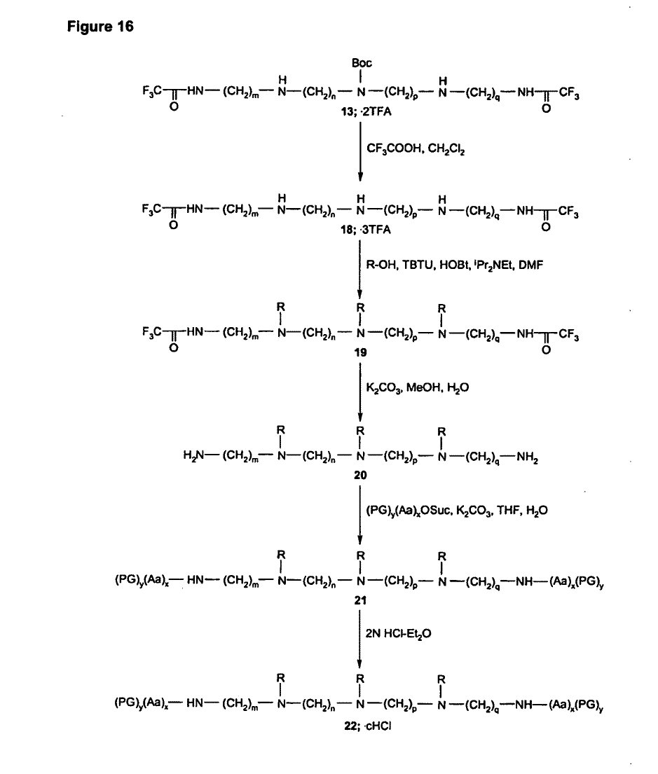

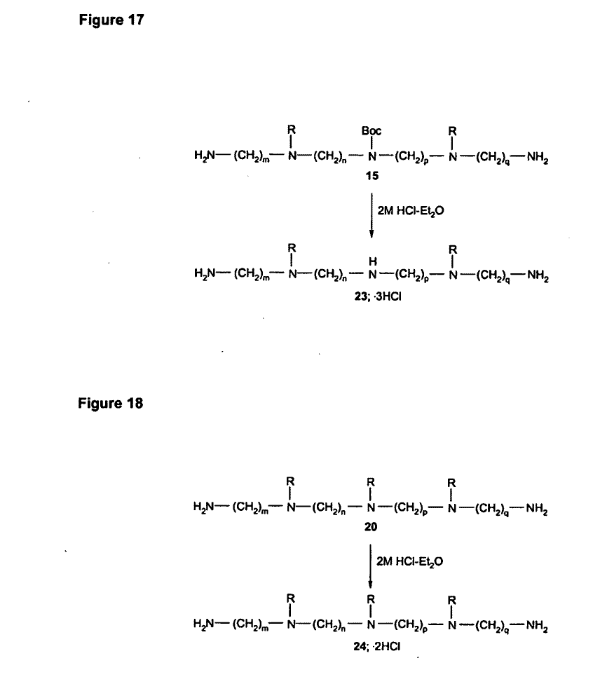

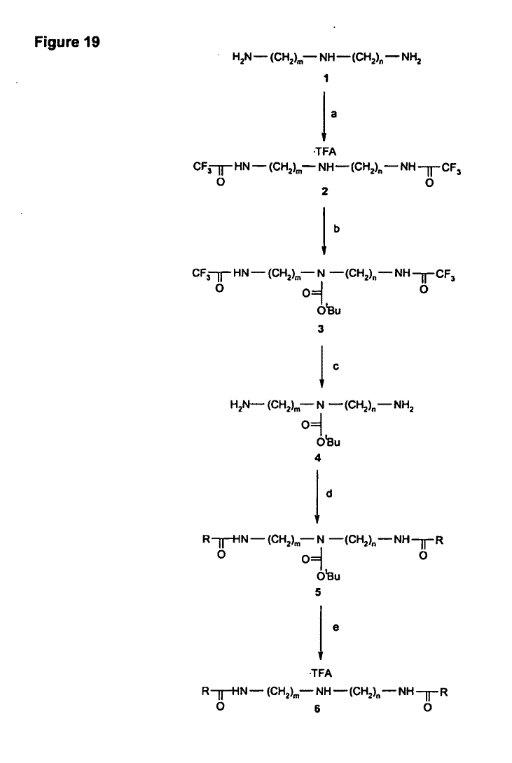

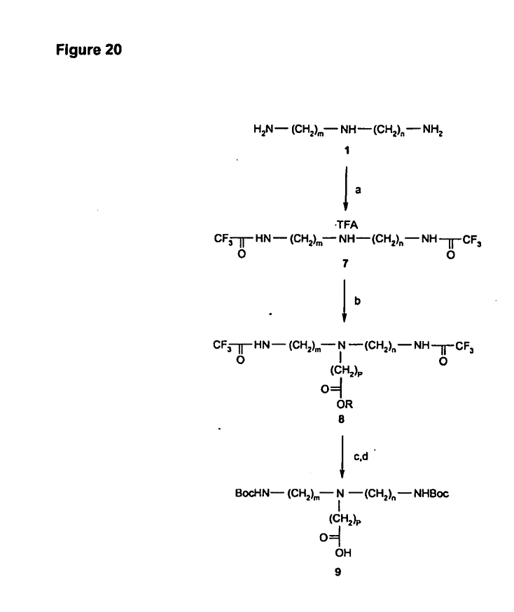

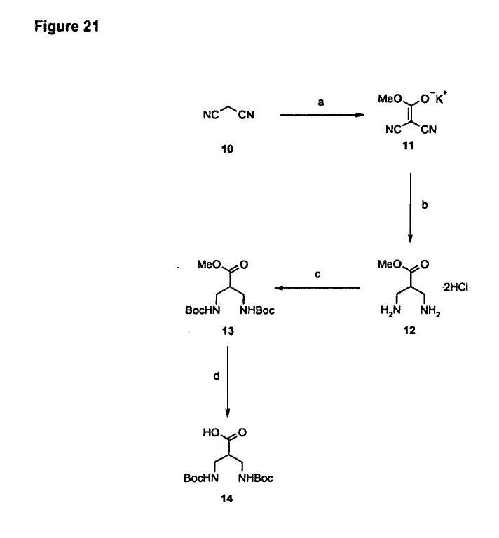

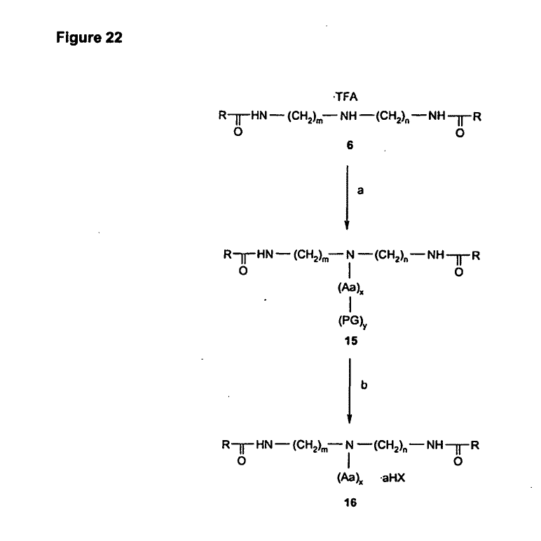

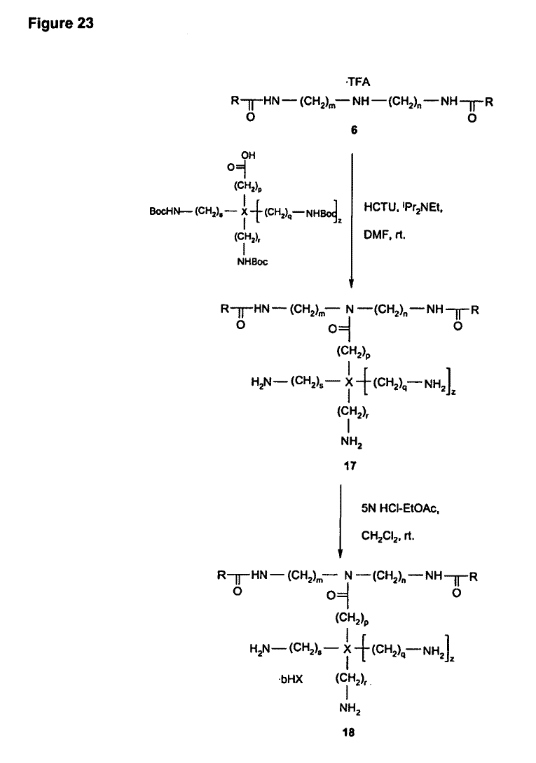

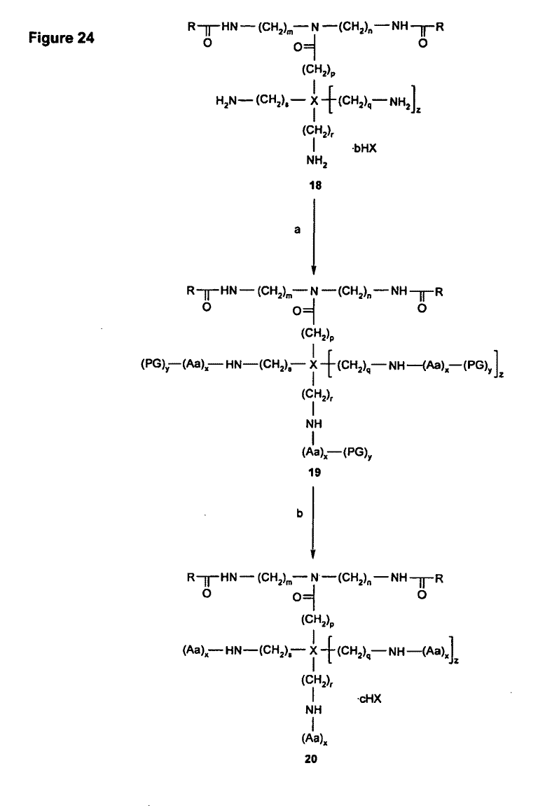

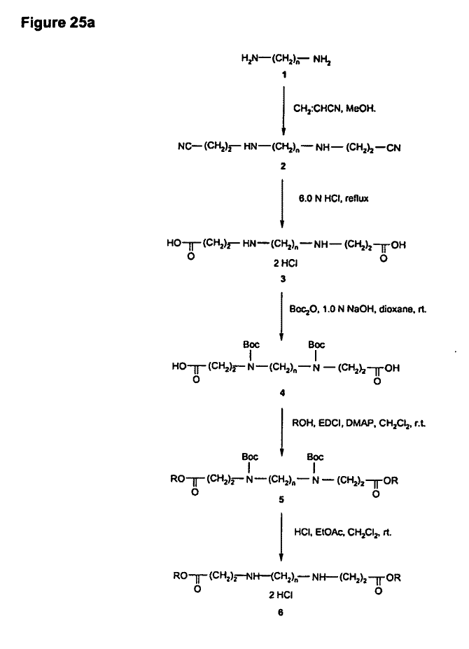

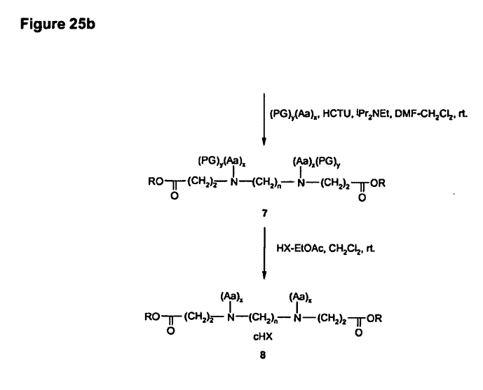

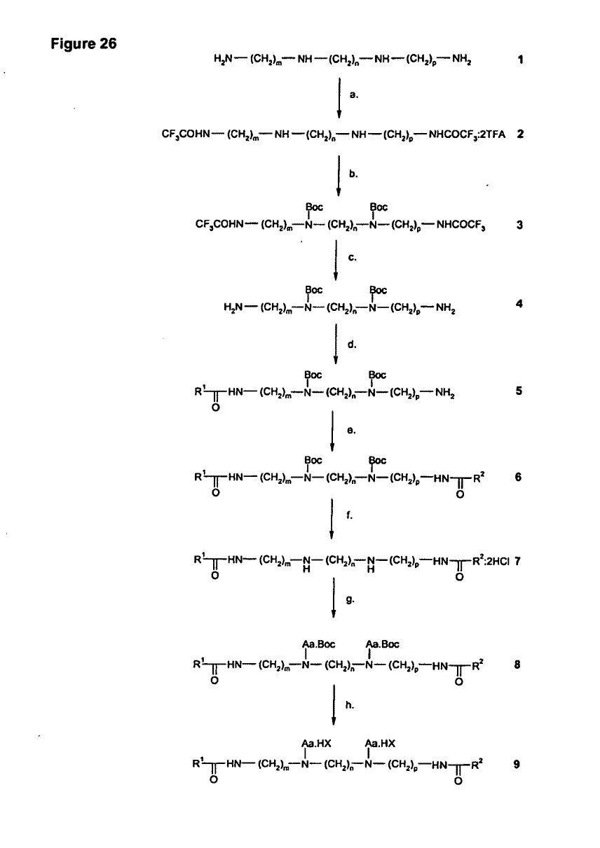

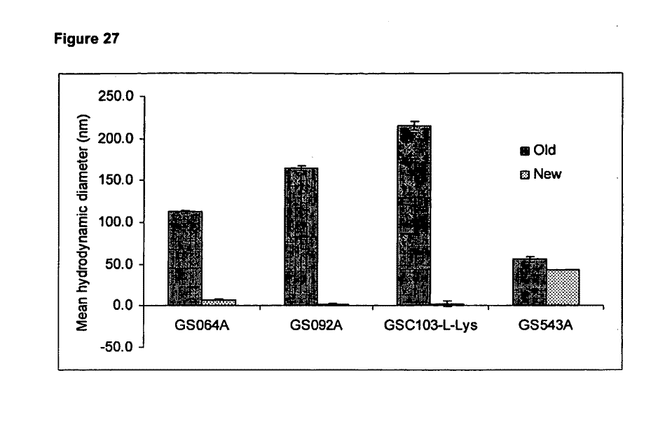

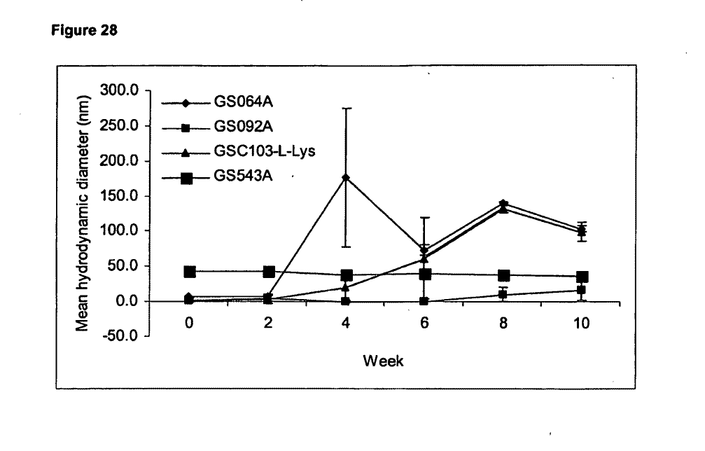

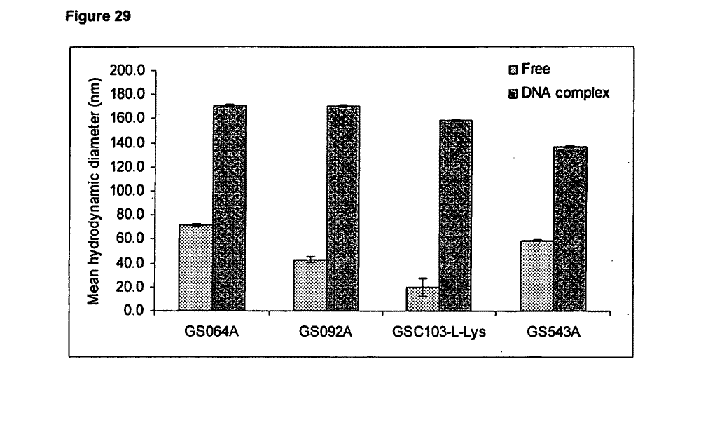

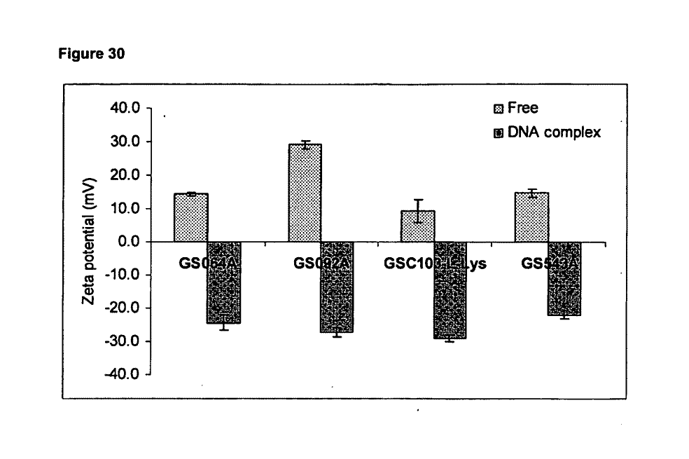

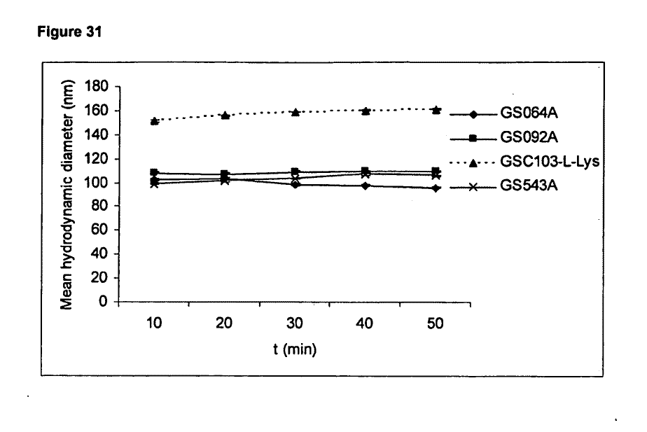

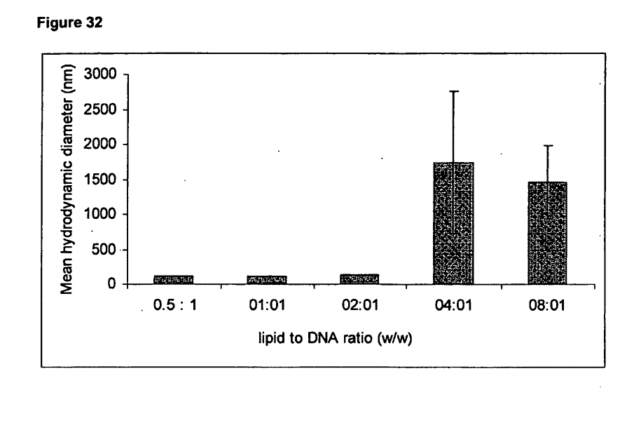

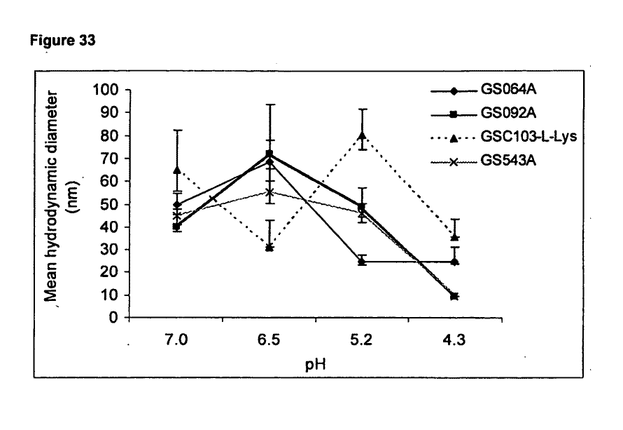

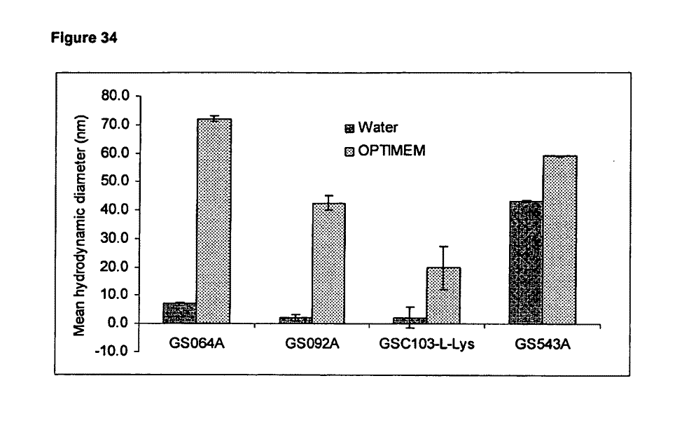

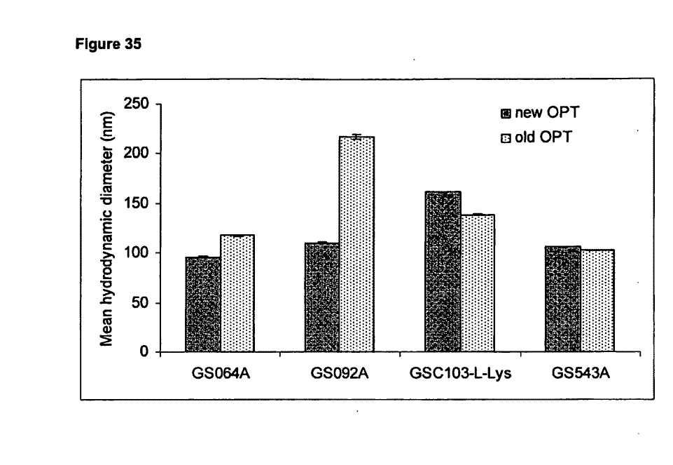

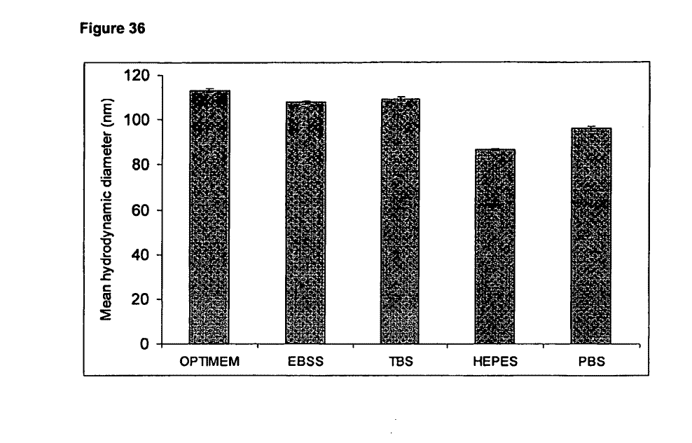

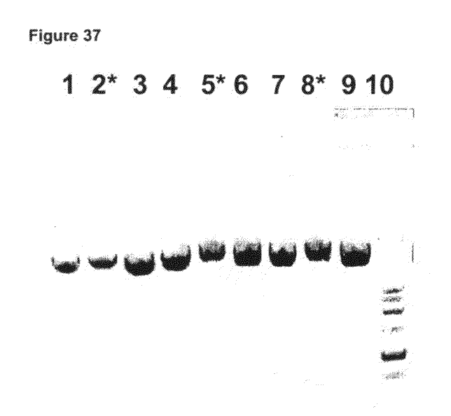

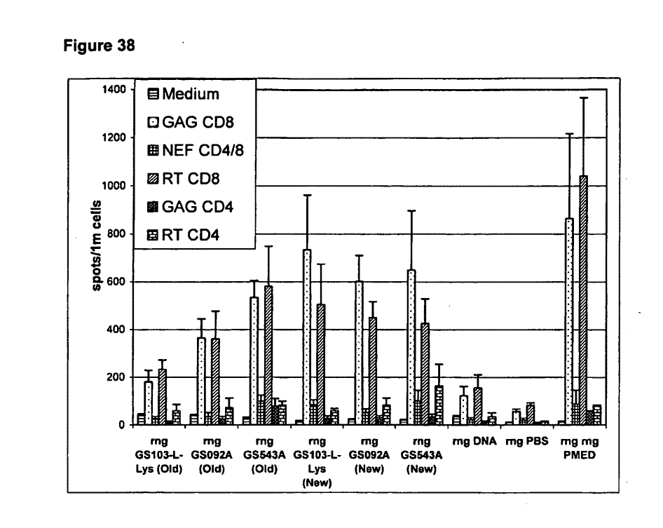

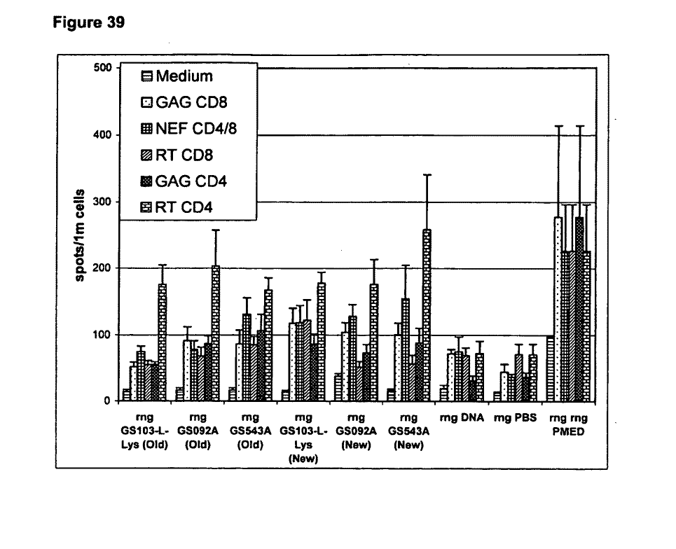

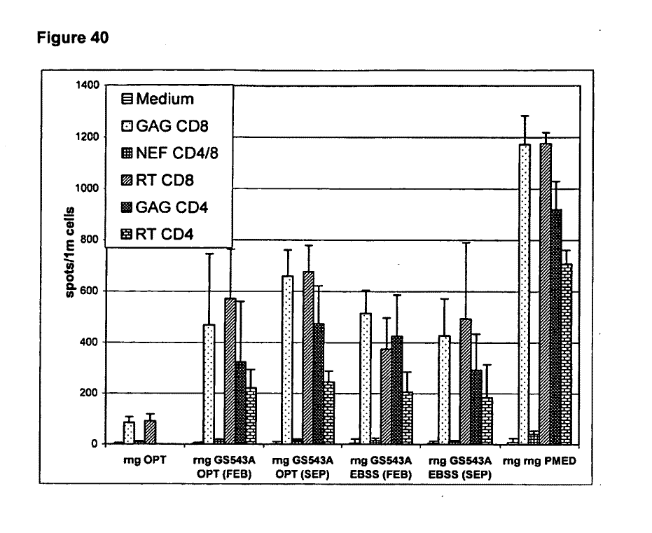

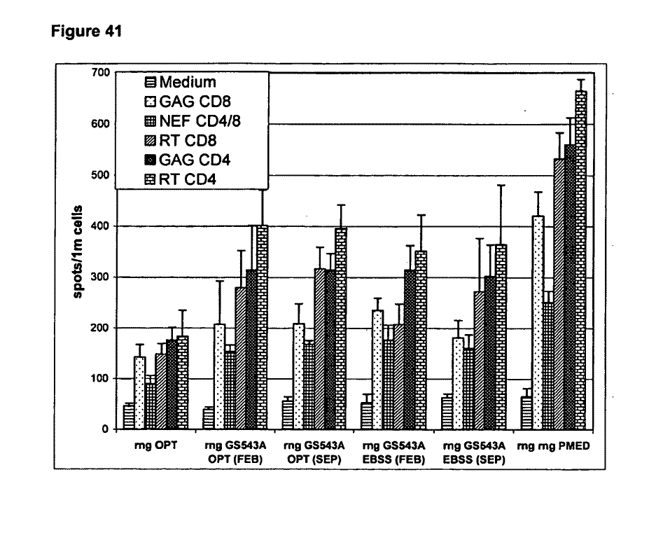

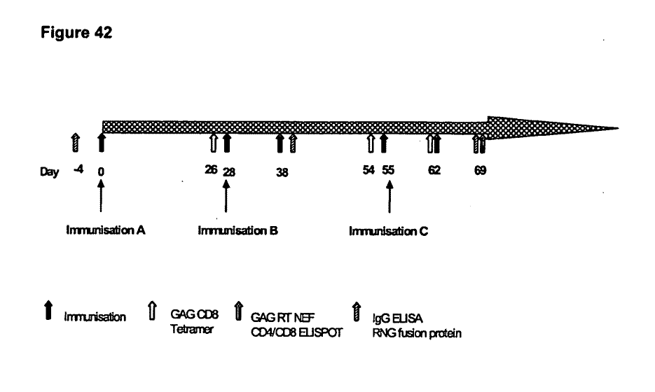

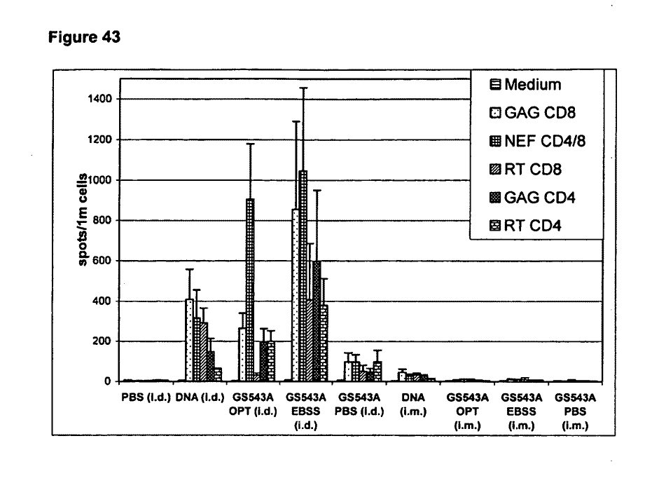

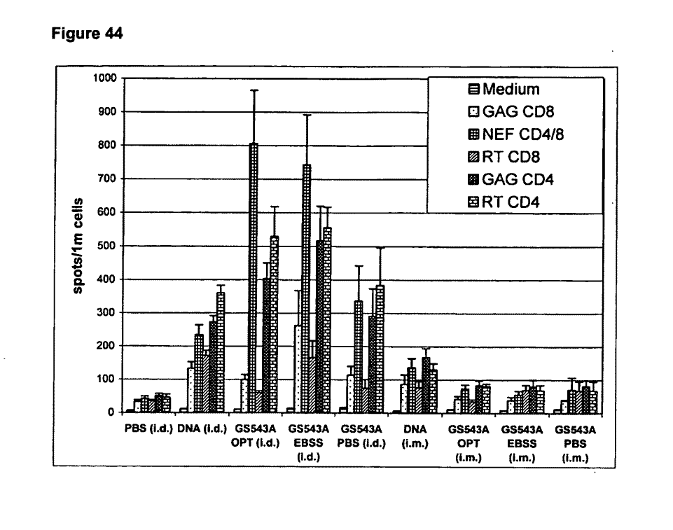

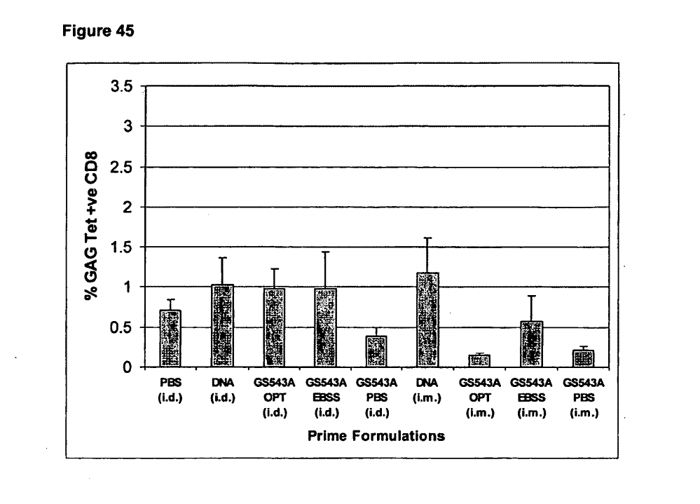

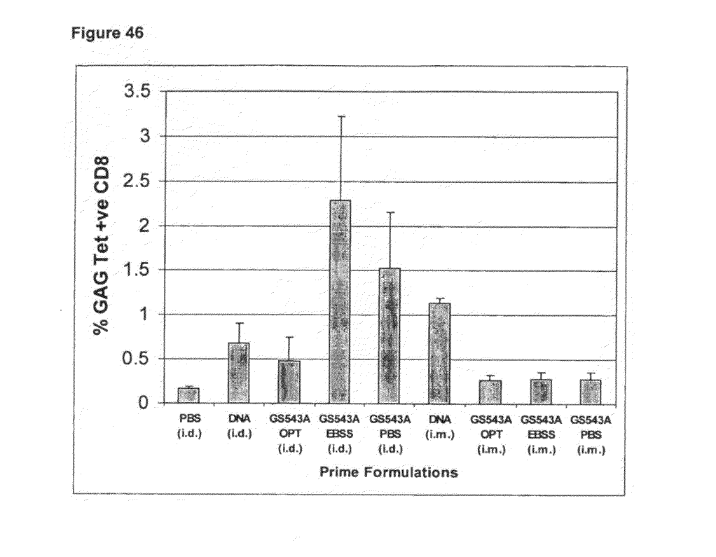

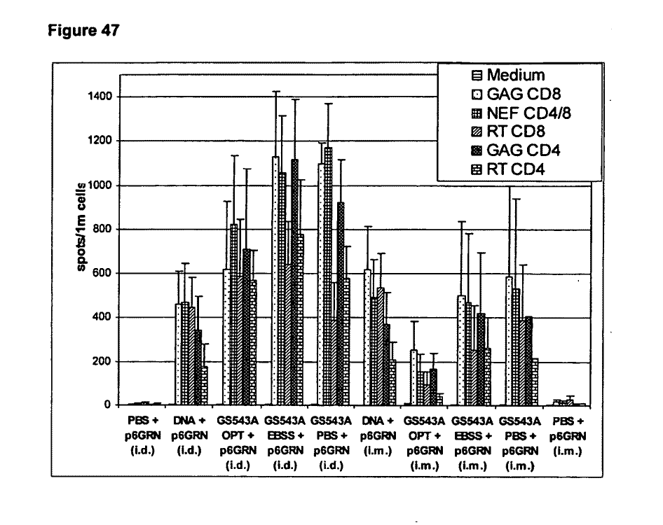

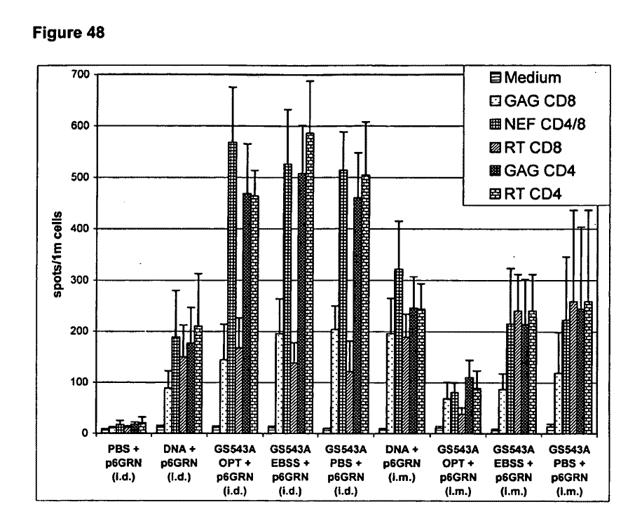

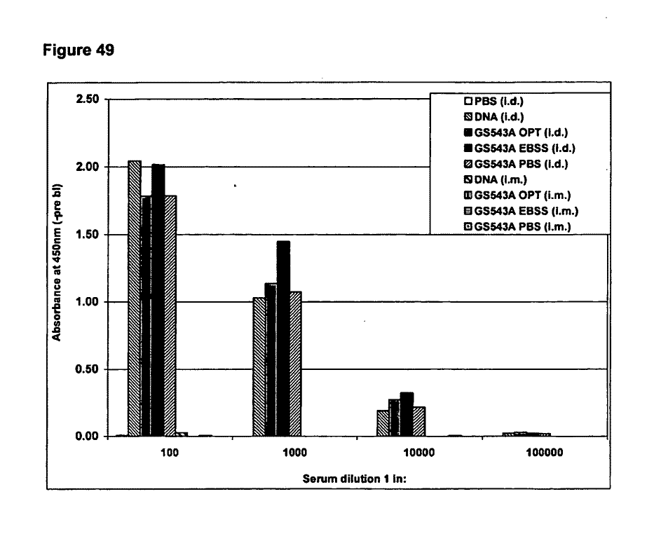

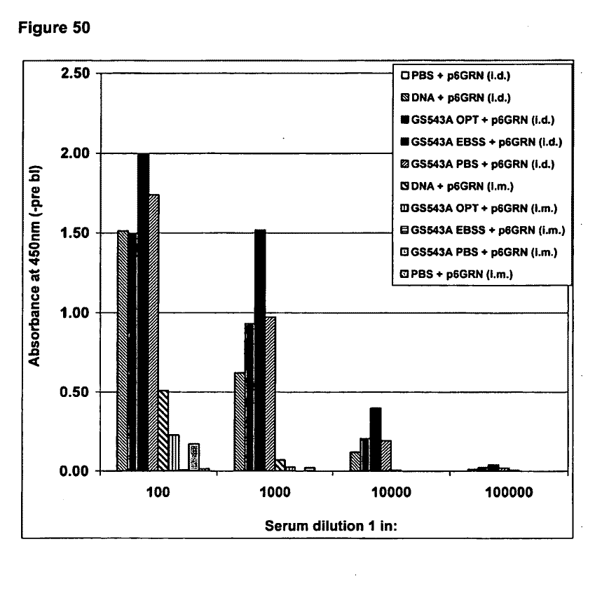

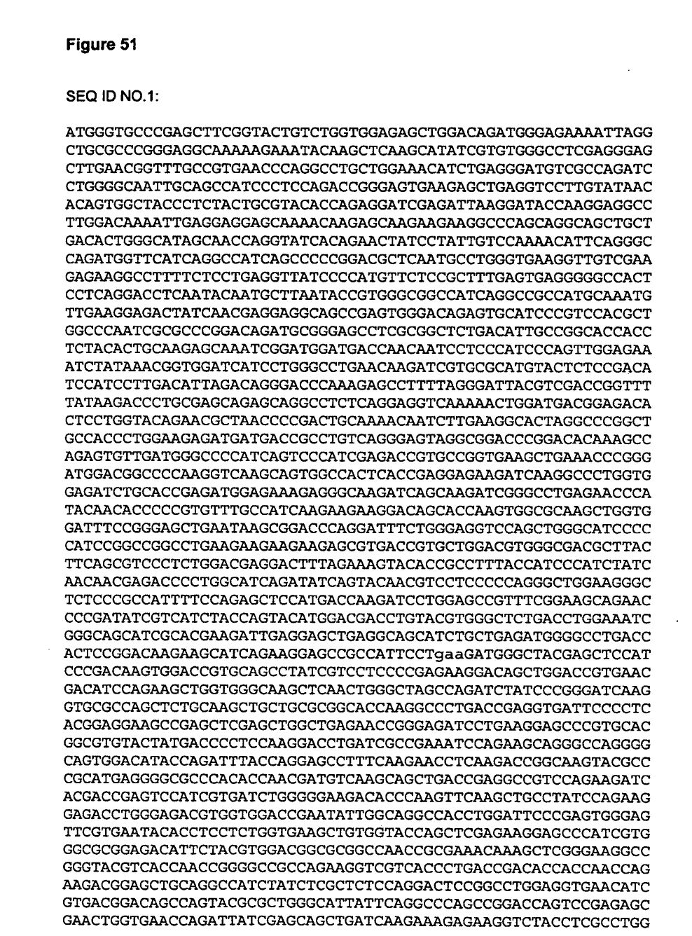

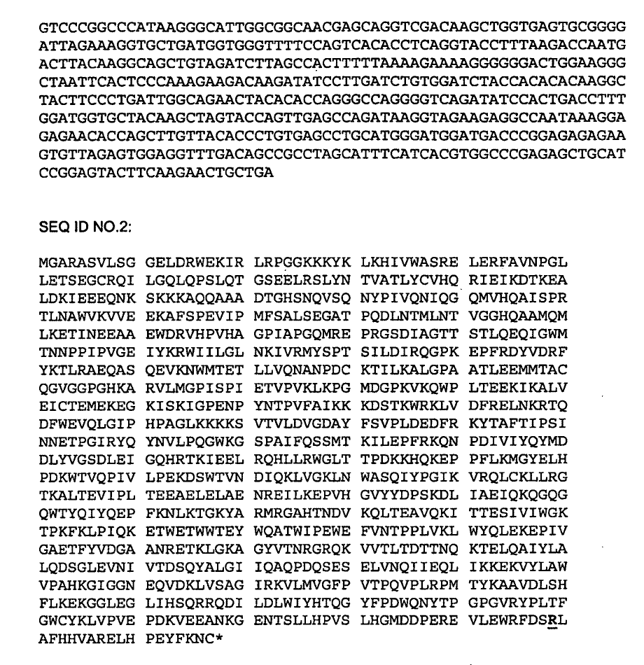

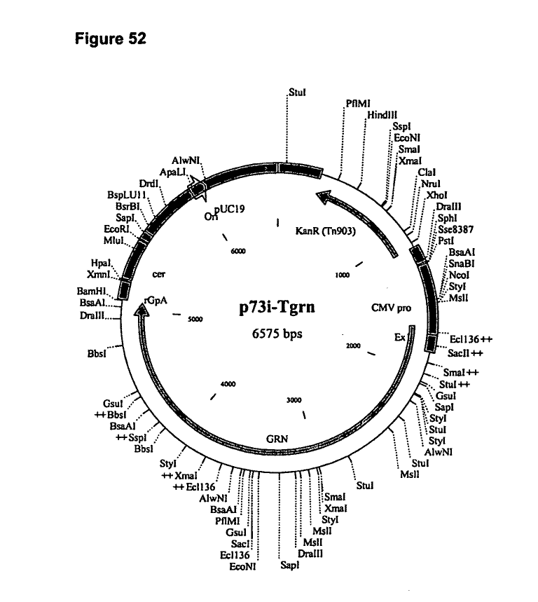

### Key to FIGS. 5-11

Prime: p7313=p7313ie (empty vector), mg=p7313ieTrng
Boost: PBS=mock boost, DNA=naked DNA in 2×PBS, GS=Gemini Surfactant+DNA, mg PMED=PMED boost

FIG. 12: shows a general scheme for the synthesis of an advanced intermediate 5 useful in the synthesis of pentamine gemini compounds.

FIG. 13: shows a general scheme for the synthesis of pentamine gemini compounds.

FIG. 14: shows a reaction scheme for the preparation of an activated amino acid (Aa)ₓ group useful in the synthesis of pentamine gemini compounds.

FIG. 15: shows a general scheme for the synthesis of pentamine gemini compound.

FIG. 16: shows a general scheme for the synthesis of pentamine gemini compounds.

FIG. 17: shows a general reaction scheme for the deprotection of an advanced intermediate for the generation of a salt of a pentamine gemini compound.

FIG. 18: shows a reaction scheme for the generation of a salt of a pentamine Gemini compound.

FIG. 19: shows a general scheme for the synthesis of a protected (Aa) group 6 useful in the synthesis of spermidine-based gemini compounds.

Reagents & conditions: a) CF₃CO₂Et, H₂O, MeCN, reflux; b) Boc₂O, ⁱPr₂NEt, THF, rt; c) NaOH—H₂O, MeOH, 10° C.—rt; d) RCO₂NSuc, K₂CO₃, THF, H₂O, rt; e) CF₃CO₂H, CH₂Cl₂, rt.

FIG. 20: shows a general scheme for the synthesis of a protected (Aa) group 9 useful in the synthesis of spermidine-based gemini compounds.

Reagents & conditions: a) CF₃CO₂Et, H₂O, MeCN, reflux; b) Br(CH₂)ₚCO₂R, ⁱPr₂NEt, MeCN, rt; c) NaOH—H₂O, MeOH, rt; d) Boc₂O, H₂O, MeOH, rt.

FIG. 21: shows a general scheme for the synthesis of an advanced intermediate 14 useful in the synthesis of spermidine-based gemini compounds.

Reagents & conditions: a) MeOCOCl, NaOH—H₂O, THF, rt; b) 10% Pd/C, ^{c}HCl, MeOH, H₂, 5 bar; c) Boc₂O, ⁱPr₂NEt, DMF; d) 2N KOH—H₂O, THF, rt.

FIG. 22: shows a general scheme for the synthesis of spermidine-based molecules.

Reagents & conditions: a) (PG)_{y}(Aa)ₓ, HCTU, ⁱPrNEt₂, DMF, rt; b) 5N HCl-EtOAC, CH₂Cl₂, rt.

FIG. 23: shows a general scheme for the synthesis of spermidine-based molecules.

FIG. 24: shows a general scheme for the synthesis of spermidine-based molecules.

Reagents & conditions: a) (PG)_{y}(Aa)ₓ, HCTU or HBTU, ⁱPr₂NEt, DMF, rt.; b) 5N HCl-EtOAc, CH₂Cl₂, rt.

FIG. 25: shows a general scheme for the synthesis of an ester-linked surfactant.

FIG. 26: shows a general scheme for the synthesis of unsymmetrical sperimine-based Gemini compounds. Reagents & conditions: (a). CF₃CO₂Et, MeCN, reflux, 18 h; (b). (Boc)₂O, ⁱPr₂NEt, THF; (c). K₂CO₃, H₂O, MeOH, reflux, 2 h; (d). R¹CO₂C₆F₅, Et₃N, CH₂Cl₂, −78° C.—rt; (e). R²CO₂H, TBTU, HOBt, ⁱPr₂NEt, CH₂Cl₂; (f) HCl, Et₂O; (g). Aa.Boc, TBTU, HOBt, ⁱPr₂NEt, CH₂Cl₂; (h) HCl, Et₂O, rt.

FIG. 27: Analysis of gemini surfactants, (‘old’ and ‘new’ stocks) by QELS.

FIG. 28: Analysis of gemini surfactants by QELS: Time course for freshly prepared stocks.

FIG. 29: Analysis of gemini surfactants by QELS: Complex formation with plasmid DNA

FIG. 30: Analysis of gemini surfactants by ELS: Complex formation with plasmid DNA

FIG. 31: Analysis of gemini surfactants by QELS: Time course after complex formation with plasmid DNA

FIG. 32: Analysis of GS543A by QELS: Effect of varying GS543A to DNA ratio

FIG. 33: Analysis of gemini surfactants by QELS: Investigation of the pH sensitivity of Gemini Surfactant-DNA complexes

FIG. 34: Analysis of gemini surfactants formed in water or diluted in OPTIMEM by QELS

FIG. 35: Analysis of gemini surfactants by QELS: Gemini Surfactant-DNA complexes formed in fresh (‘new’) or stored (‘old’) OPTIMEM

FIG. 36: Analysis of gemini surfactants by QELS: Gemini Surfactant-DNA complexes formed in different buffers

FIG. 37: Analysis of GS543A and DNA lipoplexes in Optimem (OPT), EBSS or PBS buffers by agarose gel retardation.

FIG. 38: Cellular responses following i.d. DNA immunisation with gemini surfactants, (‘old’ and ‘new’ stocks), in PMED primed Balb/c mice: Interferon γ ELISPOT Day 14 post-boost

FIG. 39: Cellular responses following i.d. DNA immunisation with gemini surfactants, (‘old’ and ‘new’ stocks), in PMED primed Balb/c mice: Interleukin-2 ELISPOT Day 14 post-boost

FIG. 40: Cellular responses following i.d. immunisation with GS543A: DNA complexes, (different stocks buffered by OPTIMEM or EBSS), in PMED primed Balb/c mice: Interferon γ ELISPOT Day 14 post-boost

FIG. 41: Cellular responses following i.d. immunisation with GS543A: DNA complexes, (different stocks buffered by OPTIMEM or EBSS), in PMED primed Balb/c mice: Interleukin-2 ELISPOT Day 14 post-boost

FIG. 42: Immunisation schedule for analysis of effect of GS543A on responses to DNA immunisation in homologous regimes followed by a heterologous boost with Adenovirus via the i.d. and i.m. routes

FIG. 43: Cellular responses following i.d. or i.m. immunisations with GS543A: DNA complexes, (buffered by OPTIMEM or EBSS or PBS), in Balb/c mice: Interferon γ ELISPOT Day 10 post-boost, (Immunisation B).

FIG. 44: Cellular responses following i.d. or i.m. immunisations with GS543A: DNA complexes, (buffered by OPTIMEM or EBSS or PBS), in Balb/c mice: Interleukin-2 ELISPOT Day 10 post-boost, (Immunisation B).

FIG. 45: Cellular responses following i.d. or i.m. immunisations with GS543A: DNA complexes, (buffered by OPTIMEM or EBSS or PBS), in Balb/c mice: Gag CD8 Tetramer responses Day 26 post-primary immunisation, (A).

FIG. 46: Cellular responses following i.d. or i.m. immunisations with GS543A: DNA complexes, (buffered by OPTIMEM or EBSS or PBS), in Balb/c mice: Gag CD8 Tetramer responses Day 26 post 1^{st} boost immunisation, (B).

FIG. 47: Cellular responses following i.d. or i.m. immunisations with GS543A: DNA complexes, (buffered by OPTIMEM or EBSS or PBS), in Balb/c mice after Adenovirus boost: Interferon γ ELISPOT Day 7 post 2^{nd} heterologous boost, (Immunisation C).

FIG. 48: Cellular responses following i.d. or i.m. immunisations with GS543A: DNA complexes, (buffered by OPTIMEM or EBSS or PBS), in Balb/c mice after Adenovirus boost: Interleukin-2 ELISPOT Day 7 post 2^{nd} heterologous boost, (Immunisation C).

FIG. 49: Humoral responses following i.d. or i.m. immunisations with GS543A: DNA complexes, (buffered by OPTIMEM or EBSS or PBS), in Balb/c mice: whole IgG ELISA on sera samples Day 10 post-boost, (Immunisation B).

FIG. 50: Humoral responses following i.d. or i.m. immunisations with GS543A: DNA complexes, (buffered by OPTIMEM or EBSS or PBS), in Balb/c mice: after Adenovirus boost: whole IgG ELISA on sera samples Day 14 post 2^{nd} heterologous boost, (Immunisation C).

### Sequences (FIG. 51)

| Amino acid or polynucleotide | Sequence Identifier |
|---|---|
| description | (SEQ ID No) |
| Tgrn polynucleotide | 1 |
| Tgrn amino acid | 2 |

FIG. 52: Map of plasmid p73i-Tgrn.

### Key to Figures

### FIG. 27

Old=Gemini Surfactant stored at 4° C. for 4 months
New=Freshly prepared Gemini Surfactants

### FIGS. 29 & 30

Free=Gemini Surfactant in absence of DNA
DNA complex=Gemini Surfactant complexed with plasmid DNA

### FIG. 35

New OPT=Freshly opened OPTIMEM
Old OPT=OPTIMEM opened for 3 months

### FIG. 37

(1) 1 ug plasmid in OPT, (2) 1 ug plasmid+GS543A in OPT*, (3) 2 ug plasmid+GS543A in OPT, (4) 1 ug plasmid in EBSS, (5) 1 ug plasmid+GS543A in EBSS*, (6) 2 ug plasmid+GS543A in EBSS, (7) 1 ug plasmid in PBS, (8) 1 ug plasmid+GS543A in PBS*, (9) 2 ug plasmid+GS543A in PBS, (10) 1 kb ladder; *=lane with retarded plasmid DNA.

### FIG. 42

Day 38=Day 10 post (1^{st}) boost
Day 62=Day 7 post (2^{nd}) boost
Day 69=Day 14 post (2^{nd}) boost
Immunisation A & B=p7313iTrng, 2×10 ug DNA in 50 ul+/−GS543A by i.d. or i.m. injection
Immunisation C=p6grn, 1×10E8 pfu NHP Adenovirus in 50 ul by i.d. or i.m. injection

What is claimed is:

1. An immunogenic composition comprising a polynucleotide encoding an antigen capable of eliciting an immune response and an adjuvant comprising an immunostimulatory quantity of a gemini surfactant, or a derivative thereof.

2. An immunogenic composition according to claim 1 wherein the adjuvant is a gemini surfactant selected from those comprising two hydrocarbyl chains linked to a spermine-based hydrophilic head group; comprising two hydrocarbyl chains linked to a spermidine-based hydrophilic head group; those comprising two or three hydrocarbyl chains linked to a pentamine-based hydrophilic head group, and those comprising two hydrocarbyl chains linked by ester linkage to a hydrophilic headgroup.

3. An immunogenic composition according to claim 1 wherein the adjuvant is a gemini surfactant comprising two hydrocarbyl chains linked to a hydrophilic headgroup.

4. An immunogenic composition according to claim 1 wherein the adjuvant is a gemini surfactant comprising two hydrocarbyl chains linked to a hydrophilic headgroup by an ester linkage.

5. An immunogenic composition according to claim 1 wherein the adjuvant is a gemini surfactant selected from GS092A and GS543A.

6. An immunogenic composition according to claim 1 wherein the polynucleotide encodes an antigen selected from Nef, Gag, RT, Pol, Env or immunogenic derivatives or fragments thereof.

7. A method of eliciting an immune response in a mammalian subject comprising administering to said individual an immunogenic composition according to claim 1.

8. A vaccine comprising the immunogenic composition as claimed in claim 1.

9. A kit comprising (a) a composition comprising a polynucleotide encoding an antigen, and (b) an adjuvant comprising an immunostimulatory quantity of a gemini surfactant, or a derivative thereof, the kit optionally further comprising instructions to administer the two compositions simultaneously or separately.

10. (canceled)

11. A method of treating an individual infected with HIV comprising administration to that individual of an immunogenic composition as claimed in claim 1.


SEQUENCE LISTING (14 sequences)

SEQ ID NO: 1 - DNA, 3204 bp, Artificial Sequence
  Tgrn plasmid insert
atgggtgcccgagcttcggtactgtctggtggagagctggacagatgggagaaaattaggctgcgcccgggaggcaaaaagaaatacaagctcaagcatatcgtgtgggcctcgagggagcttgaacggtttgccgtgaacccaggcctgctggaaacatctgagggatgtcgccagatcctggggcaattgcagccatccctccagaccgggagtgaagagctgaggtccttgtataacacagtggctaccctctactgcgtacaccagaggatcgagattaaggataccaaggaggccttggacaaaattgaggaggagcaaaacaagagcaagaagaaggcccagcaggcagctgctgacactgggcatagcaaccaggtatcacagaactatcctattgtccaaaacattcagggccagatggttcatcaggccatcagcccccggacgctcaatgcctgggtgaaggttgtcgaagagaaggccttttctcctgaggttatccccatgttctccgctttgagtgagggggccactcctcaggacctcaatacaatgcttaataccgtgggcggccatcaggccgccatgcaaatgttgaaggagactatcaacgaggaggcagccgagtgggacagagtgcatcccgtccacgctggcccaatcgcgcccggacagatgcgggagcctcgcggctctgacattgccggcaccacctctacactgcaagagcaaatcggatggatgaccaacaatcctcccatcccagttggagaaatctataaacggtggatcatcctgggcctgaacaagatcgtgcgcatgtactctccgacatccatccttgacattagacagggacccaaagagccttttagggattacgtcgaccggttttataagaccctgcgagcagagcaggcctctcaggaggtcaaaaactggatgacggagacactcctggtacagaacgctaaccccgactgcaaaacaatcttgaaggcactaggcccggctgccaccctggaagagatgatgaccgcctgtcagggagtaggcggacccggacacaaagccagagtgttgatgggccccatcagtcccatcgagaccgtgccggtgaagctgaaacccgggatggacggccccaaggtcaagcagtggccactcaccgaggagaagatcaaggccctggtggagatctgcaccgagatggagaaagagggcaagatcagcaagatcgggcctgagaacccatacaacacccccgtgtttgccatcaagaagaaggacagcaccaagtggcgcaagctggtggatttccgggagctgaataagcggacccaggatttctgggaggtccagctgggcatcccccatccggccggcctgaagaagaagaagagcgtgaccgtgctggacgtgggcgacgcttacttcagcgtccctctggacgaggactttagaaagtacaccgcctttaccatcccatctatcaacaacgagacccctggcatcagatatcagtacaacgtcctcccccagggctggaagggctctcccgccattttccagagctccatgaccaagatcctggagccgtttcggaagcagaaccccgatatcgtcatctaccagtacatggacgacctgtacgtgggctctgacctggaaatcgggcagcatcgcacgaagattgaggagctgaggcagcatctgctgagatggggcctgaccactccggacaagaagcatcagaaggagccgccattcctgaagatgggctacgagctccatcccgacaagtggaccgtgcagcctatcgtcctccccgagaaggacagctggaccgtgaacgacatccagaagctggtgggcaagctcaactgggctagccagatctatcccgggatcaaggtgcgccagctctgcaagctgctgcgcggcaccaaggccctgaccgaggtgattcccctcacggaggaagccgagctcgagctggctgagaaccgggagatcctgaaggagcccgtgcacggcgtgtactatgacccctccaaggacctgatcgccgaaatccagaagcagggccaggggcagtggacataccagatttaccaggagcctttcaagaacctcaagaccggcaagtacgcccgcatgaggggcgcccacaccaacgatgtcaagcagctgaccgaggccgtccagaagatcacgaccgagtccatcgtgatctgggggaagacacccaagttcaagctgcctatccagaaggagacctgggagacgtggtggaccgaatattggcaggccacctggattcccgagtgggagttcgtgaatacacctcctctggtgaagctgtggtaccagctcgagaaggagcccatcgtgggcgcggagacattctacgtggacggcgcggccaaccgcgaaacaaagctcgggaaggccgggtacgtcaccaaccggggccgccagaaggtcgtcaccctgaccgacaccaccaaccagaagacggagctgcaggccatctatctcgctctccaggactccggcctggaggtgaacatcgtgacggacagccagtacgcgctgggcattattcaggcccagccggaccagtccgagagcgaactggtgaaccagattatcgagcagctgatcaagaaagagaaggtctacctcgcctgggtcccggcccataagggcattggcggcaacgagcaggtcgacaagctggtgagtgcggggattagaaaggtgctgatggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattggcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctga

SEQ ID NO: 2 - protein, 1067 aa, Artificial Sequence
  Tgrn plasmid insert
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLMGPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLKMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLMVGFPVTPQVPLRPMTYKAAVDLSHFLKEKGGLEGLIHSQRRQDILDLWIYHTQGYFPDWQNYTPGPGVRYPLTFGWCYKLVPVEPDKVEEANKGENTSLLHPVSLHGMDDPEREVLEWRFDSRLAFHHVARELHPEYFKNC

SEQ ID NO: 3 - DNA, 50 bp, Artificial Sequence
  Synthetic linker
aatttaaatacgtagcgcactagtcgcgctaagcgcggatatcatttaaa

SEQ ID NO: 4 - DNA, 49 bp, Artificial Sequence
  Synthetic linker
tatttaaatgatatccgcgcttaagcgcgactagtgcgctacgtattta

SEQ ID NO: 5 - DNA, 68 bp, Artificial Sequence
  Primer
gaattcgcggccgcgatgggccccatcagtcccatcgagaccgtgccggtgaagctgaaacccgggat

SEQ ID NO: 6 - DNA, 44 bp, Artificial Sequence
  Primer
ggtgtgactggaaaacccaccatcagcacctttctaatccccgc

SEQ ID NO: 7 - DNA, 23 bp, Artificial Sequence
  Primer
atggtgggttttccagtcacacc

SEQ ID NO: 8 - DNA, 29 bp, Artificial Sequence
  Primer
gatgaaatgctaggcggctgtcaaacctc

SEQ ID NO: 9 - DNA, 29 bp, Artificial Sequence
  Primer
gaggtttgacagccgcctagcatttcatc

SEQ ID NO: 10 - DNA, 31 bp, Artificial Sequence
  Primer
cgcggatcctcagcagttcttgaagtactcc

SEQ ID NO: 11 - DNA, 68 bp, Artificial Sequence; identical to SEQ ID NO: 5
  Primer

SEQ ID NO: 12 - DNA, 39 bp, Artificial Sequence
  Primer
ggagctcgtagcccatcttcaggaatggcggctccttct

SEQ ID NO: 13 - DNA, 68 bp, Artificial Sequence
  Primer
gaattcggatccttacagcacctttctaatccccgcactcaccagcttgtcgacctgctcgttgccgc

SEQ ID NO: 14 - DNA, 26 bp, Artificial Sequence
  Primer
cctgaagatgggctacgagctccatg